(19) United States
(12) Patent Application Publication
(10) Pub. No.: US 2018/0009785 A1
(43) Pub. Date: Jan. 11, 2018

(54) SUBSTITUTED HYDANTOIN AND THIOHYDANTOIN DERIVATIVES AS ANDROGEN RECEPTOR ANTAGONISTS

(71) Applicant: JANSSEN PHARMACEUTICA NV, BEERSE (BE)

(72) Inventor: Gilles Bignan, Bridgewater, NJ (US)

(21) Appl. No.: 15/643,979

(22) Filed: Jul. 7, 2017

Related U.S. Application Data
(60) Provisional application No. 62/359,995, filed on Jul. 8, 2016.

(51) Int. Cl. C07D 401/14; A61K 31/573; A61K 31/454; A61K 31/4545; C07D 401/12; A61K 31/58

(52) U.S. Cl. CPC C07D 401/14; C07D 401/12; A61K 31/58; A61K 31/454; A61K 31/4545; A61K 31/573

(57) ABSTRACT

Disclosed are compounds, compositions and methods for treating of disorders that are affected by the antagonism of one or more androgen receptor types. Such compounds are represented by Formula (I) as follows:

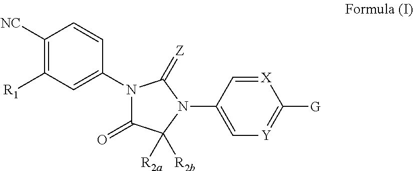

wherein R₁, R₂ₐ, R_{2b}, Z, X, Y, and G are defined herein.

### CROSS-REFERENCE TO RELATED APPLICATIONS

Not applicable.

### FIELD OF THE INVENTION

The present invention relates to novel compounds that are androgen receptor antagonists and are useful for the treatment of disorders that are affected by the modulation of the androgen receptor (AR). The invention also relates to pharmaceutical compositions comprising one or more of such compounds, to processes to prepare such compounds and compositions, and to the use of such compounds or pharmaceutical compositions for the treatment of prostate cancer and diseases, syndromes, disorders, or conditions associated with an AR mutant associated with castration-resistant prostate cancer.

### BACKGROUND OF THE INVENTION

Prostate cancer is the most common non-cutaneous malignancy in men and the second leading cause of death in men from cancer in the western world. As a male sexual organ, development of the prostate is highly regulated by androgens, the AR and by the products of androgen dependent genes. During all stages of prostate cancer progression, the disease remains dependent upon androgens. Anti-androgens, including AR antagonists, are used therapeutically to reverse the dependence of the tumor upon the actions of androgen (Scher H, Sawyers C. Biology of progressive, castration-resistant prostate cancer: directed therapies targeting the androgen-receptor signaling axis. J Clin Oncol 2005; 23:8253-8261; Tran C, Ouk S, Clegg N, Chen Y, Watson P, Arora V, et al. Development of a second-generation antiandrogen for treatment of advanced prostate cancer. Science 2009; 324:787-790; Scher H, Fizazi K, Saad F, Taplin M, Sternberg C, Miller K, et al. Increased survival with enzalutamide in prostate cancer after chemotherapy. N Engl J Med 2012; 367:1187-1197). Unfortunately, the efficacy of even second-generation, highly potent AR antagonists, such as MDV-3100 (enzalutamide, Xtandi®), is short-lived in many patients.

AR antagonists have transformed patient care by targeting a key nodal point in tumor cell signaling. However, as with other molecularly targeted cancer therapies across different oncology indications, the emergence of acquired resistance via mutation of the therapeutic target is not uncommon. This is best exemplified by imatinib-treated patients with chronic myeloid leukemia in whom ABL kinase mutations render leukemia cells resistant to imatinib. Multiple next-generation ABL inhibitors have since been developed to circumvent the mutation and with activity in this setting (Gorre M, Mohammed M, Ellwood K, Hsu N, Paquette R, Rao P, Sawyers C. Clinical resistance to STI-571 cancer therapy caused by BCRABL gene mutation or amplification. Science 2001, 293:876-80; O'Hare T, Deininger M W, Eide C A, Clackson T, Druker B J. Targeting the BCR-ABL signaling pathway in therapy-resistant Philadelphia chromosome-positive leukemia. Clin Cancer Res 2011, 17: 212-21).

Importantly, the activity of second- and third-generation AR inhibitors indicates that the disease remains “addicted” to a deregulated driver. This has led to the paradigm of sequential therapy targeting the same driver oncogene in distinct resistant states and is applicable herein to targeting of AR and the lineage dependence of AR signaling.

AR mutations that result in receptor promiscuity and the ability of these anti-androgens to exhibit agonist activity might at least partially account for this phenomenon. For example, hydroxyflutamide and bicalutamide act as AR agonists in T877A and W741L/W741C AR mutants, respectively.

In the setting of prostate cancer cells that were rendered castration resistant via overexpression of AR, it has been demonstrated that certain anti-androgen compounds, such as bicalutamide, have a mixed antagonist/agonist profile (Tran C, Ouk S, Clegg N, Chen Y, Watson P, Arora V, et al. Development of a second-generation antiandrogen for treatment of advanced prostate cancer. Science 2009, 324:787-790). This agonist activity helps to explain a clinical observation, called the anti-androgen withdrawal syndrome, whereby about 30% of men who progress on AR antagonists experience a decrease in serum PSA when therapy is discontinued (Scher, H. I. and Kelly, W. K., J Urol 1993 March, 149(3): 607-9). Prostate specific antigen decline after antiandrogen withdrawal: the flutamide withdrawal syndrome.

Accumulating evidence indicates that castration-resistant prostate cancer (CRPC) remains dependent upon AR signaling through reactivation of AR signaling (Yuan X, Balk S. Mechanisms mediating androgen receptor reactivation after castration. Urol Oncol 2009; 27: 36-41; Linja M, Savinainen K, Saramaki O, Tammela T, Vessella R, Visakorpi T. Amplification and overexpression of androgen receptor gene in hormone-refractory prostate cancer. Cancer Res 2001, 61:3550-5; Chen C, Welsbie D, Tran C, Baek S, Chen R, Vessella R, Rosenfeld M, Sawyers C. Molecular determinants of resistance to antiandrogen therapy. Nat Med 2004, 10(1): 33-9). Point mutation in the ligand-binding domain (LBD) of AR accounts for 10-20% of resistance and is characterized by receptor activation, rather than inhibition, by anti-androgen drugs (Beltran H, Yelensky R, Frampton G, Park K, Downing S, MacDonald T, et al. Targeted next-generation sequencing of advanced prostate cancer identifies potential therapeutic targets and disease heterogeneity. Eur Urol 2013, 63(5): 920-6; Bergerat J, Céraline J. Pleiotropic functional properties of androgen receptor mutants in prostate cancer. Hum Mutat 2009, 30(2): 145-57). Many of these mutations broaden ligand specificity, and some confer resistance by converting the AR antagonist into an agonist of the mutant receptor (Veldscholte J, Ris-Stalpers C, Kuiper G G, Jenster G, Berrevoets C, Claassen E, van Rooij H C, Trapman J, Brinkmann A O, Mulder E. A mutation in the ligand binding domain of the androgen receptor of human LNCaP cells affects steroid binding characteristics and response to anti-androgens. Biochem Biophys Res Commun. 1990, 173: 534-40; Haapala K, Hyytinen E, Roiha M, Laurila M, Rantala I, Helin H, Koivisto P. Androgen receptor alterations in prostate cancer relapsed during a combined androgen blockade by orchiectomy and bicalutamide. Lab Invest 2001, 81(12):1647-1651; Hara T, Miyazaki J, Araki H, Yamaoka M, Kanzaki N, Kusaka M, Miyamoto M. Novel mutations of androgen receptor: a possible mechanism of bicalutamide withdrawal syndrome. Cancer Res 2003, 63(1):149-153).

One mutation, phenylalanine to leucine at position 876 (F876L) of AR, was recently shown to arise in response to MDV-3100 and ARN-509 in preclinical models and in patients undergoing therapy with ARN-509 (Clegg N, Wongvipat J, Joseph J, Tran C, Ouk S, Dilhas A, et al. ARN-509: a novel antiandrogen for prostate cancer treatment. Cancer Res 2012, 72(6): 1494-503; Balbas M, Evans M, Hosfield D, Wongvipat J, Arora V, Watson P, et al. Overcoming mutation-based resistance to antiandrogens with rational drug design. Elife 2013. 2: e00499; Korpal M, Korn J, Gao X, Rakiec D, Ruddy D, Doshi S, et al. An F876L mutation in androgen receptor confers genetic and phenotypic resistance to MDV3100 (enzalutamide). Cancer Discov 2013, 39:1030-1043; Joseph J D, Lu N, Qian J, Sensintaffar J, Shao G, Brigham D, Moon M, Maneval E C, Chen I, Darimont B, Hager J H. A clinically relevant androgen receptor mutation confers resistance to second-generation antiandrogens enzalutamide and ARN-509. Cancer Discov 2013, 3:1020-1029).

AR F876L confers resistance to MDV-3100 and ARN-509. Comprehensive biological studies have demonstrated that prostate cancer cells harboring this mutation continued to grow when treated with either compound. In vitro reporter assays confirmed resistance and demonstrate agonist conversion of both compounds and in tumors engineered to express AR F876L, neither compound controlled tumor growth. Furthermore, the AR F876L mutant is detected in ARN-509-treated patients with progressive CRPC. The mutation was detected in the plasma DNA of patients undergoing longitudinal analysis in 3 of 29 patients eligible for assessment. All 3 of the patients were amongst the 18 patients with an increase in prostate specific antigen (PSA) whilst on drug, indicative of disease progression (Joseph 2013).

Structural modeling of wild-type (WT) and F876L mutated AR bound with MDV-3100, indicated that helices 11 and 12 were differentially displaced. Within the LBD of AR in the F876L mutant, helix 12 is not displaced by MDV-3100 as it is in WT AR, and this allows MDV 3100 to function as an agonist. The compounds described herein are designed to act as antagonists (third-generation), where second-generation compounds are not active.

Thus, androgen receptor antagonists of the present invention may provide therapeutic benefit for the treatment of prostate cancer and other diseases, syndromes, disorders, or conditions associated with an AR mutant associated with castration-resistant prostate cancer.

### SUMMARY OF THE INVENTION

The present invention is directed to compounds of Formula (I)

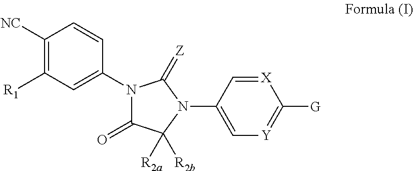

wherein

Z is S or O;

R₁ is chloro, methyl, methoxy, difluoromethyl, or trifluoromethyl;

R₂ₐ and R_{2b} are independently C₁₋₆alkyl; or, R₂ₐ and R_{2b} are taken together with the carbon atom to which they are attached to form an unsubstituted or substituted C₃-C₁₀cycloalkyl or an unsubstituted or substituted C₃-C₁₀heterocyclyl selected from the group consisting of pyrrolidinyl and piperidinyl, wherein said substituted C₃₋₁₀cycloalkyl or substituted C₃-C₁₀heterocyclyl are optionally independently substituted with a C₁₋₃alkyl or cyclopropyl substituent;

X is C or N;

Y is C or N;

G is selected from the group consisting of g1 and g2

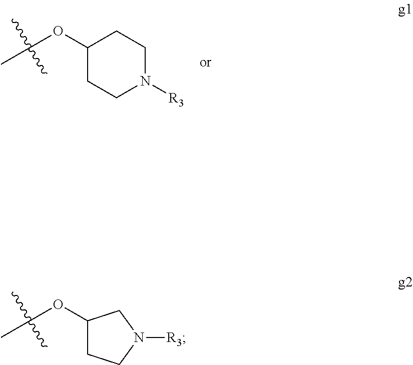

wherein R₃ is selected from the group consisting of hydrogen; C₁₋₆alkyl optionally independently substituted with a substituent selected from hydroxy, methoxy, cyano, or fluoro; C₃₋₆cycloalkyl optionally independently substituted with a substituent selected from hydroxy or fluoro; and —C(O)OR₄, wherein R₄ is C₁₋₆alkyl or —CH₂(C₆₋₁₀aryl) wherein C₆₋₁₀aryl is optionally substituted with a methoxy substituent;

such that a substituent on C₁₋₆alkyl or C₃₋₆cycloalkyl is attached at a carbon atom other than the carbon atom directly attached to the G-nitrogen atom;

wherein any nitrogen-containing heterocyclic substituent of G is optionally substituted with an oxido substituent to form an N-oxide; or an enantiomer, diastereomer, or pharmaceutically acceptable salt form thereof.

The present invention also provides a pharmaceutical composition comprising, consisting of and/or consisting essentially of a pharmaceutically acceptable carrier, a pharmaceutically acceptable excipient, and/or a pharmaceutically acceptable diluent and a compound of Formula (I), or a pharmaceutically acceptable salt form thereof.

Also provided are processes for making a pharmaceutical composition comprising, consisting of, and/or consisting essentially of admixing a compound of Formula (I), and a pharmaceutically acceptable carrier, a pharmaceutically acceptable excipient, and/or a pharmaceutically acceptable diluent.

The present invention further provides methods for treating or ameliorating a disease, syndrome, condition, or disorder in a subject, including a mammal and/or human in which the disease, syndrome, or condition is affected by the antagonism of the androgen receptor, such as prostate cancer and further diseases, syndromes, disorders, or conditions associated with an AR mutant associated with castration-resistant prostate cancer, using a compound of Formula (I).

The present invention also is directed to the use of any of the compounds described herein in the preparation of a medicament wherein the medicament is prepared for treating a disease, syndrome, condition, or disorder that is affected by the antagonism of one or more androgen receptor types, such as prostate cancer, castration-resistant prostate cancer, and metastatic castration-resistant prostate cancer.

The present invention is also directed to the preparation of substituted hydantoin and thiohydantoin derivatives that act as antagonists of one or more androgen receptors.

Exemplifying the invention are methods of treating a disease, syndrome, condition, or disorder mediated by one or more androgen receptors, selected from the group consisting of prostate cancer, castration-resistant prostate cancer, and metastatic castration-resistant prostate cancer, comprising, consisting of, and/or consisting essentially of, administering to a subject in need thereof a therapeutically effective amount of any of the compounds or pharmaceutical compositions described in the present invention.

In another embodiment, the present invention is directed to a compound of Formula (I) for use in the treatment of a disease, syndrome, condition, or disorder affected by the antagonism of one or more androgen receptor types, selected from the group consisting of prostate cancer, castration-resistant prostate cancer, and metastatic castration-resistant prostate cancer.

In another embodiment, the present invention is directed to a composition comprising a compound of Formula (I) for the treatment of a disease, syndrome, condition, or disorder affected by the antagonism of one or more androgen receptors, selected from the group consisting of prostate cancer, castration-resistant prostate cancer, and metastatic castration-resistant prostate cancer.

Another embodiment of the present invention is directed to a pharmaceutical composition comprising a compound of Formula (I).

### DETAILED DESCRIPTION OF THE INVENTION

With reference to substituents, the term “independently” refers to the situation where when more than one substituent is possible, the substituents may be the same or different from each other.

The term “alkyl” whether used alone or as part of a substituent group, refers to straight and branched carbon chains having 1 to 8 carbon atoms. Therefore, designated numbers of carbon atoms (e.g., C₁₋₈) refer independently to the number of carbon atoms in an alkyl moiety or to the alkyl portion of a larger alkyl-containing substituent. In substituent groups with multiple alkyl groups such as, (C₁₋₆alkyl)₂amino-, the C₁₋₆alkyl groups of the dialkylamino may be the same or different.

The term “alkoxy” refers to an —O-alkyl group, wherein the term “alkyl” is as defined above.

The terms “alkenyl” and “alkynyl” refer to straight and branched carbon chains having 2 to 8 carbon atoms, wherein an alkenyl chain contains at least one double bond and an alkynyl chain contains at least one triple bond.

The term “cycloalkyl” refers to saturated or partially saturated, monocyclic or polycyclic hydrocarbon rings of 3 to 14 carbon atoms. Examples of such rings include cyclopropyl, cyclobutyl, cyclopentyl, cyclohexyl, cycloheptyl, and adamantyl.

The term “heterocyclyl” refers to a nonaromatic monocyclic or bicyclic ring system having 3 to 10 ring members that include at least 1 carbon atom and from 1 to 4 heteroatoms independently selected from N, O, and S. Included within the term heterocyclyl is a nonaromatic cyclic ring of 5 to 7 members in which 1 to 2 members are N, or a nonaromatic cyclic ring of 5 to 7 members in which 0, 1 or 2 members are N and up to 2 members are O or S and at least one member must be either N, O, or S; wherein, optionally, the ring contains 0 to 1 unsaturated bonds, and, optionally, when the ring is of 6 or 7 members, it contains up to 2 unsaturated bonds. The carbon atom ring members that form a heterocycle ring may be fully saturated or partially saturated. The term “heterocyclyl” also includes two 5 membered monocyclic heterocycloalkyl groups bridged to form a bicyclic ring. Such groups are not considered to be fully aromatic and are not referred to as heteroaryl groups. When a heterocycle is bicyclic, both rings of the heterocycle are non-aromatic and at least one of the rings contains a heteroatom ring member. Examples of heterocycle groups include, and are not limited to, pyrrolinyl (including 2H-pyrrole, 2-pyrrolinyl or 3-pyrrolinyl), pyrrolidinyl, imidazolinyl, imidazolidinyl, pyrazolinyl, pyrazolidinyl, piperidinyl, morpholinyl, thiomorpholinyl, and piperazinyl. Unless otherwise noted, the heterocycle is attached to its pendant group at any heteroatom or carbon atom that results in a stable structure.

The term “aryl” refers to an unsaturated, aromatic monocyclic or bicyclic ring of 6 to 10 carbon members. Examples of aryl rings include phenyl and naphthalenyl. The term “heteroaryl” refers to an aromatic monocyclic or bicyclic aromatic ring system having 5 to 10 ring members and which contains carbon atoms and from 1 to 4 heteroatoms independently selected from the group consisting of N, O, and S. Included within the term heteroaryl are aromatic rings of 5 or 6 members wherein the ring consists of carbon atoms and has at least one heteroatom member. Suitable heteroatoms include nitrogen, oxygen, and sulfur. In the case of 5 membered rings, the heteroaryl ring preferably contains one member of nitrogen, oxygen or sulfur and, in addition, up to 3 additional nitrogens. In the case of 6 membered rings, the heteroaryl ring preferably contains from 1 to 3 nitrogen atoms. For the case wherein the 6 membered ring has 3 nitrogens, at most 2 nitrogen atoms are adjacent. Examples of heteroaryl groups include furyl, thienyl, pyrrolyl, oxazolyl, thiazolyl, imidazolyl, pyrazolyl, isoxazolyl, isothiazolyl, oxadiazolyl, triazolyl, thiadiazolyl, pyridinyl, pyridazinyl, pyrimidinyl, pyrazinyl, indolyl, isoindolyl, benzofuryl, benzothienyl, indazolyl, benzimidazolyl, benzothiazolyl, benzoxazolyl, benzisoxazolyl, benzothiadiazolyl, benzotriazolyl, quinolinyl, isoquinolinyl and quinazolinyl. Unless otherwise noted, the heteroaryl is attached to its pendant group at any heteroatom or carbon atom that results in a stable structure.

The term “halogen” or “halo” refers to fluorine, chlorine, bromine and iodine atoms.

The term “carboxy” refers to the group —C(═O)OH.

The term “formyl” refers to the group —C(═O)H.

The term “oxo” or “oxido” refers to the group (═O).

Whenever the term “alkyl” or “aryl” or either of their prefix roots appear in a name of a substituent (e.g., arylalkyl, alkylamino) the name is to be interpreted as including those limitations given above for “alkyl” and “aryl.” Designated numbers of carbon atoms (e.g., C₁-C₆) refer independently to the number of carbon atoms in an alkyl moiety, an aryl moiety, or in the alkyl portion of a larger substituent in which alkyl appears as its prefix root. For alkyl and alkoxy substituents, the designated number of carbon atoms includes all of the independent members included within a given range specified. For example C₁₋₆ alkyl would include methyl, ethyl, propyl, butyl, pentyl and hexyl individually as well as sub-combinations thereof (e.g., C₁₋₂, C₁₋₃, C₁₋₄, C₁₋₅, C₂₋₆, C₃₋₆, C₄₋₆, C₅₋₆, C₂₋₅, etc.).

In general, under standard nomenclature rules used throughout this disclosure, the terminal portion of the designated side chain is described first followed by the adjacent functionality toward the point of attachment. Thus, for example, a “C₁-C₆ alkylcarbonyl” substituent refers to a group of the formula:

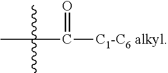

The label “R” at a stereocenter designates that the stereocenter is purely of the R-configuration as defined in the art; likewise, the label “S” means that the stereocenter is purely of the S-configuration. As used herein, the labels “*R” or “*S” at a stereocenter are used to designate that the stereocenter is of pure but unknown absolute configuration. As used herein, the label “RS” refers to a stereocenter that exists as a mixture of the R- and S-configurations.

A compound containing one stereocenter drawn without a stereo bond designation is a mixture of two enantiomers. A compound containing two stereocenters both drawn without stereo bond designations is a mixture of four diastereomers. A compound with two stereocenters both labeled “RS” and drawn with stereo bond designations is a mixture of two enantiomers with relative stereochemistry as drawn. A compound with two stereocenters both labeled “*RS” and drawn with stereo bond designations is a mixture of two enantiomers with a single, but unknown, relative stereochemistry.

Unlabeled stereocenters drawn without stereo bond designations are mixtures of the R- and S-configurations. For unlabeled stereocenters drawn with stereo bond designations, the relative and absolute stereochemistry is as depicted.

Unless otherwise noted, it is intended that the definition of any substituent or variable at a particular location in a molecule be independent of its definitions elsewhere in that molecule. It is understood that substituents and substitution patterns on the compounds of the present invention can be selected by one of ordinary skill in the art to provide compounds that are chemically stable and that can be readily synthesized by techniques known in the art as well as those methods set forth herein.

The term “subject” refers to an animal, preferably a mammal, most preferably a human, who has been the object of treatment, observation or experiment.

The term “therapeutically effective amount” refers to an amount of an active compound or pharmaceutical agent, including a compound of the present invention, which elicits the biological or medicinal response in a tissue system, animal or human that is being sought by a researcher, veterinarian, medical doctor or other clinician, which includes alleviation or partial alleviation of the symptoms of the disease, syndrome, condition, or disorder being treated.

The term “composition” refers to a product that includes the specified ingredients in therapeutically effective amounts, as well as any product that results, directly, or indirectly, from combinations of the specified ingredients in the specified amounts.

The term “androgen receptor” as used herein is intended to include the wild-type androgen receptor as well as AR mutants associated with castration-resistant prostate cancer.

The term “AR-mediated” refers to any disease, syndrome, condition, or disorder that might occur in the absence of androgen receptors but can occur in the presence of androgen receptors. Suitable examples of include, but are not limited to, prostate cancer, castration-resistant prostate cancer, and metastatic castration-resistant prostate cancer.

The term “Androgen-dependent disorder” refers to any disorder that can benefit from a decrease in androgen stimulation and includes pathological conditions that depend on androgen stimulation. An “androgen-dependent disorder” can result from an excessive accumulation of testosterone or other androgenic hormone, increased sensitivity of androgen receptors to androgen, or an increase in androgen-stimulated transcription.

Examples of “androgen-dependent disorders” include prostate cancer and disorders such as, for example, acne, seborrhea, hirsutism, alopecia, and hidradenitis suppurativa.

As used herein, the term “anti-androgen” refers to a group of hormone receptor antagonist compounds that are capable of preventing or inhibiting the biologic effects of androgens on normally responsive tissues in the body. In some embodiments, an anti-androgen is a small molecule. In some embodiments, an anti-androgen is an AR antagonist. In some embodiments, an anti-androgen is an AR full antagonist. In some embodiments, an anti-androgen is a first-generation anti-androgen. In some embodiments, an anti-androgen is a second-generation anti-androgen. In some embodiments, an anti-androgen is a third-generation anti-androgen.

As used herein, the term “AR antagonist” or “AR inhibitor” are used interchangeably and refer to an agent that inhibits or reduces at least one activity of an AR polypeptide. Exemplary AR activities include, but are not limited to, co-activator binding, DNA binding, ligand binding, or nuclear translocation.

As used herein, a “full antagonist” refers to an antagonist which, at an effective concentration, essentially completely inhibits an activity of an AR polypeptide. As used herein, a “partial antagonist” refers an antagonist that is capable of partially inhibiting an activity of an AR polypeptide, but that, even at a highest concentration is not a full antagonist. By ‘essentially completely’ is meant at least about 80%, at least about 90%, at least about 95%, at least about 96%, at least about 97%, at least about 98% at least about 99%, or greater inhibition of the activity of an AR polypeptide.

As used herein, the term “first-generation anti-androgen” refers to an agent that exhibits antagonist activity against a wild-type AR polypeptide. However, first-generation anti-androgens differ from second-generation anti-androgens in that first-generation anti-androgens can potentially act as agonists in castration resistant prostate cancers (CRPC).

Exemplary first-generation anti-androgens include, but are not limited to, flutamide, nilutamide and bicalutamide.

As used herein, the term “second-generation anti-androgen” refers to an agent that exhibits full antagonist activity against a wild-type AR polypeptide. Second-generation anti-androgens differ from first-generation anti-androgens in that second-generation anti-androgens act as full antagonists in cells expressing elevated levels of AR, such as for example, in castration resistant prostate cancers (CRPC). Exemplary second-generation anti-androgens include 4-[7-(6-cyano-5-trifluoromethylpyridin-3-yl)-8-oxo-6-thioxo-5,7-diazaspiro[3.4]oct-5-yl]-2-fluoro-N methylbenzamide (also known as ARN-509; CAS No. 956104-40-8); 4-(3-(4-cyano-3-(trifluoromethyl)phenyl)-5,5-dimethyl-4-oxo-2-thioxoimidazolidin-1-yl)-2-fluoro-N-methylbenzamide (also known as MDV3100 or enzalutamide; CAS No: 915087-33-1) and RD162 (CAS No. 915087-27-3). In some embodiments, a second-generation anti-androgen binds to an AR polypeptide at or near the ligand binding site of the AR polypeptide.

As used herein, the term “third-generation anti-androgen” refers to an agent that exhibits full antagonist activity against a wild-type AR polypeptide and against mutant forms of the AR polypeptide, with mutations arising in the ligand binding domain (LBD) of the AR polypeptide as set forth below. Third-generation anti-androgens retain the differentiation from first-generation anti-androgens in that third-generation anti-androgens act as full antagonists in cells expressing elevated levels of AR, such as for example, in castration resistant prostate cancers (CRPC).

As used herein, the term “mutant” refers to an altered (as compared with a reference) nucleic acid or polypeptide, or to a cell or organism containing or expressing such altered nucleic acid or polypeptide.

As used herein, unless otherwise noted, the term “affect” or “affected” (when referring to a disease, syndrome, condition or disorder that is affected by antagonism of AR) includes a reduction in the frequency and/or severity of one or more symptoms or manifestations of said disease, syndrome, condition or disorder; and/or include the prevention of the development of one or more symptoms or manifestations of said disease, syndrome, condition or disorder or the development of the disease, condition, syndrome or disorder.

The compounds of the instant invention are useful in methods for treating or ameliorating a disease, a syndrome, a condition or a disorder that is affected by the antagonism of one or more AR receptors. Such methods comprise, consist of and/or consist essentially of administering to a subject, including an animal, a mammal, and a human in need of such treatment, amelioration and/or prevention, a therapeutically effective amount of a compound of Formula (I), or an enantiomer, diastereomer, solvate or pharmaceutically acceptable salt thereof.

One embodiment of the present invention is directed to a method of treating an androgen receptor dependent or androgen receptor mediated disease or condition in a subject in need thereof, including an animal, a mammal, and a human in need of such treatment, comprising administering to the subject a therapeutically effective amount of a compound of Formula (I).

In another embodiment, the androgen receptor dependent or androgen receptor mediated disease or condition is selected from benign prostate hyperplasia, hirsutism, acne, adenomas and neoplasies of the prostate, benign or malignant tumor cells containing the androgen receptor, hyperpilosity, seborrhea, endometriosis, polycystic ovary syndrome, androgenic alopecia, hypogonadism, osteroporosis, suppression of spermatogenesis, libido, cachexia, anorexia, androgen supplementation for age related decreased testosterone levels, prostate cancer, breast cancer, endometrial cancer, uterine cancer, hot flashes, and Kennedy's disease muscle atrophy and weakness, skin atrophy, bone loss, anemia, arteriosclerosis, cardiovasculasr disease, loss of energy, loss of well-being, type 2 diabetes, or abdominal fat accumulation.

In particular, the compounds of Formula (I), or an enantiomer, diastereomer, solvate or pharmaceutically acceptable salt form thereof are useful for treating or ameliorating diseases, syndromes, conditions, or disorders such as prostate cancer, castration-resistant prostate cancer, and metastatic castration-resistant prostate cancer.

More particularly, the compounds of Formula (I), or an enantiomer, diastereomer, solvate or pharmaceutically acceptable salt form thereof, are useful for treating or ameliorating prostate cancer, castration-resistant prostate cancer, and metastatic castration-resistant prostate cancer, comprising administering to a subject in need thereof a therapeutically effective amount of a compound of Formula (I), or an enantiomer, diastereomer, solvate or pharmaceutically acceptable salt form thereof as herein defined.

Embodiments of the present invention include a compound of Formula (I)

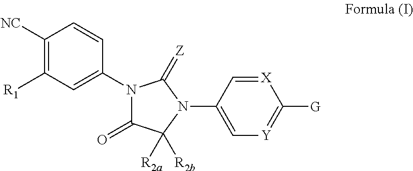

wherein
AA) Z is S;
BB) R₁ is chloro, methyl, methoxy, or trifluoromethyl;
CC) R₁ is chloro, methyl, or trifluoromethyl;
DD) R₁ is chloro or trifluoromethyl;
EE) R₂ₐ and R_{2b} are independently methyl; or, R₂ₐ and R_{2b} are taken together with the carbon atom to which they are attached to form an unsubstituted cyclobutyl ring;
FF) X is C;
GG) Y is N;
HH) G is selected from the group consisting of g1 and g2

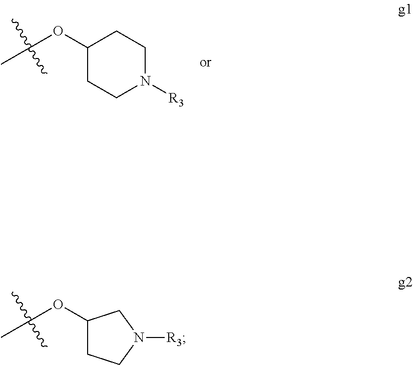

wherein R₃ is selected from the group consisting of hydrogen; C₁₋₃alkyl optionally independently substituted with a substituent selected from hydroxy, methoxy, or fluoro; C₃₋₆cycloalkyl optionally independently substituted with a substituent selected from hydroxy or fluoro; and —C(O)OR₄, wherein R₄ is C₁₋₆alkyl or —CH₂(phenyl) wherein the phenyl is optionally substituted with a methoxy substituent;
such that a substituent on C₁₋₆alkyl or C₃₋₆cycloalkyl is attached at a carbon atom other than the carbon atom directly attached to the G-nitrogen atom;
II) G is selected from the group consisting of g1 and g2

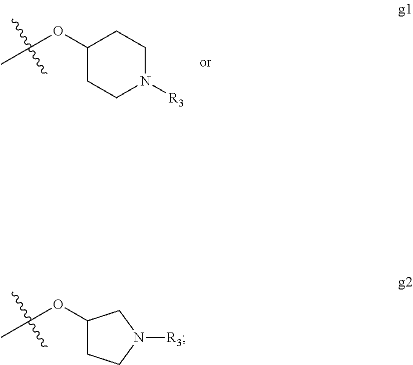

wherein R₃ is selected from the group consisting of hydrogen; C₁₋₃alkyl optionally independently substituted with a substituent selected from methoxy or fluoro; and —C(O)OR₄, wherein R₄ is C₁₋₆alkyl or —CH₂(phenyl) wherein the phenyl is optionally substituted with a methoxy substituent;
such that a substituent on C₁₋₃alkyl is attached at a carbon atom other than the carbon atom directly attached to the G-nitrogen atom;
JJ) G is selected from the group consisting of g1 and g2

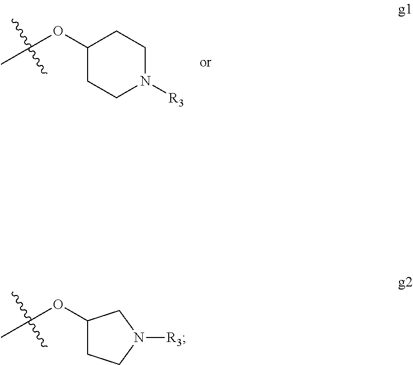

wherein R₃ is selected from the group consisting of hydrogen; methyl, and —C(O)OR₄, wherein R₄ is C₁₋₄alkyl or —CH₂(phenyl);
KK) G is g1

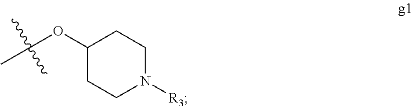

wherein R₃ is selected from the group consisting of hydrogen; methyl, and —C(O)OR₄, wherein R₄ is C₁₋₄alkyl or —CH₂(phenyl);
LL) G is g1

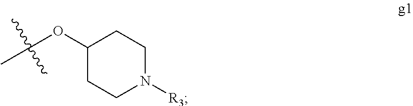

wherein R₃ is selected from the group consisting of hydrogen and methyl;

and any combination of embodiments AA) through LL) above, provided that it is understood that combinations in which different embodiments of the same substituent would be combined are excluded; wherein any nitrogen-containing heterocyclic substituent of G is optionally substituted with an oxido substituent to form an N-oxide; or an enantiomer, diastereomer, or pharmaceutically acceptable salt form thereof.

Embodiments of the present invention include a compound of Formula (I)

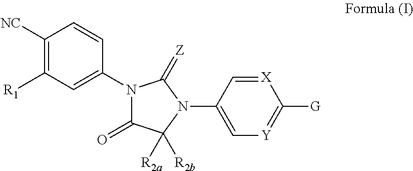

wherein

Z is S;

R₁ is chloro, methyl, methoxy, or trifluoromethyl;

R₂ₐ and R_{2b} are independently methyl; or, R₂ₐ and R_{2b} are taken together with the carbon atom to which they are attached to form an unsubstituted cyclobutyl ring;

X is C or N;

Y is C or N;

G is selected from the group consisting of g1 and g2

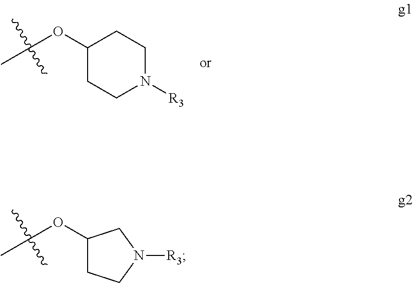

wherein R₃ is selected from the group consisting of hydrogen; C₁₋₄alkyl optionally independently substituted with a substituent selected from hydroxy, methoxy, or fluoro; C₃₋₆ cycloalkyl optionally independently substituted with a substituent selected from hydroxy or fluoro; and —C(O)OR₄, wherein R₄ is C₁₋₆alkyl or —CH₂(phenyl) and wherein the phenyl is optionally substituted with a methoxy substituent;

such that a substituent on C₁₋₄alkyl or C₃₋₆cycloalkyl is attached at a carbon atom other than the carbon atom directly attached to the G-nitrogen atom;

wherein any nitrogen-containing heterocyclic substituent of G is optionally substituted with an oxido substituent to form an N-oxide; or an enantiomer, diastereomer, or pharmaceutically acceptable salt form thereof.

Embodiments of the present invention include a compound of Formula (I)

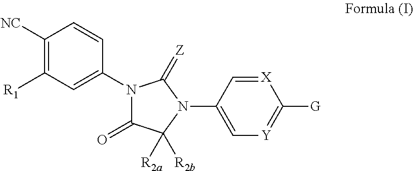

wherein

Z is S;

R₁ is chloro, methyl, or trifluoromethyl;

R₂ₐ and R_{2b} are independently methyl; or, R₂ₐ and R_{2b} are taken together with the carbon atom to which they are attached to form an unsubstituted cyclobutyl ring;

X is C or N;

Y is C or N;

G is selected from the group consisting of g1 and g2

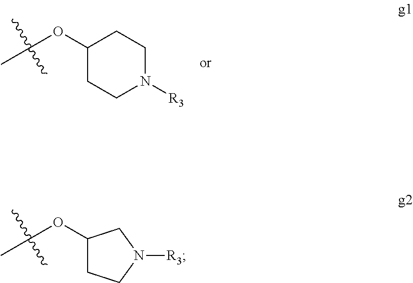

wherein R₃ is selected from the group consisting of hydrogen; C₁₋₃alkyl optionally independently substituted with a substituent selected from methoxy or fluoro; and —C(O)OR₄, wherein R₄ is C₁₋₆alkyl or —CH₂(phenyl) and wherein the phenyl is optionally substituted with a methoxy substituent;

such that a substituent on C₁₋₃alkyl is attached at a carbon atom other than the carbon atom directly attached to the G-nitrogen atom;

wherein any nitrogen-containing heterocyclic substituent of G is optionally substituted with an oxido substituent to form an N-oxide; or an enantiomer, diastereomer, or pharmaceutically acceptable salt form thereof.

Embodiments of the present invention include a compound of Formula (I)

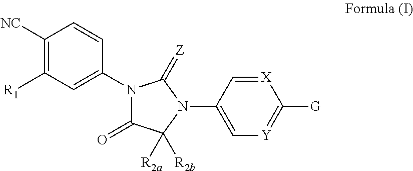

wherein

Z is S;

R₁ is chloro or trifluoromethyl;

R₂ₐ and R_{2b} are independently methyl; or, R₂ₐ and R_{2b} are taken together with the carbon atom to which they are attached to form an unsubstituted cyclobutyl ring;

X is C or N;

Y is C or N;

G is selected from the group consisting of g1 and g2

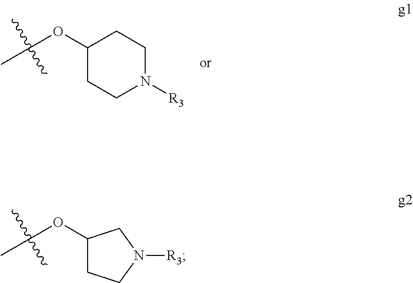

wherein R₃ is selected from the group consisting of hydrogen; methyl, and —C(O)OR₄, wherein R₄ is C₁₋₄alkyl or —CH₂(phenyl);

wherein any nitrogen-containing heterocyclic substituent of G is optionally substituted with an oxido substituent to form an N-oxide; or an enantiomer, diastereomer, or pharmaceutically acceptable salt form thereof.

Embodiments of the present invention include a compound of Formula (I)

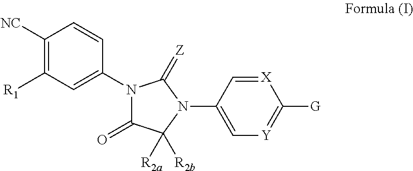

wherein

Z is S;

R₁ is chloro or trifluoromethyl;

R₂ₐ and R_{2b} are independently methyl; or, R₂ₐ and R_{2b} are taken together with the carbon atom to which they are attached to form an unsubstituted cyclobutyl ring;

X is C;

Y is N;

G is selected from the group consisting of g1 and g2

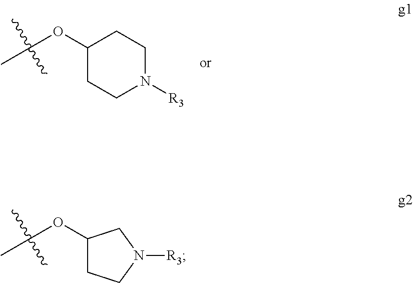

wherein R₃ is selected from the group consisting of hydrogen; methyl, and —C(O)OR₄, wherein R₄ is C₁₋₄alkyl or —CH₂(phenyl);
or an enantiomer, diastereomer, or pharmaceutically acceptable salt form thereof.

Embodiments of the present invention include a compound of Formula (I)

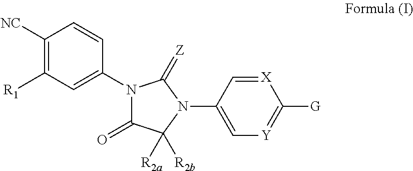

wherein

Z is S;

R₁ is chloro or trifluoromethyl;

R₂ₐ and R_{2b} are independently methyl; or, R₂ₐ and R_{2b} are taken together with the carbon atom to which they are attached to form an unsubstituted cyclobutyl ring;

X is C;

Y is N;

G is g1

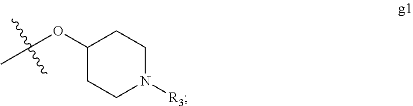

wherein R₃ is selected from the group consisting of hydrogen; methyl, and —C(O)OR₄, wherein R₄ is C₁₋₄alkyl or —CH₂(phenyl);

wherein any nitrogen-containing heterocyclic substituent of G is optionally substituted with an oxido substituent to form an N-oxide; or an enantiomer, diastereomer, or pharmaceutically acceptable salt form thereof.

Embodiments of the present invention include a compound of Formula (I)

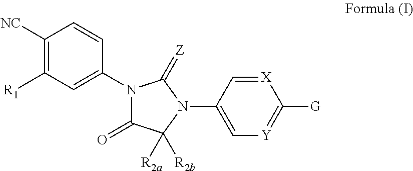

wherein

Z is S;

R₁ is chloro or trifluoromethyl;

R₂ₐ and R_{2b} are independently methyl; or, R₂ₐ and R_{2b} are taken together with the carbon atom to which they are attached to form an unsubstituted cyclobutyl ring;

X is C;

Y is N;

G is g1

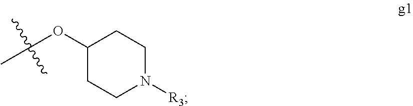

wherein R₃ is selected from the group consisting of hydrogen and methyl;

wherein any nitrogen-containing heterocyclic substituent of G is optionally substituted with an oxido substituent to form an N-oxide; or an enantiomer, diastereomer, or pharmaceutically acceptable salt form thereof.

Additional embodiments of the present invention include compounds of Formula (I) as herein defined, or an enantiomer, diastereomer, solvate, or a pharmaceutically acceptable salt form thereof, as exemplified in the listing in Table 1, below.

TABLE 1

| | Cpd | |
|---|---|---|
| Structure | No. | Cpd Name |
| 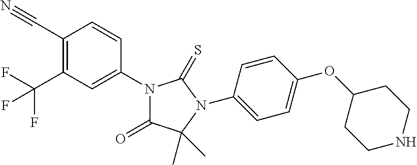 [SMILES: CC1(C)C(=O)N(c2ccc(C#N)c(C(F)(F)F)c2)C(=S)N1c1ccc(OC2CCNCC2)cc1] | 1 | 4-(4,4-Dimethyl-5-oxo-3-(4- (piperidin-4-yloxy)phenyl)-2- thioxoimidazolidin-1-yl)-2- (trifluoromethyl)benzonitrile |
| 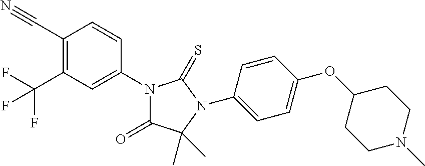 [SMILES: CN1CCC(Oc2ccc(N3C(=S)N(c4ccc(C#N)c(C(F)(F)F)c4)C(=O)C3(C)C)cc2)CC1] | 2 | 4-(4,4-Dimethyl-5-oxo-3-(4-(1- methylpiperidin-4- yloxy)phenyl)-2- thioxoimidazolidin-1-yl)-2- (trifluoromethyl)benzonitrile |
| 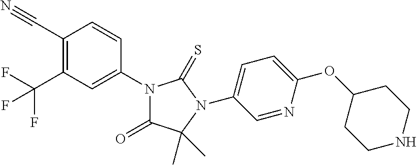 [SMILES: CC1(C)C(=O)N(c2ccc(C#N)c(C(F)(F)F)c2)C(=S)N1c1ccc(OC2CCNCC2)nc1] | 3 | 4-(4,4-Dimethyl-5-oxo-3-(6- (piperidin-4-yloxy)pyridin-3-yl)- 2-thioxoimidazolidin-1-yl)-2- (trifluoromethyl)benzonitrile |
| 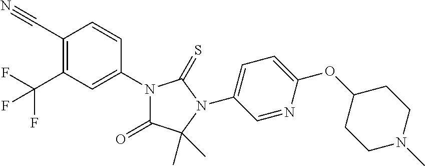 [SMILES: CN1CCC(Oc2ccc(N3C(=S)N(c4ccc(C#N)c(C(F)(F)F)c4)C(=O)C3(C)C)cn2)CC1] | 4 | 4-(4,4-Dimethyl-3-(6-((1- methylpiperidin-4- yl)oxy)pyridin-3-yl)-5-oxo-2- thioxoimidazolidin-1-yl)-2- (trifluoromethyl)benzonitrile |
| 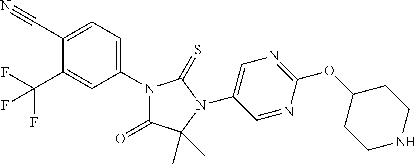 [SMILES: CC1(C)C(=O)N(c2ccc(C#N)c(C(F)(F)F)c2)C(=S)N1c1cnc(OC2CCNCC2)nc1] | 5 | 4-[4,4-Dimethyl-5-oxo-3-[2-(4- piperidinyloxy)pyrimidin-5-yl]- 2-thioxo-imidazolidin-1-yl]-2- (trifluoromethyl)benzonitrile |
| 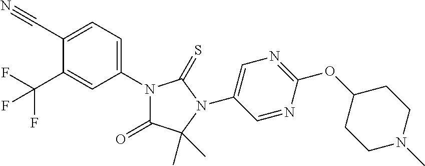 [SMILES: CN1CCC(Oc2ncc(N3C(=S)N(c4ccc(C#N)c(C(F)(F)F)c4)C(=O)C3(C)C)cn2)CC1] | 6 | 4-(4,4-Dimethyl-3-(2-((1- methylpiperidin-4- yl)oxy)pyrimidin-5-yl)-5-oxo-2- thioxoimidazolidin-1-yl)-2- (trifluoromethyl)benzonitrile |
| 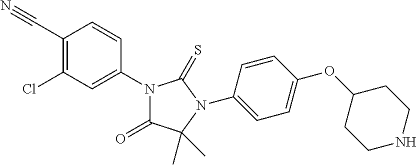 [SMILES: CC1(C)C(=O)N(c2ccc(C#N)c(Cl)c2)C(=S)N1c1ccc(OC2CCNCC2)cc1] | 7 | 2-Chloro-4-(4,4-dimethyl-5-oxo- 3-(4-(piperidin-4-yloxy)phenyl)- 2-thioxoimidazolidin-1- yl)benzonitrile |
| 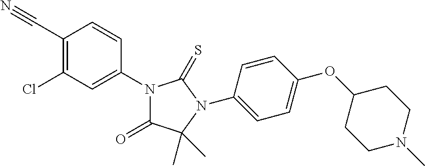 [SMILES: CN1CCC(Oc2ccc(N3C(=S)N(c4ccc(C#N)c(Cl)c4)C(=O)C3(C)C)cc2)CC1] | 8 | 2-Chloro-4-(4,4-dimethyl-3-(4- ((1-methylpiperidin-4- yl)oxy)phenyl)-5-oxo-2- thioxoimidazolidin-1- yl)benzonitrile |
| 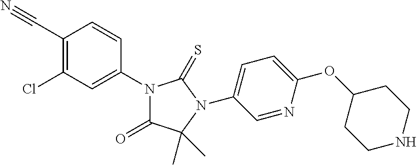 [SMILES: CC1(C)C(=O)N(c2ccc(C#N)c(Cl)c2)C(=S)N1c1ccc(OC2CCNCC2)nc1] | 9 | 2-Chloro-4-(4,4-dimethyl-5-oxo- 3-(6-(piperidin-4-yloxy)pyridin- 3-yl)-2-thioxoimidazolidin-1- yl)benzonitrile hydrochloride |
| 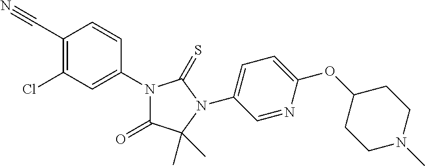 [SMILES: CN1CCC(Oc2ccc(N3C(=S)N(c4ccc(C#N)c(Cl)c4)C(=O)C3(C)C)cn2)CC1] | 10 | 2-Chloro-4-(4,4-dimethyl-3-(6- ((1-methylpiperidin-4- yl)oxy)pyridin-3-yl)-5-oxo-2- thioxoimidazolidin-1- yl)benzonitrile |
| 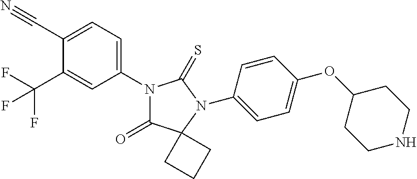 [SMILES: N#Cc1ccc(N2C(=O)C3(CCC3)N(c3ccc(OC4CCNCC4)cc3)C2=S)cc1C(F)(F)F] | 11 | 4-(8-Oxo-5-(4-(piperidin-4- yloxy)phenyl)-6-thioxo-5,7- diazaspiro[3.4]octan-7-yl)-2- (trifluoromethyl)benzonitrile |
| 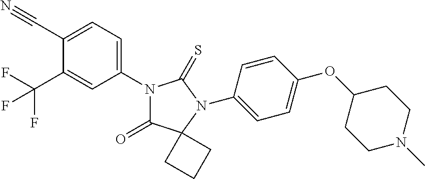 [SMILES: CN1CCC(Oc2ccc(N3C(=S)N(c4ccc(C#N)c(C(F)(F)F)c4)C(=O)C34CCC4)cc2)CC1] | 12 | 4-(5-(4-((1-Methylpiperidin-4- yl)oxy)phenyl)-8-oxo-6-thioxo- 5,7-diazaspiro[3.4]octan-7-yl)-2- (trifluoromethyl)benzonitrile |
| 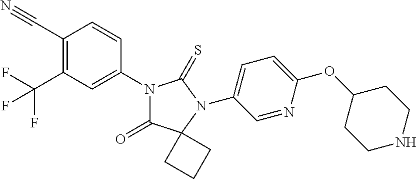 [SMILES: N#Cc1ccc(N2C(=O)C3(CCC3)N(c3ccc(OC4CCNCC4)nc3)C2=S)cc1C(F)(F)F] | 13 | 4-(8-Oxo-5-(6-(piperidin-4- yloxy)pyridin-3-yl)-6-thioxo-5,7- diazaspiro[3.4]octan-7-yl)-2- (trifluoromethyl)benzonitrile |
| 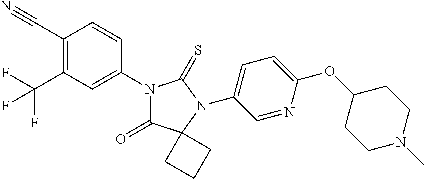 [SMILES: CN1CCC(Oc2ccc(N3C(=S)N(c4ccc(C#N)c(C(F)(F)F)c4)C(=O)C34CCC4)cn2)CC1] | 14 | 4-(5-(6-((1-Methylpiperidin-4- yl)oxy)pyridin-3-yl)-8-oxo-6- thioxo-5,7-diazaspiro[3.4]octan- 7-yl)-2- (trifluoromethyl)benzonitrile |
| 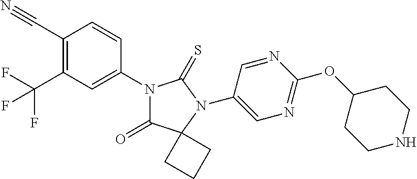 [SMILES: N#Cc1ccc(N2C(=O)C3(CCC3)N(c3cnc(OC4CCNCC4)nc3)C2=S)cc1C(F)(F)F] | 15 | 4-(8-Oxo-5-(2-(piperidin-4- yloxy)pyrimidin-5-yl)-6-thioxo- 5,7-diazaspiro[3.4]octan-7-yl)-2- (trifluoromethyl)benzonitrile |
| 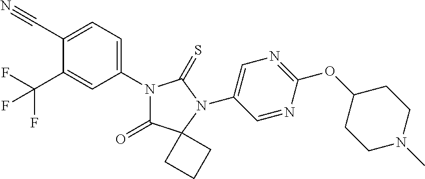 [SMILES: CN1CCC(Oc2ncc(N3C(=S)N(c4ccc(C#N)c(C(F)(F)F)c4)C(=O)C34CCC4)cn2)CC1] | 16 | 4-(5-(2-((1-Methylpiperidin-4- yl)oxy)pyrimidin-5-yl)-8-oxo-6- thioxo-5,7-diazaspiro[3.4]octan- 7-yl)-2- (trifluoromethyl)benzonitrile |
| 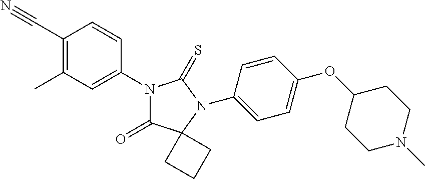 [SMILES: Cc1cc(N2C(=O)C3(CCC3)N(c3ccc(OC4CCN(C)CC4)cc3)C2=S)ccc1C#N] | 17 | 2-Methyl-4-(5-(4-((1- methylpiperidin-4- yl)oxy)phenyl)-8-oxo-6-thioxo- 5,7-diazaspiro[3.4]octan-7- yl)benzonitrile |
| 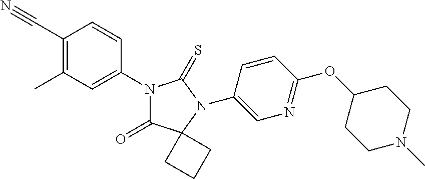 [SMILES: Cc1cc(N2C(=O)C3(CCC3)N(c3ccc(OC4CCN(C)CC4)nc3)C2=S)ccc1C#N] | 18 | 2-Methyl-4-(5-(6-((1- methylpiperidin-4- yl)oxy)pyridin-3-yl)-8-oxo-6- thioxo-5,7-diazaspiro[3.4]octan- 7-yl)benzonitrile |
| 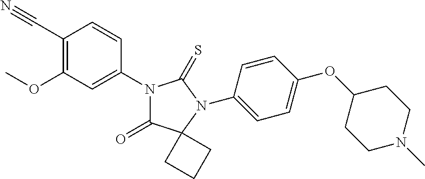 [SMILES: COc1cc(N2C(=O)C3(CCC3)N(c3ccc(OC4CCN(C)CC4)cc3)C2=S)ccc1C#N] | 19 | 2-Methoxy-4-(5-(4-((1- methylpiperidin-4- yl)oxy)phenyl)-8-oxo-6-thioxo- 5,7-diazaspiro[3.4]octan-7- yl)benzonitrile |
| 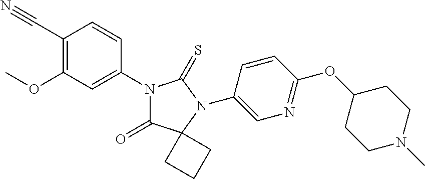 [SMILES: COc1cc(N2C(=O)C3(CCC3)N(c3ccc(OC4CCN(C)CC4)nc3)C2=S)ccc1C#N] | 20 | 2-Methoxy-4-(5-(6-((1- methylpiperidin-4- yl)oxy)pyridin-3-yl)-8-oxo-6- thioxo-5,7-diazaspiro[3.4]octan- 7-yl)benzonitrile |
| 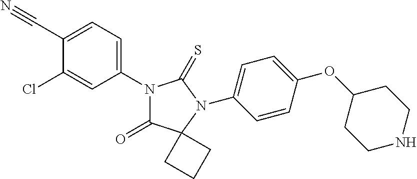 [SMILES: N#Cc1ccc(N2C(=O)C3(CCC3)N(c3ccc(OC4CCNCC4)cc3)C2=S)cc1Cl] | 21 | 2-Chloro-4-(8-oxo-5-(4- (piperidin-4-yloxy)phenyl)-6- thioxo-5,7-diazaspiro[3.4]octan- 7-yl)benzonitrile |
| 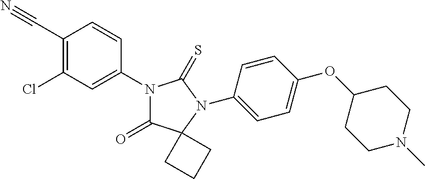 [SMILES: CN1CCC(Oc2ccc(N3C(=S)N(c4ccc(C#N)c(Cl)c4)C(=O)C34CCC4)cc2)CC1] | 22 | 2-Chloro-4-(5-(4-((1- methylpiperidin-4- yl)oxy)phenyl)-8-oxo-6-thioxo- 5,7-diazaspiro[3.4]octan-7- yl)benzonitrile |
| 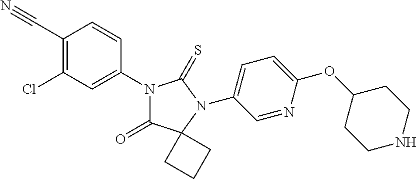 [SMILES: N#Cc1ccc(N2C(=O)C3(CCC3)N(c3ccc(OC4CCNCC4)nc3)C2=S)cc1Cl] | 23 | 2-Chloro-4-(8-oxo-5-(6- (piperidin-4-yloxy)pyridin-3-yl)- 6-thioxo-5,7- diazaspiro[3.4]octan-7- yl)benzonitrile |
| 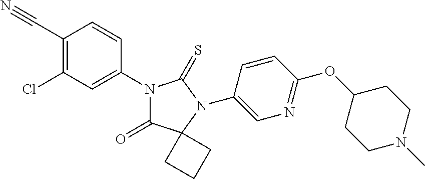 [SMILES: CN1CCC(Oc2ccc(N3C(=S)N(c4ccc(C#N)c(Cl)c4)C(=O)C34CCC4)cn2)CC1] | 24 | 2-Chloro-4-(5-(6-((1- methylpiperidin-4- yl)oxy)pyridin-3-yl)-8-oxo-6- thioxo-5,7-diazaspiro[3.4]octan- 7-yl)benzonitrile |

In a further embodiment, the invention is directed to a compound of Formula (I)

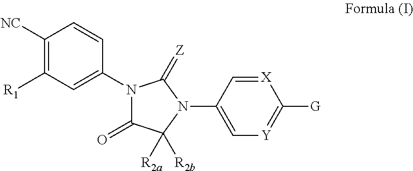

selected from the group consisting of
Cpd 1, 5-[8-[6-[(1-methyl-4-piperidyl)oxy]-3-pyridyl]-5-oxo-7-thioxo-6,8-diazaspiro[3.4]octan-6-yl]-3-(trifluoromethyl)pyridine-2-carbonitrile;
Cpd 2, 3-methyl-5-[8-[6-[(1-methyl-4-piperidyl)oxy]-3-pyridyl]-5-oxo-7-thioxo-6,8-diazaspiro[3.4]octan-6-yl]pyridine-2-carbonitrile;
Cpd 3, 4-(4,4-Dimethyl-5-oxo-3-(6-(piperidin-4-yloxy)pyridin-3-yl)-2-thioxoimidazolidin-1-yl)-2-(trifluoromethyl)benzonitrile;
Cpd 4, 4-(4,4-Dimethyl-3-(6-((1-methylpiperidin-4-yl)oxy)pyridin-3-yl)-5-oxo-2-thioxoimidazolidin-1-yl)-2-(trifluoromethyl)benzonitrile;
Cpd 5, 4-[4,4-Dimethyl-5-oxo-3-[2-(4-piperidinyloxy)pyrimidin-5-yl]-2-thioxo-imidazolidin-1-yl]-2-(trifluoromethyl)benzonitrile;
Cpd 6, 4-(4,4-Dimethyl-3-(2-((1-methylpiperidin-4-yl)oxy)pyrimidin-5-yl)-5-oxo-2-thioxoimidazolidin-1-yl)-2-(trifluoromethyl)benzonitrile;
Cpd 7, 2-Chloro-4-(4,4-dimethyl-5-oxo-3-(4-(piperidin-4-yloxy)phenyl)-2-thioxoimidazolidin-1-yl)benzonitrile;
Cpd 8, 2-Chloro-4-(4,4-dimethyl-3-(4-((1-methylpiperidin-4-yl)oxy)phenyl)-5-oxo-2-thioxoimidazolidin-1-yl)benzonitrile;
Cpd 9, 2-Chloro-4-(4,4-dimethyl-5-oxo-3-(6-(piperidin-4-yloxy)pyridin-3-yl)-2-thioxoimidazolidin-1-yl)benzonitrile hydrochloride;
Cpd 10, 2-Chloro-4-(4,4-dimethyl-3-(6-((1-methylpiperidin-4-yl)oxy)pyridin-3-yl)-5-oxo-2-thioxoimidazolidin-1-yl)benzonitrile;
Cpd 11, 4-(8-Oxo-5-(4-(piperidin-4-yloxy)phenyl)-6-thioxo-5,7-diazaspiro[3.4]octan-7-yl)-2-(trifluoromethyl)benzonitrile;
Cpd 12, 4-(5-(4-((1-Methylpiperidin-4-yl)oxy)phenyl)-8-oxo-6-thioxo-5,7-diazaspiro[3.4]octan-7-yl)-2-(trifluoromethyl)benzonitrile;
Cpd 13, 4-(8-Oxo-5-(6-(piperidin-4-yloxy)pyridin-3-yl)-6-thioxo-5,7-diazaspiro[3.4]octan-7-yl)-2-(trifluoromethyl)benzonitrile;
Cpd 14, 4-(5-(6-((1-Methylpiperidin-4-yl)oxy)pyridin-3-yl)-8-oxo-6-thioxo-5,7-diazaspiro[3.4]octan-7-yl)-2-(trifluoromethyl)benzonitrile;
Cpd 15, 4-(8-Oxo-5-(2-(piperidin-4-yloxy)pyrimidin-5-yl)-6-thioxo-5,7-diazaspiro[3.4]octan-7-yl)-2-(trifluoromethyl)benzonitrile;
Cpd 16, 4-(5-(2-((1-Methylpiperidin-4-yl)oxy)pyrimidin-5-yl)-8-oxo-6-thioxo-5,7-diazaspiro[3.4]octan-7-yl)-2-(trifluoromethyl)benzonitrile;
Cpd 17, 2-Methyl-4-(5-(4-((1-methylpiperidin-4-yl)oxy)phenyl)-8-oxo-6-thioxo-5,7-diazaspiro[3.4]octan-7-yl)benzonitrile;
Cpd 18, 2-Methyl-4-(5-(6-((1-methylpiperidin-4-yl)oxy)pyridin-3-yl)-8-oxo-6-thioxo-5,7-diazaspiro[3.4]octan-7-yl)benzonitrile;
Cpd 19, 2-Methoxy-4-(5-(4-((1-methylpiperidin-4-yl)oxy)phenyl)-8-oxo-6-thioxo-5,7-diazaspiro[3.4]octan-7-yl)benzonitrile;
Cpd 20, 2-Methoxy-4-(5-(6-((1-methylpiperidin-4-yl)oxy)pyridin-3-yl)-8-oxo-6-thioxo-5,7-diazaspiro[3.4]octan-7-yl)benzonitrile;
Cpd 21, 2-Chloro-4-(8-oxo-5-(4-(piperidin-4-yloxy)phenyl)-6-thioxo-5,7-diazaspiro[3.4]octan-7-yl)benzonitrile;
Cpd 22, 2-Chloro-4-(5-(4-((1-methylpiperidin-4-yl)oxy)phenyl)-8-oxo-6-thioxo-5,7-diazaspiro[3.4]octan-7-yl)benzonitrile;
Cpd 23, 2-Chloro-4-(8-oxo-5-(6-(piperidin-4-yloxy)pyridin-3-yl)-6-thioxo-5,7-diazaspiro[3.4]octan-7-yl)benzonitrile;
and
Cpd 24, 2-Chloro-4-(5-(6-((1-methylpiperidin-4-yl)oxy)pyridin-3-yl)-8-oxo-6-thioxo-5,7-diazaspiro[3.4]octan-7-yl)benzonitrile;
or a pharmaceutically acceptable salt form thereof.

For use in medicine, salts of compounds of Formula (I) refer to non-toxic “pharmaceutically acceptable salts.” Other salts may, however, be useful in the preparation of compounds of Formula (I) or of their pharmaceutically acceptable salt forms thereof. Suitable pharmaceutically acceptable salts of compounds of Formula (I) include acid addition salts that can, for example, be formed by mixing a solution of the compound with a solution of a pharmaceutically acceptable acid such as, hydrochloric acid, sulfuric acid, fumaric acid, maleic acid, succinic acid, acetic acid, benzoic acid, citric acid, tartaric acid, carbonic acid or phosphoric acid. Furthermore, where the compounds of Formula (I) carry an acidic moiety, suitable pharmaceutically acceptable salts thereof may include alkali metal salts such as, sodium or potassium salts; alkaline earth metal salts such as, calcium or magnesium salts; and salts formed with suitable organic ligands such as, quaternary ammonium salts. Thus, representative pharmaceutically acceptable salts include acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide, and valerate.

Representative acids and bases that may be used in the preparation of pharmaceutically acceptable salts include acids including acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hippuric acid, hydrobromic acid, hydrochloric acid, (+)-L-lactic acid, (+)-DL-lactic acid, lactobionic acid, maleic acid, (−)-L-malic acid, malonic acid, (+)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid; and bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, sodium hydroxide, triethanolamine, tromethamine, and zinc hydroxide.

Embodiments of the present invention include prodrugs of compounds of Formula (I). In general, such prodrugs will be functional derivatives of the compounds that are readily convertible in vivo into the required compound. Thus, in the methods of treating or preventing embodiments of the present invention, the term “administering” encompasses the treatment or prevention of the various diseases, conditions, syndromes and disorders described with the compound specifically disclosed or with a compound that may not be specifically disclosed, but which converts to the specified compound in vivo after administration to a patient. Conventional procedures for the selection and preparation of suitable prodrug derivatives are described, for example, in “Design of Prodrugs”, ed. H. Bundgaard, Elsevier, 1985.

Where the compounds according to embodiments of this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention. The skilled artisan will understand that the term compound as used herein, is meant to include solvated compounds of Formula (I).

Where the processes for the preparation of the compounds according to certain embodiments of the invention give rise to mixture of stereoisomers, these isomers may be separated by conventional techniques such as, preparative chromatography. The compounds may be prepared in racemic form, or individual enantiomers may be prepared either by enantiospecific synthesis or by resolution. The compounds may, for example, be resolved into their component enantiomers by standard techniques such as, the formation of diastereomeric pairs by salt formation with an optically active acid such as, (−)-di-p-toluoyl-d-tartaric acid and/or (+)-di-p-toluoyl-l-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

One embodiment of the present invention is directed to a composition, including a pharmaceutical composition, comprising, consisting of, and/or consisting essentially of the (+)-enantiomer of a compound of Formula (I) wherein said composition is substantially free from the (−)-isomer of said compound. In the present context, substantially free means less than about 25%, preferably less than about 10%, more preferably less than about 5%, even more preferably less than about 2% and even more preferably less than about 1% of the (−)-isomer calculated as

$$\% ( + ) - enantiomer = \frac{( mass ( + ) - enantiomer )}{( mass ( + ) - enantiomer ) + ( mass ( - ) - enantiomer )} \times 100.$$

Another embodiment of the present invention is a composition, including a pharmaceutical composition, comprising, consisting of, and consisting essentially of the (−)-enantiomer of a compound of Formula (I) wherein said composition is substantially free from the (+)-isomer of said compound. In the present context, substantially free from means less than about 25%, preferably less than about 10%, more preferably less than about 5%, even more preferably less than about 2% and even more preferably less than about 1% of the (+)-isomer calculated as

$$\% ( + ) - enantiomer = \frac{( mass ( - ) - enantiomer )}{( mass ( + ) - enantiomer ) + ( mass ( - ) - enantiomer )} \times 100.$$

During any of the processes for preparation of the compounds of the various embodiments of the present invention, it may be necessary and/or desirable to protect sensitive or reactive groups on any of the molecules concerned. This may be achieved by means of conventional protecting groups such as those described in Protective Groups in Organic Chemistry, Second Edition, J. F. W. McOmie, Plenum Press, 1973; T. W. Greene & P. G. M. Wuts, Protective Groups in Organic Synthesis, John Wiley & Sons, 1991; and T. W. Greene & P. G. M. Wuts, Protective Groups in Organic Synthesis, Third Edition, John Wiley & Sons, 1999. The protecting groups may be removed at a convenient subsequent stage using methods known from the art.

Even though the compounds of embodiments of the present invention (including their pharmaceutically acceptable salts and pharmaceutically acceptable solvates) can be administered alone, they will generally be administered in admixture with a pharmaceutically acceptable carrier, a pharmaceutically acceptable excipient and/or a pharmaceutically acceptable diluent selected with regard to the intended route of administration and standard pharmaceutical or veterinary practice. Thus, particular embodiments of the present invention are directed to pharmaceutical and veterinary compositions comprising compounds of Formula (I) and at least one pharmaceutically acceptable carrier, pharmaceutically acceptable excipient, and/or pharmaceutically acceptable diluent.

By way of example, in the pharmaceutical compositions of embodiments of the present invention, the compounds of Formula (I) may be admixed with any suitable binder(s), lubricant(s), suspending agent(s), coating agent(s), solubilizing agent(s), and combinations thereof.

Solid oral dosage forms such as, tablets or capsules, containing the compounds of the present invention may be administered in at least one dosage form at a time, as appropriate. It is also possible to administer the compounds in sustained release formulations.

Additional oral forms in which the present inventive compounds may be administered include elixirs, solutions, syrups, and suspensions; each optionally containing flavoring agents and coloring agents.

Alternatively, compounds of Formula (I) can be administered by inhalation (intratracheal or intranasal) or in the form of a suppository or pessary, or they may be applied topically in the form of a lotion, solution, cream, ointment or dusting powder. For example, they can be incorporated into a cream comprising, consisting of, and/or consisting essentially of an aqueous emulsion of polyethylene glycols or liquid paraffin. They can also be incorporated, at a concentration of between about 1% and about 10% by weight of the cream, into an ointment comprising, consisting of, and/or consisting essentially of a wax or soft paraffin base together with any stabilizers and preservatives as may be required. An alternative means of administration includes transdermal administration by using a skin or transdermal patch.

The pharmaceutical compositions of the present invention (as well as the compounds of the present invention alone) can also be injected parenterally, for example, intracavernosally, intravenously, intramuscularly, subcutaneously, intradermally, or intrathecally. In this case, the compositions will also include at least one of a suitable carrier, a suitable excipient, and a suitable diluent.

For parenteral administration, the pharmaceutical compositions of the present invention are best used in the form of a sterile aqueous solution that may contain other substances, for example, enough salts and monosaccharides to make the solution isotonic with blood.

For buccal or sublingual administration, the pharmaceutical compositions of the present invention may be administered in the form of tablets or lozenges, which can be formulated in a conventional manner.

By way of further example, pharmaceutical compositions containing at least one of the compounds of Formula (I) as the active ingredient can be prepared by mixing the compound(s) with a pharmaceutically acceptable carrier, a pharmaceutically acceptable diluent, and/or a pharmaceutically acceptable excipient according to conventional pharmaceutical compounding techniques. The carrier, excipient, and diluent may take a wide variety of forms depending upon the desired route of administration (e.g., oral, parenteral, etc.). Thus, for liquid oral preparations such as, suspensions, syrups, elixirs and solutions, suitable carriers, excipients and diluents include water, glycols, oils, alcohols, flavoring agents, preservatives, stabilizers, coloring agents and the like; for solid oral preparations such as, powders, capsules, and tablets, suitable carriers, excipients and diluents include starches, sugars, diluents, granulating agents, lubricants, binders, disintegrating agents and the like. Solid oral preparations also may be optionally coated with substances such as, sugars, or be enterically coated so as to modulate the major site of absorption and disintegration. For parenteral administration, the carrier, excipient and diluent will usually include sterile water, and other ingredients may be added to increase solubility and preservation of the composition. Injectable suspensions or solutions may also be prepared utilizing aqueous carriers along with appropriate additives such as, solubilizers and preservatives.

A therapeutically effective amount of a compound of Formula (I) or a pharmaceutical composition thereof includes a dose range from about 0.1 mg to about 3000 mg, or any particular amount or range therein, in particular from about 1 mg to about 1000 mg, or any particular amount or range therein, or, more particularly, from about 10 mg to about 500 mg, or any particular amount or range therein, of active ingredient in a regimen of about 1 to about 4 times per day for an average (70 kg) human; although, it is apparent to one skilled in the art that the therapeutically effective amount for a compound of Formula (I) will vary as will the diseases, syndromes, conditions, and disorders being treated.

For oral administration, a pharmaceutical composition is preferably provided in the form of tablets containing about 1.0, about 10, about 50, about 100, about 150, about 200, about 250, and about 500 milligrams of a compound of Formula (I).

An embodiment of the present invention is directed to a pharmaceutical composition for oral administration, comprising a compound of Formula (I) in an amount of from about 25 mg to about 500 mg.

Advantageously, a compound of Formula (I) may be administered in a single daily dose, or the total daily dosage may be administered in divided doses of two, three and four times daily.

Optimal dosages of a compound of Formula (I) to be administered may be readily determined and will vary with the particular compound used, the mode of administration, the strength of the preparation and the advancement of the disease, syndrome, condition or disorder. In addition, factors associated with the particular subject being treated, including subject gender, age, weight, diet and time of administration, will result in the need to adjust the dose to achieve an appropriate therapeutic level and desired therapeutic effect. The above dosages are thus exemplary of the average case. There can be, of course, individual instances wherein higher or lower dosage ranges are merited, and such are within the scope of this invention.

Compounds of Formula (I) may be administered in any of the foregoing compositions and dosage regimens or by means of those compositions and dosage regimens established in the art whenever use of a compound of Formula (I) is required for a subject in need thereof.

One embodiment of the present invention is directed to a pharmaceutical composition comprising a compound selected from the group consisting of 4-(4,4-dimethyl-5-oxo-3-(6-(piperidin-4-yloxy)pyridin-3-yl)-2-thioxoimidazolidin-1-yl)-2-(trifluoromethyl)benzonitrile, 4-(4,4-dimethyl-3-(6-((1-methylpiperidin-4-yl)oxy)pyridin-3-yl)-5-oxo-2-thioxoimidazolidin-1-yl)-2-(trifluoromethyl)benzonitrile, 4-(8-oxo-5-(6-(piperidin-4-yloxy)pyridin-3-yl)-6-thioxo-5,7-diazaspiro[3.4]octan-7-yl)-2-(trifluoromethyl)benzonitrile, and 4-(5-(6-((1-methylpiperidin-4-yl)oxy)pyridin-3-yl)-8-oxo-6-thioxo-5,7-diazaspiro[3.4]octan-7-yl)-2-(trifluoromethyl)benzonitrile, and at least one of a pharmaceutically acceptable carrier, a pharmaceutically acceptable excipient, and a pharmaceutically acceptable diluent.

In another embodiment of the present invention, the compounds and compositions, according to the method of the present invention, may be administered using any amount and any route of administration effective for treating a cancer or another proliferative disease, disorder or condition. In some embodiments, the cancer or other proliferative disease, disorder or condition is a prostate cancer.

In some embodiments, the cancer or other proliferative disease, disorder or condition is a castration-resistant prostate cancer (CRPC). In some embodiments, the cancer or other proliferative disease, disorder or condition is a castration-resistant prostate cancer (CRPC) bearing a mutation in AR. In some embodiments, the mutation in AR is a mutation of Phenylalanine (Phe)876.

In some embodiments, the mutation in AR is a mutation of Phe876 to leucine. In some embodiments, the mutation in AR is a mutation of Phe876 to isoleucine. In some embodiments, the mutation in AR is a mutation of Phe876 to valine. In some embodiments, the mutation in AR is a mutation of Phe876 to serine. In some embodiments, the mutation in AR is a mutation of Phe876 to cysteine. In some embodiments, the mutation in AR is a mutation of Phe876 to tyrosine.

In some embodiments, the cancer or other proliferative disease, disorder or condition is a prostate cancer that is resistant to any AR therapy as a consequence of mutation.

In some embodiments, the cancer or other proliferative disease, disorder or condition is a prostate cancer that is resistant to treatment using second-generation AR antagonists, including, but not limited to, Enzalutamide or ARN-509.

The present invention encompasses the recognition that mutations in the AR polypeptide can render the AR polypeptide resistant to anti-androgens or convert anti-androgens to androgen agonists. In some embodiments, the present invention provides compounds that can be used to effect anti-androgenic effects despite the presence of such mutations.

The amino acid sequence of an AR polypeptide described herein can exist in a mutant AR containing, or can be modified to produce an mutant AR polypeptide variant at least one (e.g., 1, 2, 3, 4, 5, 6, 7, 8, 9, 10, or more) additions, substitutions, or deletions of a wild-type amino acid residue.

In some embodiments, the AR polypeptide variants described herein result in a loss of inhibition of AR activity by one or more antiandrogens of 0, 1, 2, 3, 4, 5, 6, 7, 8, 9, 10 up to 100%. In some embodiments, the AR polypeptide variants described herein convert antiandrogens to androgen receptor agonists.

Specific, nonlimiting amino acid residues that can be modified in an AR mutant include, e.g., E566, E589, E669, C687, A700, N772, H777, C785, F877, K911, of the AR polypeptide. These amino acid residues can be substituted with any amino acid or amino acid analog. For example, the substitutions at the recited positions can be made with any of the naturally-occurring amino acids (e.g., alanine, aspartic acid, asparagine, arginine, cysteine, glycine, glutamic acid, glutamine, histidine, leucine, valine, isoleucine, lysine, methionine, proline, threonine, serine, phenylalanine, tryptophan, or tyrosine). In particular instances, an amino acid substitution is E566K, E589K, E669K, C687Y, A700T, N772S, H777Y, C785R, F877C, F877I, F877L, F877S, F877V, F877Y and/or K911E.

In some embodiments, the AR mutants as described herein can include additional modifications of the AR polypeptide previously described in the art, including but not limited to, e.g., A597T, S648G, P683T, D696E, R727H, N728I, 1738F, W741L, W741C, W741L, M743V, G751S, A871V, H874Y, T878A, T878S, and P914S.

In some embodiments, the compounds and compositions, according to the method of the present invention, may be administered using any amount and any route of administration effective for treating a bone disease, disorder or condition. In some embodiments, the bone disease, disorder or condition is osteoporosis.

In some embodiments, the present invention is directed to a compound of Formula (I) for use in the treatment of a disease, a syndrome, a condition or a disorder in a subject, including an animal, a mammal and a human in which the disease, the syndrome, the condition or the disorder is affected by the antagonism of the androgen receptor, selected from the group consisting of prostate cancer, castration-resistant prostate cancer, and metastatic castration-resistant prostate cancer.

In certain embodiments, a compound of Formula (I), or a composition thereof, may be administered in combination with another modulator, agonist or antagonist of AR. In some embodiments, the compound of Formula (I), or composition thereof, may be administered in combination with one or more other therapeutic agents.

In some embodiments the AR modulators, agonists or antagonists include, but are not limited to gonadotropin-releasing hormone agonists or antagonists (e.g. Lupron, Zoladex (Goserelin), Degarelix, Ozarelix, ABT-620 (Elagolix), TAK-385 (Relugolix), EP-100 or KLH-2109); non-steroidal antiandrogens, aminoglutethimide, enzalutamide, bicalutamide, nilutamide, flutamide, steroidal antiandrogens, finasteride, dutasteride, bexlosteride, izonsteride, turosteride, epristeride, other inhibitors of 5-alphareductase, 3,3′-diindolylmethane (DIM), N-butylbenzene-sulfonamide (NBBS); or a CYP17 inhibitor such as abiraterone acetate, TAK-700 (orteronel), TOK-001 (galeterone) or VT-464.

A further embodiment of the present invention is directed to a pharmaceutical composition comprising, consisting of, and/or consisting essentially of a compound of Formula (I) and a therapeutically effective amount of abiraterone acetate.

A further embodiment of the present invention is directed to a pharmaceutical composition comprising, consisting of, and/or consisting essentially of a compound of Formula (I) and abiraterone acetate and, optionally, prednisone or dexamethasone.

In certain embodiments, a compound of Formula (I), or a pharmaceutical composition thereof, may be administered in combination with a PI3K pathway inhibitor.

In some embodiments the PI3K pathway inhibitors (PI3K, TORC or dual PI3K/TORC inhibitor) include, but are not limited to, everolimus, BEZ-235, BKM120, BGT226, BYL-719, GDC0068, GDC-0980, GDC0941, GDC0032, MK-2206, OSI-027, CC-223, AZD8055, SAR245408, SAR245409, PF04691502, WYE125132, GSK2126458, GSK-2636771, BAY806946, PF-05212384, SF1126, PX866, AMG319, ZSTK474, CallOl, PWT33597, LY-317615 (enzastaurin hydrochloride), CU-906, or CUDC-907.

In certain embodiments, a compound of Formula (I), or a composition thereof, may be administered in combination with radiation therapy. The term “radiotherapy” or “ionizing radiation” include all forms of radiation, including but not limited to α, β, and γ radiation and ultraviolet light.

In some embodiments radiation therapy includes, but is not limited to, radioactive implants directly inserted in a tumor or body cavity (brachytherapy, interstitial irradiation, and intracavitary irradiation are types of internal radiotherapy), radiopharmaceuticals (e.g. Alpharadin (Radium-223 Chloride), 177Lu-J591 PSMA conjugate), or external beam radiation therapy (including Proton beam).

In certain embodiments, a compound of Formula (I), or a pharmaceutical composition thereof, may be administered in combination with immunotherapy.

In some embodiments the immunotherapy includes, but is not limited to Provenge, Prostvac, Ipilimumab, a CTLA-4 inhibitor or a PD-1 inhibitor.

### General Synthetic Methods

Representative compounds of the present invention can be synthesized in accordance with the general synthetic methods described below and illustrated in the schemes and examples that follow. Since the schemes are an illustration, the invention should not be construed as being limited by the chemical reactions and conditions described in the schemes and examples. Compounds analogous to the target compounds of these examples can be made according to similar routes. The disclosed compounds are useful as pharmaceutical agents as described herein. The various starting materials used in the schemes and examples are commercially available or may be prepared by methods well within the skill of persons versed in the art.

Abbreviations used in the instant specification, particularly the schemes and examples, are as follows:
ACN acetonitrile
AcOH acetic acid
AIBN 2,2′-azobisisobutyronitrile
Boc tert-butyl carbamate
BOP benzotriazol-1-yloxy)tris(dimethylamino)phosphonium hexfluorophosphate
BuLi butyllithium
Cbz benzyl carbamate
CSS Charcoal Stripped Serum
DIBAL-H diisobutylaluminum hydride
DBU 1.8-diazabicyclo[5.4.0]undec-7-ene
DCC N,N′-dicyclohexylcarbodiimide
DCE 1,2-dichloroethane
DCM dichloromethane
DEAD diethyl azodicarboxylate
DIAD diisopropyl azodicarboxylate
DIPEA diisopropylethylamine
DMA dimethylacetamide
DMAP 4-(dimethylamino)pyridine
DME ethylene glycol dimethyl ether
DMEM Dulbecco's Modified Eagle's Medium
DMF dimethylformamide
DMSO dimethyl sulfoxide
EA ethyl acetate
EDC N-(3-dimethylaminopropyl)-N′-ethylcarbodiimide
EDCI 1-(3-dimethylaminopropyl)-3-ethylcarbodiimide hydrochloride
EMEM Eagle's Minimum Essential Medium
Et ethyl
Et₂O diethyl ether
EtOAc ethyl acetate
EtOH ethyl alcohol
FCS Fetal Calf Serum
h or hr hour(s)
HATU O-(7-azabenzotriazol-1-yl)-N,N,N′,N′-tetramethyluronium hexafluorophosphate
HCHO formaldehyde
HCl hydrochloric acid
HCOOH formic acid
HMPA hexamethylphosphoramide
HOBt 1-hydroxybenzotriazole monohydrate
HPLC high performance liquid chromatography
KCN potassium cyanide
LCMS high pressure liquid chromatography with mass spectrometer
LDA lithium diisopropylamide
LiOH lithium hydroxide
LHMDS lithium hexamethyl disilazide
Me methyl
MeCN acetonitrile
MeOH methyl alcohol
mg milligram
min minute
MOM methoxymethyl
NaCN sodium cyanide
NaHMDS sodium hexamethyl disilazide
NaOH sodium hydroxide
NaO^{t}Bu sodium tert-butoxide
NBS N-bromosuccinimide
NH₄Cl ammonium chloride
NMP N-methyl pyrrolidinone
N,N-DMA N, N-dimethylacetamide
PBS Phosphate Buffered Saline
Pd/C palladium on charcoal
Pd₂(dba)₃ tris(dib enzylideneacetnedipaladiu
Pd(dppf)Cl₂[1,1′-bis(diphenylphosphino)ferrocene]dichloropalladium
Pd(OAc)₂ palladium diacetate
Pd(PPh₃)₄ tetrakis(triphenylphosphine)palladium
PPh₃ triphenyl phosphine
p-TsOH para-toluenesulfonic acid
RPMI Roswell Park Memorial Institute medium
rt or RT room temperature
SPE solid phase extraction
TBAF tetrabutyl ammonium fluoride
TBDMSCl tert-butyldimethylsilyl chloride
TBTU O-benzotriazol-1-yl-N,N,N′,N′-tetramethyluronium hexafluorophosphate
t-Bu tert-butyl
TEMPO 2,2,6,6-tetramethyl-1-piperdinyloxy, free radical
TFA trifluoroacetic acid
THF tetrahydrofuran
TLC thin layer chromatography
TMS-CN trimethylsilyl cyanide
TMSOTf trimethylsilyl trifluoromethanesulfonate

Compounds of Formula (I) may be prepared according to the process outlined in Scheme 1, below.

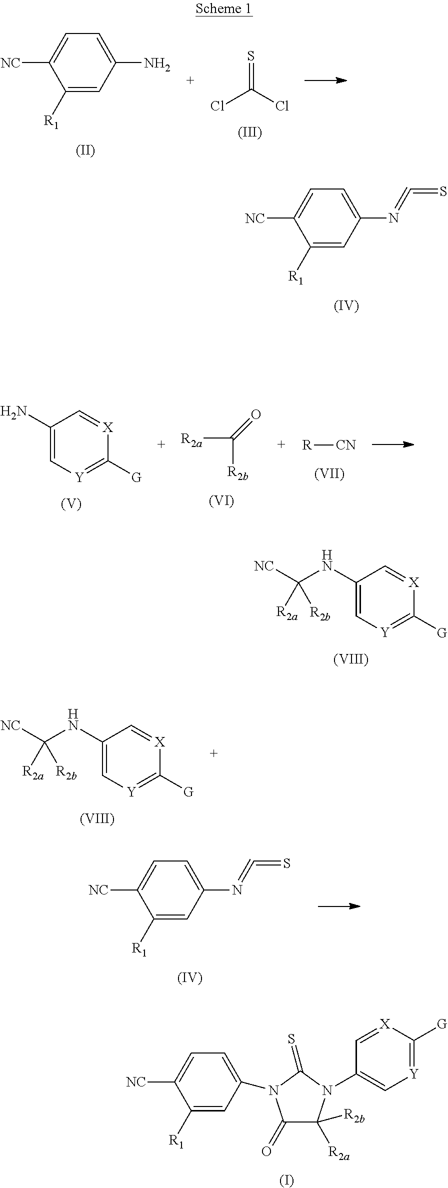

Accordingly, a suitably substituted compound of formula (II), a known compound or compound prepared by known methods, may be reacted with thiophosgene (III), phenyl chlorothionocarbonate, in the presence of a suitably selected base such as DMAP, K₂CO₃, Cs₂CO₃, and the like, in a suitably selected solvent or mixture of solvents such as CHCl₃, CH₂Cl₂, 1,2-dichloroethane, water, THF, toluene, and the like, at temperature ranging from about 0 to about 130° C., to yield the corresponding compound of formula (IV). A suitably substituted compound of formula (V), a known compound or compound prepared by known methods, wherein G is optionally substituted heterocyclyl, may be reacted with a compound of formula (VI), a known compound or compound prepared by known methods, in the presence of a suitably selected source of cyanide (VII), such as KCN, NaCN, TMS-CN, and the like; in a suitably selected solvent or mixture of solvents such as acetic acid, EtOH, MeO, and the like, at temperature ranging from about 10 to about 130° C., to yield the corresponding compound of formula (VIII).

The compound of formula (IV) may then be reacted with the compound of formula (VIII) in a suitably selected solvent or mixture of solvents such as DMA, DMF, NMP, DSMO, and the like, at temperature ranging from about 15 to about 180° C., to yield the corresponding compound of formula (I).

Compounds of formula (I) may alternatively be prepared according to the process as outlined in Scheme 2, below.

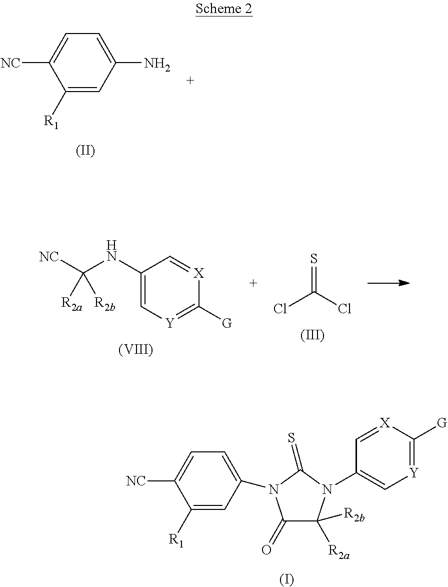

Alternatively, a suitably substituted compound of formula (II), a known compound or compound prepared by known methods, may be reacted with a compound of formula (VIII), a known compound or compound prepared by known methods, wherein G is optionally substituted heterocyclyl as defined herein, and thiophosgene, in the presence of a Lewis acid such as TMSOTf, AlCl₃, ZnCl₂, and the like, in a suitably selected solvent or mixture of solvents such as DMA, DMF, NMP, DSMO, and the like, at temperature ranging from about 0 to about 180° C., to yield the corresponding compound of formula (I).

Compounds of formula (I) may alternatively be prepared according to the process as outlined in Scheme 3, below.

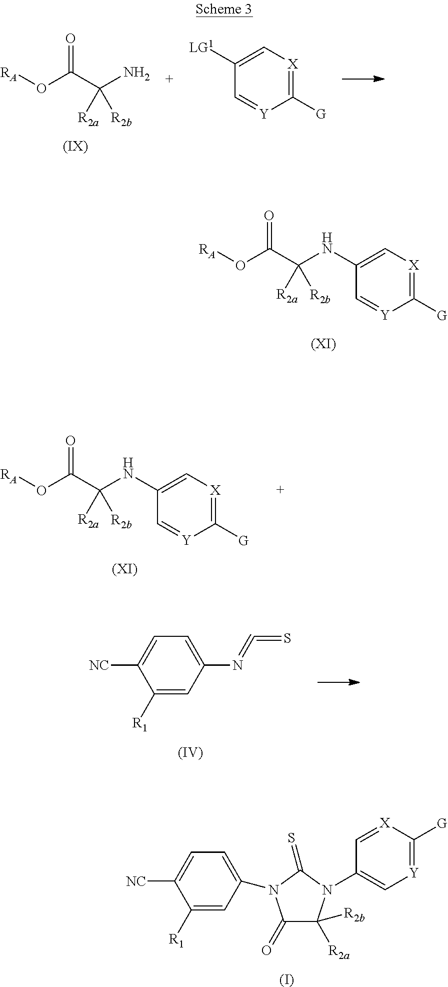

Alternatively, a suitably substituted compound of formula (IX), a known compound or compound prepared by known methods, wherein R_{A} is H, lower alkyl, and the like, may be reacted with a compound of formula (X), wherein LG¹ is a leaving group such as iodo, bromo, chloro, triflate, and the like, and G is optionally substituted heterocyclyl as defined herein, in the presence of a copper catalyst such as CuI, and the like, in the presence of a suitably selected base such as DBU, tBuOK, and the like; in a suitably selected solvent such as DMA, DMF, NMP, DMSO, and the like; at temperature ranging from about 15 to about 170° C., under Ullman coupling conditions, to yield the corresponding compound of formula (XI). The compound of formula (XI) may then be reacted with the compound of formula (IV) in a suitably selected solvent or mixture of solvents such as THF, 1,4-dioxane, toluene, DMSO, and the like, at temperature ranging from about 15 to about 180° C., to yield the corresponding compound of formula (I).

Compounds of formula (I) may alternatively be prepared according to the process as outlined in Scheme 4, below.

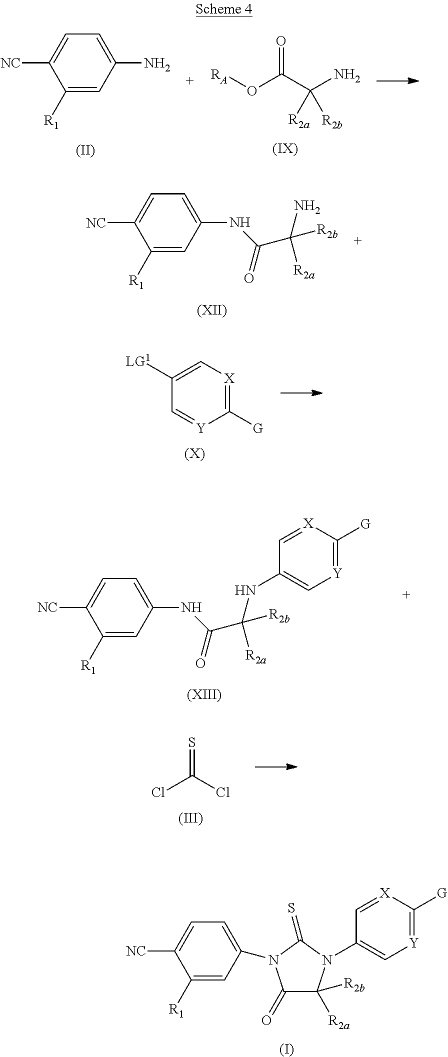

Alternatively, a suitably substituted compound of formula (II), a known compound or compound prepared by known methods, may be reacted with a compound of formula (IX), a known compound or compound prepared by known methods, wherein R_{A} is H, lower alkyl, and the like, to yield the corresponding compound of formula (XII). The compound of formula (XII) may then be reacted with the compound of formula (X), wherein LG¹ is a leaving group such as iodo, bromo, chloro, triflate, and the like, and G is optionally substituted heterocyclyl as defined herein, in the presence of a copper catalyst such as CuI, and the like, in the presence of a suitably selected base such as DBU, tBuOK, and the like; in a suitably selected solvent such as DMA, DMF, NMP, DMSO, and the like; at temperature ranging from about 15 to about 170° C., under Ullman coupling conditions, to yield the corresponding compound of formula (XIII). The compound of formula (XIII) may then be reacted with thiophosgene (III), phenyl chlorothionocarbonate, in the presence of a suitably selected base such as DMAP, K₂CO₃, Cs₂CO₃, and the like, in a suitably selected solvent or mixture of solvents such as CHCl₃, CH₂Cl₂, 1,2-dichloroethane, water, THF, toluene, and the like, at temperature ranging from about 0 to about 130° C., to yield the corresponding compound of formula (I).

Certain compounds of the present invention wherein substituent G is represented as

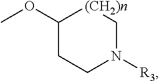

wherein n is an integer from 0 to 1, may be prepared according to the process outlined in Scheme 5, below.

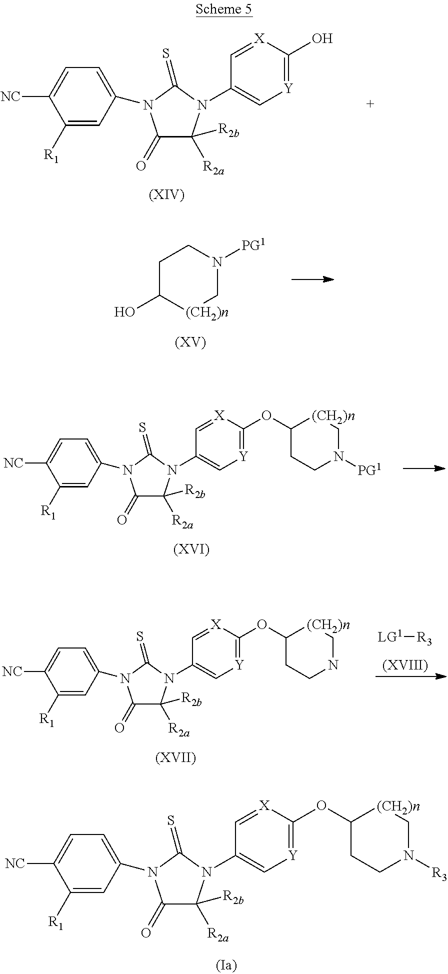

A suitably substituted compound of formula (XIV), a known compound or compound prepared by known methods, may be reacted with a suitably substituted compound of formula (XV) (wherein PG¹ is a suitably selected protecting group such as Boc, Cbz, and the like, and m and n are each independently an integer of 0 or 1), a known compound or compound prepared by known methods, in the presence of DIAD, DEAD, and the like, and PPh₃, under Mitsunobu conditions, in a suitably selected solvent or mixture of solvents such as THF, Et₂O, and the like; at temperature ranging from about 0 to about 130° C., to yield the corresponding compound of formula (XVI). The compound of formula (XVI) may then be deprotected under various conventional conditions, using reagents such as HCl or TFA when PG¹ is Boc, or hydrogenolysis when PG¹ is carboxybenzyl, for example, to afford the compound of formula (XVII).

The compound of formula (XVII) may then be reacted with a suitably selected compound of formula (XVIII), wherein LG¹ is a suitably selected leaving group, such as chloro, bromo, mesylate, tosylate, and the like, a known compound or compound prepared by known methods, in the presence of a base such as TEA, DIPEA, K₂CO₃, and the like in a suitable solvent such as DMF, DMSO, or MeCN, to yield the corresponding compound of formula (Ia).

Alternatively, the compound of formula (XVII) may be reacted with a suitably selected compound of formula (XVIII) wherein the compound (XVIII) includes an aldehyde or ketone carbonyl group, as would be readily recognized by one skilled in the art, under conventional reductive amination conditions, (for example, reacting with sodium triacetoxyborohydride and acetic acid, in a suitably selected solvent, such as DCM, DCE, THF, and the like; or reacting with sodium cyanoborohydride in a suitably selected solvent, such as methanol, and the like), to yield the corresponding compound of formula (Ia).

Alternatively, in some embodiments, G is

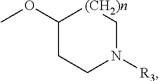

wherein n is an integer from 0 to 1, compounds disclosed herein may be prepared according to the process outlined in Scheme 6, below.

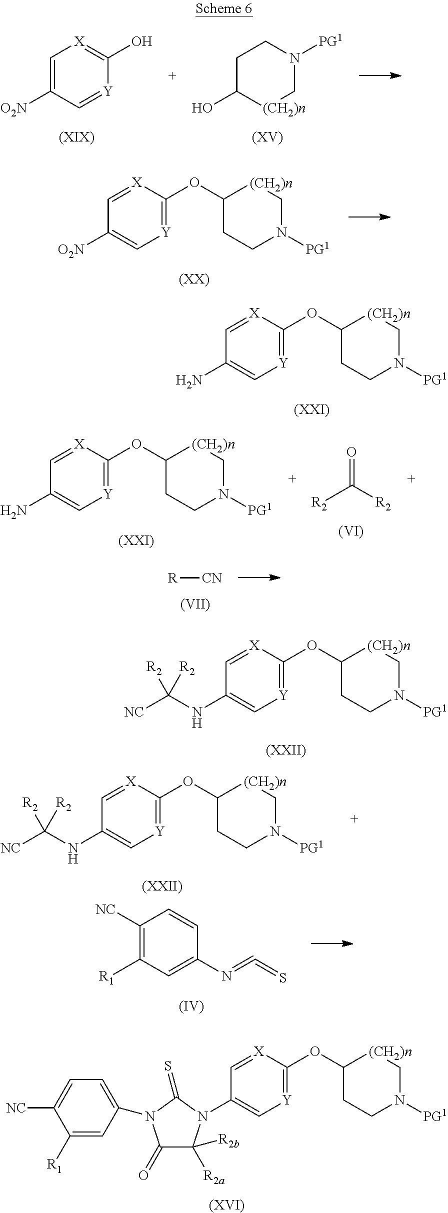

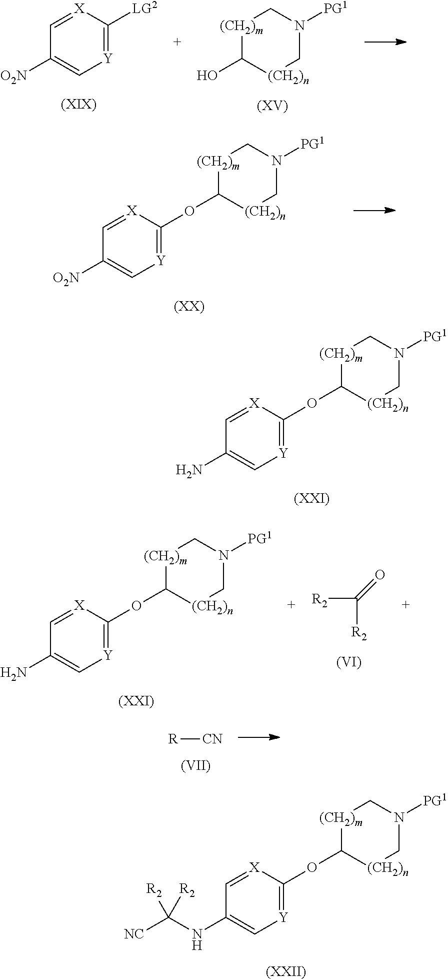

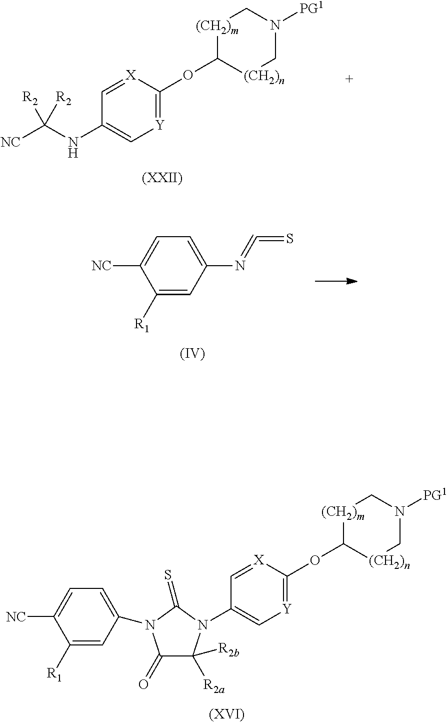

A suitably substituted compound of formula (XIX) wherein LG² is hydroxy, a known compound or compound prepared by known methods, may be reacted with a suitably substituted compound of formula (XV) wherein PG¹ is a suitably selected protecting group such as Boc, Cbz, and the like, a known compound or compound prepared by known methods, in the presence of DIAD, DEAD, and the like, and PPh₃, under Mitsunobu conditions, in a suitably selected solvent or mixture of solvents such as THF, Et₂O, and the like; at temperature ranging from about 0 to about 130° C., to yield the corresponding compound of formula (XX).

Alternatively, a suitably substituted compound of formula (XIX) wherein LG² is a leaving group such as iodo, bromo, chloro, triflate, and the like, may be reacted with a suitably substituted compound of formula (XV) wherein PG¹ is a suitably selected protecting group such as -Boc, -Cbz, and the like, a known compound or compound prepared by known methods, in the presence of a suitably selected base such as NaH, tBuOK, K₂CO₃, CsCO₃, DBU, and the like; in a suitably selected solvent such as THF, DMA, DMF, NMP, DMSO, and the like; at temperature ranging from about 15 to about 120° C., to yield the corresponding compound of formula (XX).

The compound of formula (XX) may then be reacted with a hydrogen source, under hydrogenation conditions, in the presence of a suitably selected catalysts or a catalyst system, such as Pd/C, Pt, and the like, in a suitably selected solvent such as MeOH, EtOAc, and the like, to yield the corresponding compound of formula (XXI). The compound of formula (XXI) may then be reacted with a compound of formula (VI), a known compound or compound prepared by known methods, in the presence of a suitably selected source of cyanide (VII), such as KCN, NaCN, TMS-CN, and the like; in a suitably selected solvent or mixture of solvents such as acetic acid, EtOH, MeOH, and the like; at temperature ranging from about 10 to about 130° C., to yield the corresponding compound of formula (XXII).

The compound of formula (XXII) may then be reacted with the compound of formula (IV) in a suitably selected solvent or mixture of solvents such as DMA, DMF, NMP, DSMO, and the like, at temperature ranging from about 15 to about 180° C., to yield the corresponding compound of formula (XVI). The compound of formula (XVI) may be further reacted as described in Scheme 5 to yield the corresponding compound of formula (Ia).

One of skill in the art will recognize that the nitro groups in compounds of formulas (XIX) and (XX) may be substituted with a suitably protected amine function that may be subsequently deprotected to yield the amine of formula (XXI) following the Mitsunobu reaction.

### SPECIFIC EXAMPLES

In the following Examples, some synthesis products are listed as having been isolated as a residue. It will be understood by one of ordinary skill in the art that the term “residue” does not limit the physical state in which the product was isolated and may include, for example, a solid, an oil, a foam, a gum, a syrup, and the like.

### Example 1

### 4-(4,4-Dimethyl-5-oxo-3-(4-(piperidin-4-yloxy)phenyl)-2-thioxoimidazolidin-1-yl)-2-(trifluoromethyl)benzonitrile (Compound 1)

### Step A: tert-Butyl 4-(4-nitrophenoxy)piperidine-1-carboxylate, 1a

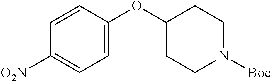 [SMILES: CC(C)(C)OC(=O)N1CCC(Oc2ccc([N+](=O)[O-])cc2)CC1]

To a solution of 1-fluoro-4-nitrobenzene (5.26 g, 37.26 mmol) in THF (135 mL) at room temperature under nitrogen, was added 1-Boc-4-hydroxypiperidine (5.0 g, 24.84 mmol). Potassium t-butoxide (5.58 g, 49.7 mmol) was added portionwise and the mixture was stirred at room temperature for 5 min. The crude mixture was poured onto water and extracted with EtOAc. The organic layer was dried over MgSO₄, filtered, and concentrated to dryness. The residue was purified by flash chromatography over silica gel (EtOAc-heptane gradient from 5% to 30%). Pure fractions were combined, concentrated, and dried under high vacuum to give compound 1a (8.0 g, 99%). MS m/z 266.9 (M+H−tBu)⁺.

### Step B: tert-Butyl 4-(4-aminophenoxy)piperidine-1-carboxylate, 1b

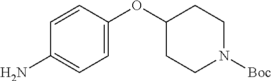 [SMILES: CC(C)(C)OC(=O)N1CCC(Oc2ccc(N)cc2)CC1]

A solution of tert-butyl 4-(4-nitrophenoxy)piperidine-1-carboxylate (8.0 g, 24.8 mmol) in MeOH (100 mL) and THF (20 mL) was purged with nitrogen, followed by the addition of Pd/C 10% wet catalyst (0.7 g) to the solution. The mixture was purged with hydrogen and stirred under a hydrogen atmosphere at room temperature for 14 h. The catalyst was removed by filtration through diatomaceous earth and the filter cake was washed with EtOAc (3×30 mL). The filtrates were evaporated under vacuum to afford the crude product. The residue was purified by flash chromatography over silica gel (EtOAc-heptane gradient from 5% to 60%). Pure fractions were combined, concentrated, and dried under high vacuum to give compound 1b (6.4 g, 88%). ¹H NMR (300 MHz, Chloroform-d) δ 1.46 (s, 9H), 1.57-1.77 (m, 2H), 1.78-1.94 (m, 2H), 3.10-3.33 (m, 2H), 3.35 (s, 2H), 3.60-3.79 (m, 2H), 4.19-4.34 (m, 1H), 6.62 (d, J=8.7 Hz, 2H), 6.75 (d, J=8.7 Hz, 2H). MS m/z 193.0 (M+H−Boc)⁺.

### Step C: tert-Butyl 4-(4-((2-cyanopropan-2-yl)amino)phenoxy)piperidine-1-carboxylate, 1c

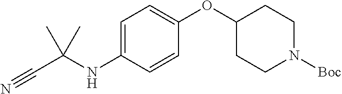 [SMILES: CC(C)(C#N)Nc1ccc(OC2CCN(C(=O)OC(C)(C)C)CC2)cc1]

Sodium cyanide (2.15 g, 43.8 mmol) was added to a solution of tert-butyl 4-(4-aminophenoxy)piperidine-1-carboxylate (6.4 g, 21.9 mmol) and acetone (3.22 mL, 43.8 mmol) in acetic acid (20 mL). The mixture was stirred at room temperature for 20 h. The solution was then poured onto a dilute aqueous NaHCO₃ solution followed by extraction with dichloromethane (3×100 mL). The organic layers were separated, then dried over MgSO₄, filtered, and concentrated to give the crude product which was used without further purification (7.01 g, 89.1%). ¹H NMR (300 MHz, Chloroform-d) δ 1.46 (s, 9H), 1.62 (s, 6H), 1.65-1.80 (m, 2H), 1.80-1.96 (m, 2H), 3.25-3.36 (m, 2H), 3.40 (s, 1H), 3.61-3.81 (m, 2H), 4.29-4.48 (m, 1H), 6.84 (d, J=8.9 Hz, 2H), 6.95 (d, J=8.9 Hz, 2H) MS m/z 333.1 (M+H−tBu)⁺.

### Step D: tert-Butyl 4-(4-(3-(4-cyano-3-(trifluoromethyl)phenyl)-5,5-dimethyl-4-oxo-2-thioxoimidazolidin-1-yl)phenoxy)piperidine-1-carboxylate, 1d

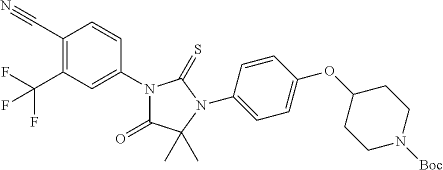 [SMILES: CC(C)(C)OC(=O)N1CCC(Oc2ccc(N3C(=S)N(c4ccc(C#N)c(C(F)(F)F)c4)C(=O)C3(C)C)cc2)CC1]

A solution of tert-butyl 4-(4-((2-cyanopropan-2-yl)amino)phenoxy)piperidine-1-carboxylate (3.50 g, 9.74 mmol) and 4-isothiocyanato-2-(trifluoromethyl)benzonitrile (2.67 g, 11.7 mmol) in DMA (50 mL) was heated to 60° C. and stirred at that temperature for 15 h. The mixture was allowed to cool to room temperature. MeOH (50 mL) and and 1M aqueous HCl (19.5 mL) were added and the mixture was stirred at room temperature for 30 min. The crude reaction mixture was quenched with saturated aqueous NaHCO₃ solution and extracted with EtOAc. The organic layer was separated and washed with brine, then dried over MgSO₄, filtered, and concentrated. The product was purified by flash chromatography over silica gel (EtOAc-heptane gradient from 5% to 50%). Product fractions were combined and concentrated to dryness to give the product (3.4 g, 59%). ¹H NMR (300 MHz, Chloroform-d) δ 1.48 (s, 9H), 1.58 (s, 6H), 1.70-1.87 (m, 2H), 1.89-2.02 (m, 2H), 3.30-3.46 (m, 2H), 3.63-3.81 (m, 2H), 4.45-4.57 (m, 1H), 7.02 (d, J=8.9 Hz, 2H), 7.20 (d, J=8.8 Hz, 2H), 7.80-7.87 (m, 1H), 7.94-8.02 (m, 2H). MS m/z 532.9 (M+H−tBu)⁺.

### Step E: 4-(4,4-Dimethyl-5-oxo-3-(4-(piperidin-4-yloxy)phenyl)-2-thioxoimidazolidin-1-yl)-2-(trifluoromethyl)benzonitrile hydrochloride, Compound 1

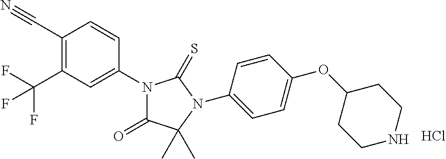 [SMILES: CC1(C)C(=O)N(c2ccc(C#N)c(C(F)(F)F)c2)C(=S)N1c1ccc(OC2CCNCC2)cc1 ; Cl]

To a solution of tert-butyl 4-(4-(3-(4-cyano-3-(trifluoromethyl)phenyl)-5,5-dimethyl-4-oxo-2-thioxoimidazolidin-1-yl)phenoxy)piperidine-1-carboxylate (3.4 g, 5.77 mmol) in dichloromethane (50 mL) was added 4N HCl solution in dioxane (14.5 mL) at 0° C. under a nitrogen atmosphere. The mixture was stirred at room temperature for 2 h, then evaporated to dryness. The solid residue was crushed and triturated with a mixture of EtOAc and Et₂O. The resulting white solid was filtered, washed with ether and heptane, and dried under high vacuum to constant weight to give the product (2.86 g, 94%). ¹H NMR (300 MHz, DMSO-d₆) δ 1.49 (s, 6H), 1.80-1.96 (m, 2H), 2.07-2.24 (m, 2H), 3.00-3.17 (m, 2H), 3.19-3.32 (m, 2H), 4.54-4.87 (m, 1H), 7.16 (d, J=9.0 Hz, 2H), 7.29 (d, J=8.9 Hz, 2H), 8.07 (dd, J=8.2, 1.9 Hz, 1H), 8.29 (d, J=1.9 Hz, 1H), 8.39 (d, J=8.3 Hz, 1H), 9.07 (s, 2H). MS m/z 488.9 (M+H)⁺.

### Example 2

### 2-Chloro-4-(4,4-dimethyl-5-oxo-3-(4-(piperidin-4-yloxy)phenyl)-2-thioxoimidazolidin-1-yl)benzonitrile hydrochloride (Compound 7)

### Step A: tert-Butyl 4-(4-(3-(3-chloro-4-cyanophenyl)-5,5-dimethyl-4-oxo-2-thioxoimid-azolidin-1-yl)phenoxy)piperidine-1-carboxylate, 2a

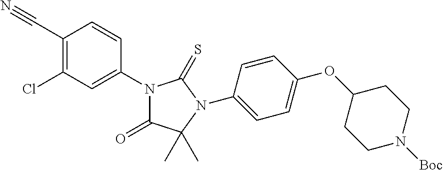 [SMILES: CC(C)(C)OC(=O)N1CCC(Oc2ccc(N3C(=S)N(c4ccc(C#N)c(Cl)c4)C(=O)C3(C)C)cc2)CC1]

Compound 2a (2.7 g, 50%) was prepared using the procedure of Example 1, STEP D, substituting 2-chloro-4-(isothiocyanato)benzonitrile for 4-isothiocyanato-2-(trifluoromethyl)benzonitrile. ¹H NMR (300 MHz, Chloroform-d) δ 1.48 (s, 9H), 1.56 (s, 6H), 1.69-1.89 (m, 2H), 1.86-2.03 (m, 2H), 3.27-3.47 (m, 2H), 3.61-3.82 (m, 2H), 4.40-4.59 (m, 1H), 7.02 (d, J=8.9 Hz, 2H), 7.19 (d, J=8.9 Hz, 2H), 7.52 (dd, J=8.4, 1.9 Hz, 1H), 7.68 (d, J=1.9 Hz, 1H), 7.80 (d, J=8.4 Hz, 1H). MS m/z 498.9 (M+H−tBu)⁺

### Step B: 2-Chloro-4-(4,4-dimethyl-5-oxo-3-(4-(piperidin-4-yloxy)phenyl)-2-thioxoimid-azolidin-1-yl)benzonitrile hydrochloride, compound 2b

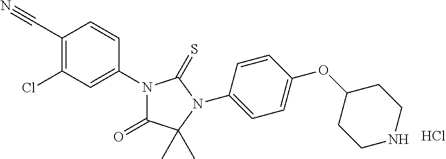 [SMILES: CC1(C)C(=O)N(c2ccc(C#N)c(Cl)c2)C(=S)N1c1ccc(OC2CCNCC2)cc1 ; Cl]

Compound 2b (2.3 g, 93%) was prepared using the procedure of Example 1, STEP E, substituting tert-butyl 4-(4-(3-(3-chloro-4-cyanophenyl)-5,5-dimethyl-4-oxo-2-thioxoimidazolidin-1-yl)phenoxy)piperidine-1-carboxylate for tert-butyl 4-(4-(3-(4-cyano-3-(trifluoromethyl)phenyl)-5,5-dimethyl-4-oxo-2-thioxoimidazolidin-1-yl)phenoxy)piperidine-1-carboxylate; m.p.>300° C. ¹H NMR (300 MHz, DMSO-d₆) δ 1.47 (s, 6H), 1.75-1.97 (m, 2H), 2.07-2.23 (m, 2H), 2.95-3.18 (m, 2H), 3.17-3.35 (m, 2H), 4.59-4.83 (m, 1H), 7.15 (d, J=8.9 Hz, 2H), 7.29 (d, J=8.8 Hz, 2H), 7.71 (dd, J=8.3, 1.9 Hz, 1H), 8.02 (d, J=1.8 Hz, 1H), 8.18 (d, J=8.3 Hz, 1H), 8.90 (s, 2H). MS m/z 455.0 (M+H)⁺.

### Example 3

### 4-(4,4-Dimethyl-3-(4-((1-methylpiperidin-4-yl)oxy)phenyl)-5-oxo-2-thioxoimidazolidin-1-yl)-2-(trifluoromethyl)benzonitrile hydrochloride (Compound 2)

### Step A: 4-(4,4-Dimethyl-5-oxo-3-(4-(piperidin-4-yloxy)phenyl)-2-thioxoimidazolidin-1-yl)-2-(trifluoromethyl)benzonitrile, Compound 2

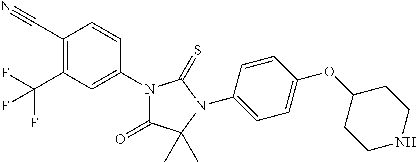 [SMILES: CC1(C)C(=O)N(c2ccc(C#N)c(C(F)(F)F)c2)C(=S)N1c1ccc(OC2CCNCC2)cc1]

4-(4,4-Dimethyl-5-oxo-3-(4-(piperidin-4-yloxy)phenyl)-2-thioxoimidazolidin-1-yl)-2-(trifluoromethyl)benzonitrile hydrochloride (2.3 g) was dissolved in dichloromethane and washed with saturated aqueous NaHCO₃ solution. The organic layer was dried over MgSO₄, filtered, and concentrated to give the product (2.13 g). MS m/z 489.0 (M+H)⁺.

### Step B: 4-(4,4-Dimethyl-5-oxo-3-(4-(1-methylpiperidin-4-yloxy)phenyl)-2-thioxo-imidazolidin-1-yl)-2-(trifluoromethyl)benzonitrile, compound 2

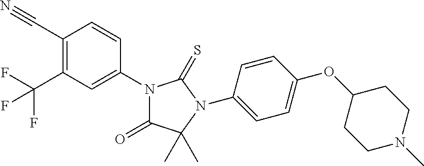 [SMILES: CN1CCC(Oc2ccc(N3C(=S)N(c4ccc(C#N)c(C(F)(F)F)c4)C(=O)C3(C)C)cc2)CC1]

Formaldehyde (37% wt in water, 0.343 mL, 4.61 mmol) was added to a solution of 4-(4,4-dimethyl-5-oxo-3-(4-(piperidin-4-yloxy)phenyl)-2-thioxoimidazolidin-1-yl)-2-(trifluoromethyl)benzonitrile (0.750 g, 1.535 mmol) in DCE (15 mL). The mixture was stirred at room temperature for 10 min, then sodium triacetoxyborohydride (0.976 g, 4.61 mmol) was added. The reaction was stirred for 15 h and was diluted with dichloromethane. The solution was washed successively with saturated aqueous NaHCO₃ solution, water, and brine. The organic layer was dried over MgSO₄, filtered and concentrated to give the crude product, which was purified by flash chromatography over silica gel (MeOH-dichloromethane gradient from 0 to 10%). Pure product fractions were combined and concentrated to dryness. The white solid was then dried under vacuum to constant weight to give the product (0.720 g, 93%). ¹H NMR (300 MHz, Chloroform-d) δ 1.58 (s, 6H), 1.78-2.25 (m, 4H), 2.43 (d, J=21.8 Hz, 5H), 2.62-2.92 (m, 2H), 4.13-4.65 (m, 1H), 6.72-7.49 (m, 4H), 7.52-8.26 (m, 3H). MS m/z 503.1 (M+H)⁺.

### Step C: 4-(4,4-Dimethyl-5-oxo-3-(4-(1-methylpiperidin-4-yloxy)phenyl)-2-thioxo-imidazolidin-1-yl)-2-(trifluoromethyl)benzonitrile hydrochloride, Compound 2

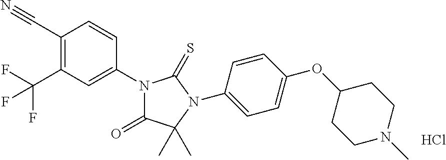 [SMILES: CN1CCC(Oc2ccc(N3C(=S)N(c4ccc(C#N)c(C(F)(F)F)c4)C(=O)C3(C)C)cc2)CC1 ; Cl]

The hydrochloride salt was prepared by addition of 4N HCl solution in dioxane to a solution of 4-(4,4-dimethyl-5-oxo-3-(4-(1-methylpiperidin-4-yloxy)phenyl)-2-thioxo-imidazolidin-1-yl)-2-(trifluoromethyl)benzonitrile in dichloromethane followed by evaporation of solvents. The white solid was then dried under vacuum to constant weight to give the product (0.495 g). ¹H NMR (300 MHz, DMSO-d₆) δ 1.49 (s, 6H), 1.85-2.34 (m, 2H), 2.76 (s, 3H), 2.98-3.24 (m, 1H), 3.43-3.81 (m, 4H), 3.94-4.17 (m, 1H), 4.50-4.91 (m, 1H), 7.17 (d, J=8.4 Hz, 2H), 7.30 (d, J=8.4 Hz, 2H), 8.08 (dd, J=8.2, 1.9 Hz, 1H), 8.29 (d, J=1.9 Hz, 1H), 8.39 (d, J=8.3 Hz, 1H), 10.87 (s, 1H). MS m/z 503.0 (M+H)⁺.

### Example 4

### 2-Chloro-4-(4,4-dimethyl-3-(4-((1-methylpiperidin-4-yl)oxy)phenyl)-5-oxo-2-thioxoimidazolidin-1-yl)benzonitrile hydrochloride (Compound 8)

### Step A: 2-Chloro-4-(4,4-dimethyl-3-(4-((1-piperidin-4-yl)oxy)phenyl)-5-oxo-2-thioxoimidazolidin-1-yl)benzonitrile, 4a

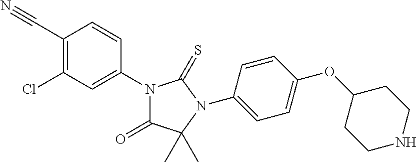 [SMILES: CC1(C)C(=O)N(c2ccc(C#N)c(Cl)c2)C(=S)N1c1ccc(OC2CCNCC2)cc1]

Compound 4a (1.7 g, 80%) was prepared using the procedure of Example 3, STEP A, substituting 2-chloro-4-(4,4-dimethyl-5-oxo-3-(4-(piperidin-4-yloxy)phenyl)-2-thioxoimidazolidin-1-yl)benzonitrile hydrochloride (Example 2, STEP B) for 4-(4,4-dimethyl-5-oxo-3-(4-(piperidin-4-yloxy)phenyl)-2-thioxoimidazolidin-1-yl)-2-(trifluoromethyl)benzonitrile hydrochloride. MS m/z 469.0 (M+H)⁺.

### Step B: 2-Chloro-4-(4,4-dimethyl-3-(4-((1-methylpiperidin-4-yl)oxy)phenyl)-5-oxo-2-thioxoimidazolidin-1-yl)benzonitrile, Compound 8

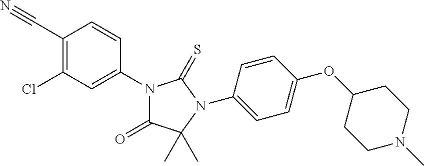 [SMILES: CN1CCC(Oc2ccc(N3C(=S)N(c4ccc(C#N)c(Cl)c4)C(=O)C3(C)C)cc2)CC1]

Compound 8 (0.987 g, 92%) was prepared using the procedure of Example 3, STEP B, substituting 2-chloro-4-(4,4-dimethyl-5-oxo-3-(4-(piperidin-4-yloxy)phenyl)-2-thioxoimidazolidin-1-yl)benzonitrile for 4-(4,4-dimethyl-5-oxo-3-(4-(piperidin-4-yloxy)phenyl)-2-thioxoimidazolidin-1-yl)-2-(trifluoromethyl)benzonitrile. m.p. 174.8° C. ¹H NMR (300 MHz, Chloroform-d) δ 1.55 (s, 6H), 1.83-2.17 (m, 4H), 2.25-2.44 (m, 5H), 2.64-2.83 (m, 2H), 4.06-4.63 (m, 1H), 6.98-7.23 (m, 4H), 7.43-7.91 (m, 3H) MS m/z 454.9 (M+H)⁺.

### Step C: 2-Chloro-4-(4,4-dimethyl-3-(4-((1-methylpiperidin-4-yl)oxy)phenyl)-5-oxo-2-thioxoimidazolidin-1-yl)benzonitrile hydrochloride, Compound 8

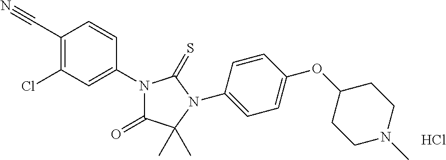 [SMILES: CN1CCC(Oc2ccc(N3C(=S)N(c4ccc(C#N)c(Cl)c4)C(=O)C3(C)C)cc2)CC1 ; Cl]

The product (0.79 g) was prepared using the procedure of Example 3, STEP C, substituting 2-chloro-4-(4,4-dimethyl-3-(4-((1-methylpiperidin-4-yl)oxy)phenyl)-5-oxo-2-thioxoimidazolidin-1-yl)benzonitrile for 4-(4,4-dimethyl-5-oxo-3-(4-(piperidin-4-yloxy)phenyl)-2-thioxoimidazolidin-1-yl)-2-(trifluoromethyl)benzonitrile. m.p. 251.7° C. ¹H NMR (300 MHz, DMSO-d₆) δ 1.47 (s, 6H), 1.82-2.00 (m, 1H), 2.02-2.21 (m, 2H), 2.21-2.35 (m, 1H), 2.78 (d, J=6.0 Hz, 3H), 3.03-3.26 (m, 2H), 3.27-3.39 (m, 1H), 3.41-3.56 (m, 1H), 4.51-4.90 (m, 1H), 7.11-7.21 (m, 2H), 7.23-7.33 (m, 2H), 7.70 (dd, J=8.3, 1.9 Hz, 1H), 8.00 (d, J=1.9 Hz, 1H), 8.15 (d, J=8.3 Hz, 1H), 10.49 (s, 1H). MS m/z 469.0 (M+H)⁺.

### Example 5

### 4-(4,4-Dimethyl-5-oxo-3-(6-(piperidin-4-yloxy)pyridin-3-yl)-2-thioxoimidazolidin-1-yl)-2-(trifluoromethyl)benzonitrile hydrochloride (Compound 3)

### Step A: tert-Butyl 4-((5-nitropyridin-2-yl)oxy)piperidine-1-carboxylate, 5a

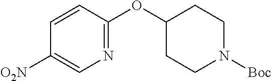 [SMILES: CC(C)(C)OC(=O)N1CCC(Oc2ccc([N+](=O)[O-])cn2)CC1]

1-Boc-4-Hydroxypiperidine (18.67 g, 90 mmol) and triphenylphosphine (54.5 g, 208 mmol) were added to a solution of 2-hydroxy-5-nitropyridine (10 g, 69.24 mmol) in THF (350 mL) at room temperature under a nitrogen atmosphere. Diisopropyl azodiacaboxylate (40.9 mL, 207.7 mmol) was added dropwise and the mixture was stirred at room temperature overnight. The crude mixture was poured onto aqueous NaHCO₃ solution and was extracted with EtOAc. The organic layer was dried over MgSO₄, filtered, and concentrated to dryness. The residue was purified by flash chromatographed over silica gel (EtOAc-heptane gradient from 5% to 30%). Pure fractions were combined, concentrated and dried under high vacuum to give the product (22.3 g, 99%). MS m/z 224.2 (M+H−Boc)⁺.

### Step B: tert-Butyl 4-((5-aminopyridin-2-yl)oxy)piperidine-1-carboxylate, 5b

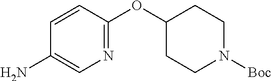 [SMILES: CC(C)(C)OC(=O)N1CCC(Oc2ccc(N)cn2)CC1]

A solution of tert-butyl 4-((5-nitropyridin-2-yl)oxy)piperidine-1-carboxylate (22.4 g, 69.24 mmol) in MeOH (210 mL) was purged with nitrogen. Then Pd/C 10% wet catalyst (1.34 g) was added to the solution. The mixture was purged with hydrogen and stirred under a hydrogen atmosphere at room temperature for 14 h. The catalyst was removed by filtration through diatomaceous earth and the filtrate was evaporated under reduced pressure to afford the product (20.3 g, 100%). ¹H NMR (300 MHz, Chloroform-d) δ 1.46 (s, 9H), 1.56-1.80 (m, 2H), 1.80-2.11 (m, 2H), 3.06-3.39 (m, 2H), 3.62-3.95 (m, 2H), 5.05 (tt, J=7.8, 3.7 Hz, 1H), 6.53-6.59 (m, 1H), 7.01 (dd, J=8.7, 3.0 Hz, 1H), 7.62 (d, J=2.9 Hz, 1H). MS m/z 294.2 (M+H)⁺.

### Step C: tert-Butyl 4-((5-((2-cyanopropan-2-yl)amino)pyridin-2-yl)oxy)piperidine-1-carboxylate, 5c

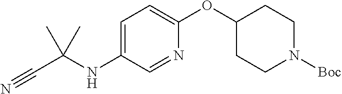 [SMILES: CC(C)(C#N)Nc1ccc(OC2CCN(C(=O)OC(C)(C)C)CC2)nc1]

Zinc cyanide (1.25 g, 10.63 mmol) was added to a solution of tert-butyl 4-((5-aminopyridin-2-yl)oxy)piperidine-1-carboxylate (2.4 g, 8.18 mmol) and acetone (0.721 mL, 9.82 mmol) in acetic acid (10 mL). The mixture was stirred at room temperature for 20 h. Additional zinc cyanide (1.25 g, 10.63 mmol) was added and the mixture was stirred for 24 h at room temperature. The solution was then poured onto a mixture of ammonia and aqueous NaHCO₃ solution followed by extraction with dichloromethane. The organic layer was separated, then dried over MgSO₄, filtered, and concentrated. The product was purified by flash chromatography over silica gel (EtAOc-heptane from 5% to 100%). Product fractions were combined and concentrated to dryness to afford the product as a beige solid (5.27 g, 64.4%). MS m/z 361.0 (M+H)⁺.

### Step D: tert-Butyl 4-((5-(3-(4-cyano-3-(trifluoromethyl)phenyl)-5,5-dimethyl-4-oxo-2-thioxoimidazolidin-1-yl)pyridin-2-yl)oxy)piperidine-1-carboxylate, 5d

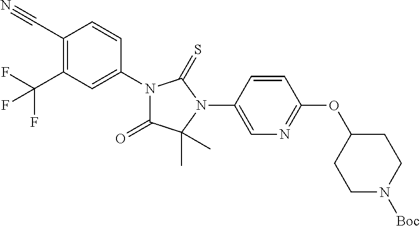 [SMILES: CC(C)(C)OC(=O)N1CCC(Oc2ccc(N3C(=S)N(c4ccc(C#N)c(C(F)(F)F)c4)C(=O)C3(C)C)cn2)CC1]

A solution of tert-butyl 4-((5-((2-cyanopropan-2-yl)amino)pyridin-2-yl)oxy)piperidine-1-carboxylate (0.940 g, 1.77 mmol) and 4-isothiocyanato-2-trifluoromethyl-benzonitrile (0.445 g, 1.05 mmol) in DMA (8 mL) was heated to 60° C. and stirred at that temperature for 15 h. The mixture was allowed to cool to room temperature. MeOH (8 mL) and 1M aqueous HCl (3.5 mL) were added and the mixture was stirred at room temperature for 30 min. The crude reaction mixture was quenched with saturated aqueous NaHCO₃ solution and was extracted with EtOAc. The organic layer was separated and washed with brine, dried over MgSO₄, filtered and concentrated. The crude product was purified by flash chromatography over silica gel (EtOAc-heptane gradient from 5% to 40%). Product fractions were combined and concentrated to dryness to give the product as a foam (0.850 g, 81%). ¹H NMR (300 MHz, Chloroform-d) δ 1.43 (s, 9H), 1.54 (s, 6H), 1.72 (dd, J=8.6, 4.3 Hz, 2H), 1.95 (dd, J=8.9, 4.9 Hz, 2H), 3.15-3.38 (m, 2H), 3.66-3.87 (m, 2H), 5.13-5.35 (m, 1H), 6.82 (d, J=8.8 Hz, 1H), 7.48 (dd, J=8.8, 2.6 Hz, 1H), 7.77-7.85 (m, 1H), 7.89-7.97 (m, 2H), 8.03 (d, J=2.6 Hz, 1H). MS m/z 533.9 (M+H−tBu)⁺.

### Step E: 4-(4,4-Dimethyl-5-oxo-3-(6-(piperidin-4-yloxy)pyridin-3-yl)-2-thioxoimidazol-idin-1-yl)-2-(trifluoromethyl)benzonitrile hydrochloride, Compound 3

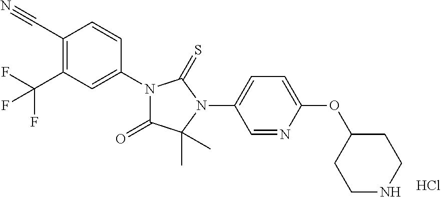 [SMILES: CC1(C)C(=O)N(c2ccc(C#N)c(C(F)(F)F)c2)C(=S)N1c1ccc(OC2CCNCC2)nc1 ; Cl]

4N HCl solution in dioxane (3.6 mL) was added to a solution of tert-butyl 4-((5-(3-(4-cyano-3-(trifluoromethyl)phenyl)-5,5-dimethyl-4-oxo-2-thioxoimidazolidin-1-yl)-pyridin-2-yl)oxy)piperidine-1-carboxylate (0.850 g, 2.15 mmol) in dichloromethane (12 mL) at 0° C. under a nitrogen atmosphere. The mixture was stirred at room temperature for 2 h, then evaporated to dryness. The residue was crushed and triturated with a mixture of EtOAc and Et₂O. The resulting white solid was collected by filtration, washed with Et₂O and heptane, and dried under high vacuum to constant weight to give the product (0.650 g, 86%). ¹H NMR (300 MHz, DMSO-d₆) δ 1.52 (s, 6H), 1.83-2.04 (m, 2H), 2.07-2.32 (m, 2H), 3.12 (d, 2H), 3.21-3.32 (m, 2H), 5.18-5.39 (m, 1H), 7.04 (d, J=8.8 Hz, 1H), 7.76 (dd, J=8.8, 2.6 Hz, 1H), 8.08 (d, J=8.0 Hz, 1H), 8.17 (d, J=2.5 Hz, 1H), 8.29 (s, 1H), 8.40 (d, J=8.3 Hz, 1H), 8.91 (s, 2H). MS m/z 490.0 (M+H)⁺.

### Example 6

### 2-Chloro-4-(4,4-dimethyl-5-oxo-3-(6-(piperidin-4-yloxy)pyridin-3-yl)-2-thioxoimidazolidin-1-yl)benzonitrile hydrochloride (Compound 9)

### Step A: tert-Butyl 4-((5-(3-(3-chloro-4-cyanophenyl)-5,5-dimethyl-4-oxo-2-thioxoimidazolidin-1-yl)pyridin-2-yl)oxy)piperidine-1-carboxylate, 6a

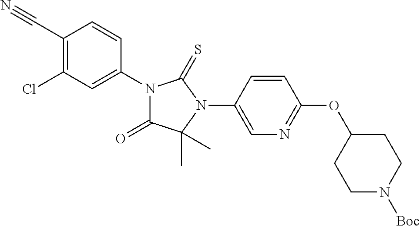 [SMILES: CC(C)(C)OC(=O)N1CCC(Oc2ccc(N3C(=S)N(c4ccc(C#N)c(Cl)c4)C(=O)C3(C)C)cn2)CC1]

Compound 6a (0.65 g, 66%) was prepared according to the procedure of Example 5, STEP D, substituting 2-chloro-4-(isothiocyanato)benzonitrile for 4-isothiocyanato-2-(trifluoromethyl)benzonitrile. ¹H NMR (300 MHz, Chloroform-d) δ 1.46 (s, 9H), 1.55 (s, 6H), 1.69-1.84 (m, 2H), 1.93-2.10 (m, 2H), 3.20-3.39 (m, 2H), 3.65-3.88 (m, 2H), 5.10-5.37 (m, 1H), 6.84 (d, J=8.8 Hz, 1H), 7.44-7.54 (m, 2H), 7.67 (d, J=1.9 Hz, 1H), 7.78 (d, J=8.3 Hz, 1H), 8.04 (d, J=2.6 Hz, 1H). MS m/z 499.9 (M+H−tBu)⁺.

### Step B: 2-Chloro-4-(4,4-dimethyl-5-oxo-3-(6-(piperidin-4-yloxy)pyridin-3-yl)-2-thioxoimidazolidin-1-yl)benzonitrile hydrochloride, Compound 9

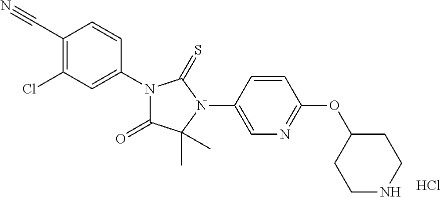 [SMILES: CC1(C)C(=O)N(c2ccc(C#N)c(Cl)c2)C(=S)N1c1ccc(OC2CCNCC2)nc1 ; Cl]

The product (0.51 g, 88%) was prepared using the procedure of Example 5, STEP E, substituting tert-butyl 4-((5-(3-(3-chloro-4-cyanophenyl)-5,5-dimethyl-4-oxo-2-thioxoimidazolidin-1-yl)pyridin-2-yl)oxy)piperidine-1-carboxylate for tert-butyl 4-((5-(3-(4-cyano-3-(trifluoromethyl)phenyl)-5,5-dimethyl-4-oxo-2-thioxoimidazolidin-1-yl)pyridin-2-yl)oxy)piperidine-1-carboxylate. m.p. 216.5° C. ¹H NMR (300 MHz, DMSO-d₆) δ 1.50 (s, 6H), 1.79-2.02 (m, 2H), 2.05-2.29 (m, 2H), 3.02-3.20 (m, 2H), 3.34 (s, 2H), 5.16-5.40 (m, 1H), 7.03 (d, J=8.8 Hz, 1H), 7.63-7.82 (m, 2H), 8.02 (d, J=1.9 Hz, 1H), 8.11-8.25 (m, 2H), 8.81 (s, 2H). MS m/z 456.0 (M+H)⁺.

### Example 7

### 4-(4,4-Dimethyl-3-(6-((1-methylpiperidin-4-yl)oxy)pyridin-3-yl)-5-oxo-2-thioxoimidazolidin-1-yl)-2-(trifluoromethyl)benzonitrile hydrochloride (Compound 4)

### Step A: 4-(4,4-Dimethyl-5-oxo-3-(6-(piperidin-4-yloxy)pyridin-3-yl)-2-thioxoimidazol-idin-1-yl)-2-(trifluoromethyl)benzonitrile, 7a

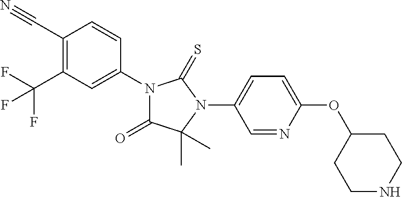 [SMILES: CC1(C)C(=O)N(c2ccc(C#N)c(C(F)(F)F)c2)C(=S)N1c1ccc(OC2CCNCC2)nc1]

4-(4,4-Dimethyl-5-oxo-3-(6-(piperidin-4-yloxy)pyridin-3-yl)-2-thioxoimidazolidin-1-yl)-2-(trifluoromethyl)benzonitrile hydrochloride (0.35 g) was dissolved in dichloromethane and washed with saturated aqueous NaHCO₃ solution. The organic layer was dried over MgSO₄, filtered, and concentrated to give compound 7a (250 mg). MS m/z 489.0 (M+H)⁺.

### Step B: 4-(4,4-Dimethyl-3-(6-((1-methylpiperidin-4-yl)oxy)pyridin-3-yl)-5-oxo-2-thioxoimidazolidin-1-yl)-2-(trifluoromethyl)benzonitrile hydrochloride, compound 4

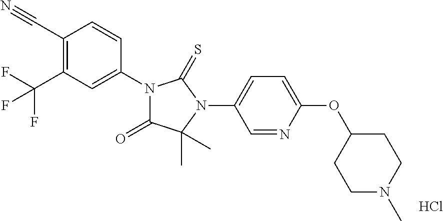 [SMILES: CN1CCC(Oc2ccc(N3C(=S)N(c4ccc(C#N)c(C(F)(F)F)c4)C(=O)C3(C)C)cn2)CC1 ; Cl]

Formaldehyde (37% wt in water, 0.114 ml, 1.53 mmol) was added to a solution of 4-(4,4-dimethyl-5-oxo-3-(6-(piperidin-4-yloxy)pyridin-3-yl)-2-thioxoimidazolidin-1-yl)-2-(trifluoromethyl)benzonitrile (0.250 g, 0.511 mmol) in DCE (15 mL). The mixture was stirred at room temperature for 10 min, then sodium triacetoxyborohydride (0.325 g, 1.53 mmol) was added. The reaction was stirred for 15 h and was diluted with dichloromethane. The solution was washed successively with saturated aqueous NaHCO₃ solution, water, and brine. The organic layer was dried over MgSO₄, filtered and concentrated to give the crude product, which was purified by flash chromatography over silica gel (MeOH-dichloromethane gradient from 0 to 10%). Pure product fractions were combined and concentrated to dryness. The hydrochloride salt was prepared by addition of 4N HCl solution in dioxane to a solution of product in dichloromethane followed by evaporation of solvents. The white solid was then dried under reduced pressure to constant weight to give the product (0.152 g, 55%). ¹H NMR (300 MHz, DMSO-d₆) δ 1.52 (s, 6H), 1.87-2.08 (m, 1H), 2.09-2.23 (m, 2H), 2.24-2.37 (m, 1H), 2.77 (dd, J=11.1, 4.6 Hz, 3H), 3.05-3.36 (m, 2H), 3.43-3.79 (m, 2H), 5.09-5.42 (m, 1H), 7.03 (dd, J=8.8, 4.6 Hz, 1H), 7.77 (q, J=4.7, 2.7 Hz, 1H), 8.08 (d, J=8.4 Hz, 1H), 8.14-8.21 (m, 1H), 8.29 (s, 1H), 8.40 (d, J=8.3 Hz, 1H), 10.22-10.85 (m, 1H). MS m/z 504.0 (M+H)⁺.

### Example 8

### 2-Chloro-4-(4,4-dimethyl-3-(6-((1-methylpiperidin-4-yl)oxy)pyridin-3-yl)-5-oxo-2-thioxoimidazolidin-1-yl)benzonitrile hydrochloride (Compound 10)

### Step A: 2-Chloro-4-(4,4-dimethyl-5-oxo-3-(6-(piperidin-4-yloxy)pyridin-3-yl)-2-thioxoimidazolidin-1-yl)benzonitrile, 8a

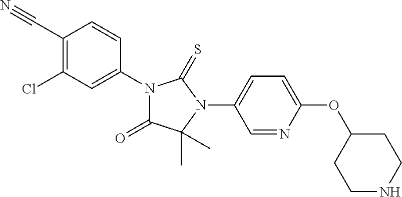 [SMILES: CC1(C)C(=O)N(c2ccc(C#N)c(Cl)c2)C(=S)N1c1ccc(OC2CCNCC2)nc1]

The product (0.290 g) was prepared using the procedure of Example 7, STEP A, substituting 2-chloro-4-(4,4-dimethyl-5-oxo-3-(6-(piperidin-4-yloxy)pyridin-3-yl)-2-thioxoimidazolidin-1-yl)benzonitrile hydrochloride (Example 6, STEP B) for 4-(4,4-dimethyl-5-oxo-3-(6-(piperidin-4-yloxy)pyridin-3-yl)-2-thioxoimidazolidin-1-yl)-2-(trifluoromethyl)benzonitrile hydrochloride. MS m/z 456.0 (M+H)⁺.

### Step B: 2-Chloro-4-(4,4-dimethyl-3-(6-((1-methylpiperidin-4-yl)oxy)pyridin-3-yl)-5-oxo-2-thioxoimidazolidin-1-yl)benzonitrile hydrochloride, Compound 10

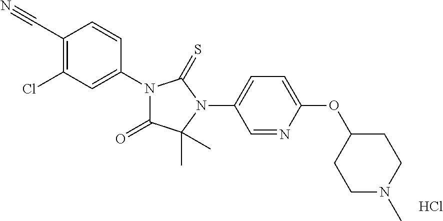 [SMILES: CN1CCC(Oc2ccc(N3C(=S)N(c4ccc(C#N)c(Cl)c4)C(=O)C3(C)C)cn2)CC1 ; Cl]

Compound 10 (0.12 g, 64%) was prepared using the procedure of Example 7, STEP B, substituting 2-chloro-4-(4,4-dimethyl-5-oxo-3-(6-(piperidin-4-yloxy)pyridin-3-yl)-2-thioxoimidazolidin-1-yl)benzonitrile for 4-(4,4-dimethyl-5-oxo-3-(6-(piperidin-4-yloxy)pyridin-3-yl)-2-thioxoimidazolidin-1-yl)-2-(trifluoromethyl)benzonitrile. m.p. 220.9° C. ¹H NMR (300 MHz, DMSO-d₆) δ 1.50 (s, 6H), 1.88-2.35 (m, 4H), 2.69-2.83 (m, 3H), 3.09-3.24 (m, 2H), 3.28-3.80 (m, 2H), 5.12-5.43 (m, 1H), 6.96-7.12 (m, 1H), 7.65-7.86 (m, 2H), 8.02 (s, 1H), 8.10-8.27 (m, 2H), 10.50-10.97 (m, 1H) MS m/z 470.0 (M+H)⁺.

### Example 9

### 4-(4,4-Dimethyl-5-oxo-3-(2-(piperidin-4-yloxy)pyrimidin-5-yl)-2-thioxoimidazolidin-1-yl)-2-(trifluoromethyl)benzonitrile (Compound 5)

### Step A: tert-Butyl 4-((5-nitropyrimidin-2-yl)oxy)piperidine-1-carboxylate, 9a

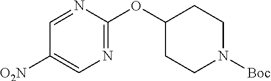 [SMILES: CC(C)(C)OC(=O)N1CCC(Oc2ncc([N+](=O)[O-])cn2)CC1]

Cesium fluoride (17.8 g, 117.5 mmol) was added to a solution of 2-chloro-5-nitro-pyrimidine (12.5 g, 78.3 mmol) and tert-butyl 4-hydroxypiperidine-1-carboxylate (15.8 g, 78.3 mmol) in DMF (375 mL). The resulting mixture was stirred for 24 h at room temperature. The insoluble solids were removed by filtration were filtered through a short pad of diatomaceous earth and the filtrate was concentrated under reduced pressure. The residue was dissolved in EtOAc (200 mL) and washed successively with water (150 mL) and brine (50 mL). The organic layer was dried over MgSO₄, filtered and concentrated to dryness. Purification by flash chromatography over silica gel (gradient of EtOAc in heptane from 0 to 35%) gave the pure compound 9a as a white solid (13.9 g, 54%). ¹H NMR (300 MHz, Chloroform-d) δ 1.47 (s, 9H), 1.76-1.92 (m, 2H), 1.98-2.13 (m, 2H), 3.21-3.44 (m, 2H), 3.63-4.00 (m, 2H), 5.26-5.50 (m, 1H), 9.29 (s, 2H). MS m/z 269 (M+H−tBu)⁺.

### Step B: tert-Butyl 4-((5-aminopyrimidin-2-yl)oxy)piperidine-1-carboxylate, 9b

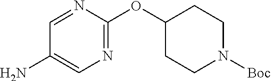 [SMILES: CC(C)(C)OC(=O)N1CCC(Oc2ncc(N)cn2)CC1]

tert-Butyl 4-((5-nitropyrimidin-2-yl)oxy)piperidine-1-carboxylate (13.9 g, 43.0 mmol) was dissolved in MeOH (200 mL) and cooled in ice/water bath under a nitrogen stream. Dry 10% Pd/C (2.79 g) was added to the cold solution and the reaction vessel was connected to a balloon filled with hydrogen gas. The suspension was then stirred under a hydrogen atmosphere at room temperature for 3 h. The catalyst was removed by filtration through a pad of diatomaceous earth. The filtrate was concentrated to give the crude product that was used without further treatment (12.2 g, 96%). ¹H NMR (300 MHz, Chloroform-d) δ 1.46 (s, 9H), 1.69-1.86 (m, 2H), 1.89-2.08 (m, 2H), 3.20-3.34 (m, 2H), 3.38 (br s, 2H), 3.65-3.90 (m, 2H), 4.90-5.16 (m, 1H), 8.03 (s, 2H). MS m/z 295 (M+H)⁺.

### Step C: tert-Butyl 4-((5-((2-cyanopropan-2-yl)amino)pyrimidin-2-yl)oxy)piperidine-1-carboxylate, 9c

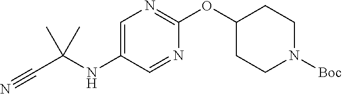 [SMILES: CC(C)(C#N)Nc1cnc(OC2CCN(C(=O)OC(C)(C)C)CC2)nc1]

Sodium cyanide (0.100 g, 2.04 mmol) was added to a solution of tert-butyl 4-((5-aminopyrimidin-2-yl)oxy)piperidine-1-carboxylate (0.3 g, 1.02 mmol) and acetone (0.15 mL, 2.04 mmol) in acetic acid (10 mL). The mixture was stirred at room temperature overnight. The solution was then poured onto 1M aqueous Na₂CO₃ solution (20 mL) followed by extraction with EtOAc (20 mL). The organic layers were separated, then dried over MgSO₄, filtered, and concentrated. The crude product was purified by column chromatography over silica gel (MeOH-dichloromethane gradient from 0% to 10%). Product fractions were combined and concentrated to dryness to give compound 9c (0.283 g). ¹H NMR (300 MHz, Chloroform-d) δ 1.47 (s, 9H), 1.58 (br s, 1H), 1.63 (s, 6H), 1.71-1.89 (m, 2H), 1.91-2.07 (m, 2H), 3.20-3.45 (m, 2H), 3.69-3.90 (m, 2H), 4.97-5.24 (m, 1H), 8.32 (s, 2H). MS m/z 362.1 (M+H)⁺.

### Step D: tert-Butyl 4-((5-(3-(4-cyano-3-(trifluoromethyl)phenyl)-5,5-dimethyl-4-oxo-2-thioxoimidazolidin-1-yl)pyrimidin-2-yl)oxy)piperidine-1-carboxylate, 9d

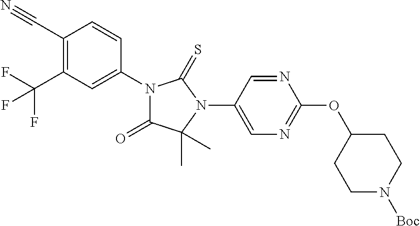 [SMILES: CC(C)(C)OC(=O)N1CCC(Oc2ncc(N3C(=S)N(c4ccc(C#N)c(C(F)(F)F)c4)C(=O)C3(C)C)cn2)CC1]

A solution of tert-butyl 4-((5-((2-cyanopropan-2-yl)amino)pyrimidin-2-yl)oxy)piperidine-1-carboxylate (0.283 g, 0.783 mmol) and 4-isothiocyanato-2-(trifluoromethyl)benzonitrile (0.268 g, 1.17 mmol) in DMA (10 mL) was heated to 60° C. and stirred at that temperature for 2 h. Additional 4-isothiocyanato-2-(trifluoromethyl)benzonitrile (0.306 g, 1.34 mmol) was added and stirring at 60° C. was continued for 2 h. MeOH (2 mL) and 1M aqueous HCl (2 mL) were added and the mixture was stirred at room temperature for 1 h. EtOAc (50 mL) was added and the solution was washed with 1M aqueous Na₂CO₃ solution (150 mL). The organic layer was separated, dried over MgSO₄, filtered, and concentrated. The product was purified by chromatography over silica gel (EtOAc-heptane gradient from 0% to 50%). Product fractions were combined and concentrated to dryness. The amorphous solid residue was triturated with Et₂O. A resultant precipitate impurity was removed by filtration and discarded, and the filtrate was concentrated to give compound 9d (0.255 g, 42%). ¹H NMR (300 MHz, Chloroform-d) δ 1.49 (s, 9H), 1.62 (s, 6H), 1.78-1.96 (m, 2H), 1.96-2.13 (m, 2H), 3.26-3.43 (m, 2H), 3.71-3.92 (m, 2H), 5.18-5.34 (m, 1H), 7.80-7.87 (m, J=9.0 Hz, 1H), 7.95 (s, 1H), 7.98 (d, J=9.0 Hz, 1H), 8.47 (s, 2H). MS m/z 534.9 (M+H−tBu).

### Step E: 4-(4,4-Dimethyl-5-oxo-3-(2-(piperidin-4-yloxy)pyrimidin-5-yl)-2-thioxo-imidazolidin-1-yl)-2-(trifluoromethyl)benzonitrile, Compound 5

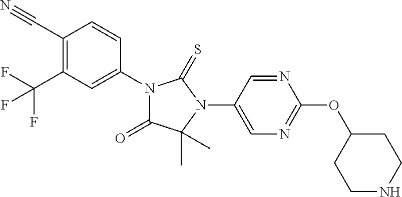 [SMILES: CC1(C)C(=O)N(c2ccc(C#N)c(C(F)(F)F)c2)C(=S)N1c1cnc(OC2CCNCC2)nc1]

TFA (1 mL) was added to a solution of tert-butyl 4-((5-(3-(4-cyano-3-(trifluoromethyl)phenyl)-5,5-dimethyl-4-oxo-2-thioxoimidazolidin-1-yl)pyrimidin-2-yl)oxy)piperidine-1-carboxylate (0.255 g, 0.432 mmol) in dichloromethane (5 mL). The mixture was stirred at room temperature for 2 h, then evaporated to dryness. The residue was dissolved in toluene (15 mL) and again concentrated (3×). The crude oily residue was filtered through a short column of silica gel (MeOH-dichloromethane gradient from 0% to 10%) to give a material that was further purified by preparative reverse phase HPLC (C₁₈, ACN-(25 mM aqueous NH₄CO₃) gradient from 25% to 38%) to give compound 5 (0.097 g, 46%). m.p. 239.0° C. ¹H NMR (300 MHz, Chloroform-d) δ 1.63 (s, 6H), 2.19-2.45 (m, 4H), 3.20-3.37 (m, 2H), 3.38-3.55 (m, 2H), 5.34-5.48 (m, 1H), 7.82 (dd, J=8.3, 2.1 Hz, 1H), 7.94 (d, J=2.0 Hz, 1H), 8.00 (d, J=8.3 Hz, 1H), 8.50 (s, 2H), 9.61 (br s, 1H). MS m/z 491.0 (M+H)⁺.

### Example 10

### 4-(4,4-Dimethyl-3-(2-((1-methylpiperidin-4-yl)oxy)pyrimidin-5-yl)-5-oxo-2-thioxoimidazolidin-1-yl)-2-(trifluoromethyl)benzonitrile (Compound 6)

### Step A: 2-((1-Methylpiperidin-4-yl)oxy)-5-nitropyrimidine, 10a

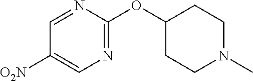 [SMILES: CN1CCC(Oc2ncc([N+](=O)[O-])cn2)CC1]

NaH (60% oil dispersion, 8 g, 333 mmol) was added to a solution of 1-methyl-4-hydroxypiperidine (18 g, 156 mmol) in dry DMF (200 mL) at 6° C. The solution was stirred at room temperature for 30 min and a solution of 2-chloro-5-nitropyrimidine (25 g, 125 mmol) in dry DMF (50 mL) was then added. The mixture was stirred at room temperature overnight. Water (50 mL) was added and the solution was extracted with EtOAc (3×100 mL). The organic layers were combined, concentrated under vacuum, and purified by column chromatography over silica gel (MeOH-dichloromethane gradient from 1% to 3.3%). The desired fractions were collected and evaporated to give compound 10a (13 g, 43%).

### Step B: 2-((1-Methylpiperidin-4-yl)oxy)pyrimidin-5-amine, 10b

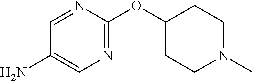

A solution of tert-butyl 4-((5-nitropyridin-2-yl)oxy)piperidine-1-carboxylate (13 g, 54.6 mmol) in MeOH (200 mL) was purged with nitrogen. Then Pd/C 10% catalyst (1.3 g) was added to the solution. The mixture was purged with hydrogen and stirred under a hydrogen atmosphere at room temperature overnight. The catalyst was removed by filtration through diatomaceous earth and the filtrate was evaporated under reduced pressure. The resulting residue was washed with isopropyl ether to give the product (9 g, 79%). MS m/z 209.1 (M+H)⁺.

### Step C: 2-Methyl-2-((2-((1-methylpiperidin-4-yl)oxy)pyrimidin-5-yl)amino)propane-nitrile, 10c

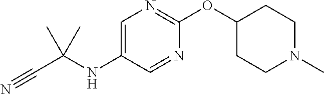 [SMILES: CN1CCC(Oc2ncc(NC(C)(C)C#N)cn2)CC1]

Sodium cyanide (0.235 g, 4.80 mmol) was added to a solution of 2-((1-methylpiperidin-4-yl)oxy)pyrimidin-5-amine (0.5 g, 2.40 mmol) and acetone (0.353 mL, 4.80 mmol) in acetic acid (10 mL). The mixture was stirred at room temperature overnight. The solution was then partitioned between 1M aqueous Na₂CO₃ solution (20 mL) and EtOAc (20 mL). The organic layers were separated, dried over MgSO₄, filtered, and concentrated. The crude product was purified by column chromatography over silica gel (1% NH₃/9% MeOH/90% dichloromethane)-dichloromethane gradient from 0% to 100%). Product fractions were combined and concentrated to dryness to give compound 10c (0.462 g, 66%). ¹H NMR (300 MHz, Chloroform-d) δ 1.62 (s, 6H), 1.66 (br s, 1H), 1.86-2.01 (m, 2H), 2.01-2.15 (m, 2H), 2.23-2.40 (m, 2H), 2.31 (s, 3H), 2.59-2.84 (m, 2H), 4.85-5.12 (m, 1H), 8.32 (s, 2H). MS m/z 276.0 (M+H)⁺.

### Step D: 4-(4,4-Dimethyl-3-(2-((1-methylpiperidin-4-yl)oxy)pyrimidin-5-yl)-5-oxo-2-thioxoimidazolidin-1-yl)-2-(trifluoromethyl)benzonitrile, Compound 6

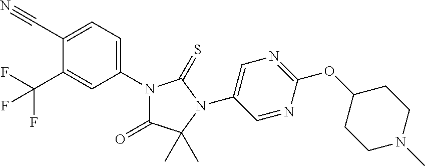 [SMILES: CN1CCC(Oc2ncc(N3C(=S)N(c4ccc(C#N)c(C(F)(F)F)c4)C(=O)C3(C)C)cn2)CC1]

A solution of 2-methyl-2-((2-((1-methylpiperidin-4-yl)oxy)pyrimidin-5-yl)amino)propanenitrile (0.467 g, 1.70 mmol) and 4-isothiocyanato-2-(trifluoromethyl)benzonitrile (0.581 g, 2.54 mmol) in DMA (10 mL) was heated to 60° C. overnight. The mixture was allowed to cool to room temperature and MeOH (2 mL) and 1M aqueous HCl (2 mL) was added. After stirring at room temperature for 1 h, EtOAc (50 mL) was added and the organic layer was washed with 1M aqueous Na₂CO₃ solution (150 mL). The organic layer was separated, dried over MgSO₄, filtered, and concentrated. The crude product was filtered through a short column of silica gel (MeOH-dichloromethane gradient from 0% to 10%). The residue was then purified by preparative reverse phase HPLC (C₁₈, ACN-(0.1% aqueous formic acid) gradient from 5% to 37%). Fractions containing product were treated with aqueous NaHCO₃ solution and extracted with EtOAc. The organic layer was concentrated and the solid residue was triturated with Et₂O to give the product (0.056 g, 6.5%); m.p. 204.9° C. ¹H NMR (300 MHz, DMSO-d₆) δ 1.55 (s, 6H), 1.67-1.87 (m, 2H), 1.95-2.12 (m, 2H), 2.12-2.30 (m, 2H), 2.20 (s, 3H), 2.59-2.73 (m, 2H), 4.90-5.10 (s, 1H), 8.07 (d, J=8.3 Hz, 1H), 8.29 (s, 1H), 8.40 (d, J=8.3 Hz, 1H), 8.64 (s, 2H). MS m/z 505.0 (M+H)⁺.

### Example 11

### 4-(8-Oxo-5-(4-(piperidin-4-yloxy)phenyl)-6-thioxo-5,7-diazaspiro[3.4]octan-7-yl)-2-(trifluoromethyl)benzonitrile (Compound 11)

### Step A: 1-((4-Hydroxyphenyl)amino)cyclobutane-1-carbonitrile, 11a

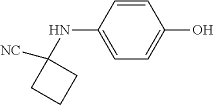 [SMILES: N#CC1(Nc2ccc(O)cc2)CCC1]

Trimethylsilanecarbonitrile (136 g, 1.37 mol) was added to a solution of 4-aminophenol (100 g, 0.916 mol) and cyclobutanone (96.3 g, 1.37 mol) and the mixture was stirred at room temperature for 24 h. The crude product was purified by column chromatography over silica gel (EtOAc-petroleum ether gradient from 10% to 50%) to give compound 11a (66 g, 37%). ¹H NMR (400 MHz, Chloroform-d) δ 2.00-2.24 (m, 2H) 2.26-2.42 (m, 2H) 2.57-2.92 (m, 2H) 3.63-3.98 (m, 1H) 5.35-5.70 (m, 1H) 6.54 (d, J=8.56 Hz, 2H) 6.71 (d, J=8.80 Hz, 2H). MS m/z 189.1 (M+H)⁺.

### Step B: 4-(5-(4-Hydroxyphenyl)-8-oxo-6-thioxo-5,7-diazaspiro[3.4]octan-7-yl)-2-(trifluoromethyl)benzonitrile, 11b

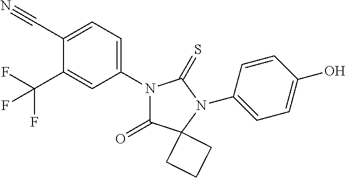 [SMILES: N#Cc1ccc(N2C(=O)C3(CCC3)N(c3ccc(O)cc3)C2=S)cc1C(F)(F)F]

A solution of 1-((4-hydroxyphenyl)amino)cyclobutane-1-carbonitrile (0.50 g, 2.66 mmol) and 4-isothiocyanato-2-trifluoromethyl-benzonitrile (0.909 g, 3.99 mmol) in DMA (10 mL) was heated to 60° C. for 2 h. The mixture was allowed to cool to room temperature. MeOH (2 mL) and 1M aqueous HCl (2 mL) were added and the mixture was stirred at room temperature for 1 h. The crude reaction mixture was diluted with EtOAc (50 mL). The organic mixture was washed with 1M aqueous Na₂CO₃ solution (150 mL) and brine, then dried over MgSO₄, filtered and concentrated. The crude product was purified by flash chromatography over silica gel (MeOH-dichloromethane gradient from 0% to 10%). Product fractions were combined and concentrated to dryness to give the product as a foam (0.963 g, 83%). ¹H NMR (300 MHz, DMSO-d₆) δ 1.43-1.62 (m, 1H), 1.84-2.04 (m, 1H), 2.30-2.47 (m, 2H), 2.53-2.67 (m, 2H), 6.94 (d, J=8.6 Hz, 2H), 7.19 (d, J=8.6 Hz, 2H), 8.05 (dd, J=8.2, 1.9 Hz, 1H), 8.25 (d, J=1.9 Hz, 1H), 8.36 (d, J=8.3 Hz, 1H), 9.90 (s, 1H). MS m/z 417.9 (M+H)⁺.

### Step C: tert-Butyl 4-(4-(7-(4-cyano-3-(trifluoromethyl)phenyl)-8-oxo-6-thioxo-5,7-diazaspiro[3.4]octan-5-yl)phenoxy)piperidine-1-carboxylate, 11c

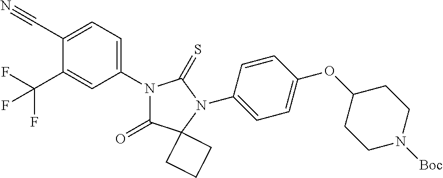 [SMILES: CC(C)(C)OC(=O)N1CCC(Oc2ccc(N3C(=S)N(c4ccc(C#N)c(C(F)(F)F)c4)C(=O)C34CCC4)cc2)CC1]

A solution 4-(5-(4-hydroxyphenyl)-8-oxo-6-thioxo-5,7-diazaspiro[3.4]octan-7-yl)-2-(trifluoromethyl)benzonitrile (0.96 g, 2.31 mmol), tert-butyl 4-hydroxypiperidine-1-carboxylate (1.02 g, 5.07 mmol), and triphenylphosphine (2.66 g, 10.1 mmol) in dry THF (20 mL) under nitrogen atmosphere was heated at 60° C. A solution of diisopropyl azodicarboxylate (1.82 mL, 9.23 mmol) in THF (10 mL) was added dropwise. Once the addition was complete, the stirring was continued overnight at the same temperature. The mixture was then allowed to cool to room temperature and diluted with EtOAc (50 mL). The solution was washed with saturated aqueous NaHCO₃ solution (15 mL) and brine (15 mL). The organic phase was dried over MgSO₄, filtered, and concentrated to dryness. The crude residue was purified by column chromatography on silica gel (EA-heptane gradient from 5% to 30%). The fractions with product were concentrated to give compound 11c as an amorphous solid (1.69 g, 97%). ¹H NMR (300 MHz, Chloroform-d) δ 1.48 (s, 9H), 1.60-1.74 (m, 1H), 1.74-1.88 (m, 2H), 1.91-2.03 (m, 2H), 2.17-2.30 (m, 1H), 2.45-2.75 (m, 4H), 3.27-3.47 (m, 2H), 3.62-3.83 (m, 2H), 4.54 (m, 1H), 7.07 (d, J=8.8 Hz, 2H), 7.22 (d, J=8.8 Hz, 2H), 7.85 (dd, J=8.3, 1.9 Hz, 1H), 7.91-8.02 (m, 2H). MS m/z 545 (M+H−tBu)⁺; MS m/z 545.0 (M+H−tBu)⁺.

### Step D: 4-(8-Oxo-5-(4-(piperidin-4-yloxy)phenyl)-6-thioxo-5,7-diazaspiro[3.4]octan-7-yl)-2-(trifluoromethyl)benzonitrile, compound 11

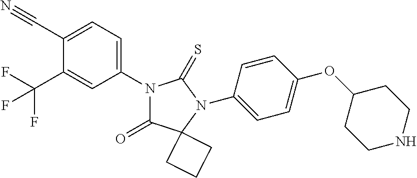 [SMILES: N#Cc1ccc(N2C(=O)C3(CCC3)N(c3ccc(OC4CCNCC4)cc3)C2=S)cc1C(F)(F)F]

TF (5 mL) was added to a solution of tert-butyl 4-(4-(7-(4-cyano-3-(trifluoromethyl)phenyl)-8-oxo-6-thioxo-5,7-diazaspiro[3.4]octan-5-yl)phenoxy)piperidine-1-carboxylate (1.69 g, 2.81 mmol) in dichloromethane (20 mL) with stirring. The mixture was stirred for 2 h at room temperature and concentrated under reduced pressure. The residue was dissolved in toluene (15 mL) and again concentrated (3×). The crude residue was then purified by column chromatography over silica gel (MeOH-dichloromethane gradient from 0% to 5%) to afford the product as a white solid (1.53 g, 71%)%). m.p. 172.4° C. ¹H NMR (300 MHz, DMSO-d₆) δ 1.43-1.62 (m, 1H), 1.78-2.03 (m, 3H), 2.06-2.24 (m, 2H), 2.33-2.47 (m, 2H), 2.54-2.70 (m, 2H), 3.01-3.21 (m, 2H), 3.24-3.33 (m, 2H), 4.56-4.82 (m, 1H), 7.20 (d, J=8.8 Hz, 2H), 7.34 (d, J=8.8 Hz, 2H), 8.05 (d, J=8.5 Hz, 1H), 8.24 (s, 1H), 8.37 (d, J=8.3 Hz, 1H), 8.53 (br s, 2H). MS m/z 501 (M+H)⁺.

### Example 12

### 2-Chloro-4-(8-oxo-5-(4-(piperidin-4-yloxy)phenyl)-6-thioxo-5,7-diazaspiro[3.4]octan-7-yl)benzonitrile (Compound 21)

### Step A: 2-Chloro-4-(5-(4-hydroxyphenyl)-8-oxo-6-thioxo-5,7-diazaspiro[3.4]octan-7-yl)benzonitrile, 12a

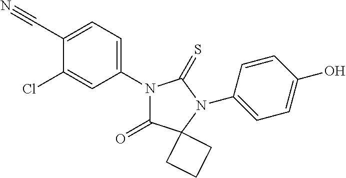 [SMILES: N#Cc1ccc(N2C(=O)C3(CCC3)N(c3ccc(O)cc3)C2=S)cc1Cl]

A solution of 1-((4-Hydroxyphenyl)amino)cyclobutane-1-carbonitrile (Example 11, STEP A) (0.50 g, 2.65 mmol) and 2-chloro-4-isothiocyanatobenzonitrile (0.776 g, 3.98 mmol) in DMA (10 mL) was heated for 2 h at 60° C. and then allowed to cool to room temperature. The mixture was diluted with MeOH (2 mL) and 1M aqueous HCl solution (2 mL) was added. The stirring was maintained at room temperature for 1 h. EtOAc (50 mL) was added and solution washed with 1M aqueous Na₂CO₃ solution (150 mL). The organic layer was dried over MgSO₄, filtered, and concentrated to dryness. The crude residue was passed through a short column of silica gel (MeOH-DCM from gradient from 0% to 10%). The fractions with product were collected and concentrated under reduced pressure to give an amorphous solid (1.28 g, 100%). ¹H NMR (300 MHz, Chloroform-d) δ 1.45-1.66 (m, 1H), 1.87-2.07 (m, 1H), 2.35-2.49 (m, 2H), 2.54-2.71 (m, 2H), 6.03 (br s, 1H), 7.03 (d, J=8.6 Hz, 2H), 7.17 (d, J=8.5 Hz, 2H), 7.53 (dd, J=8.4, 1.9 Hz, 1H), 7.69 (d, J=1.9 Hz, 1H), 7.79 (d, J=8.4 Hz, 1H). MS m/z 383.8 (M+H)⁺.

### Step B: tert-Butyl 4-(4-(7-(3-chloro-4-cyanophenyl)-8-oxo-6-thioxo-5,7-diazaspiro[3.4]octan-5-yl)phenoxy)piperidine-1-carboxylate, 12b

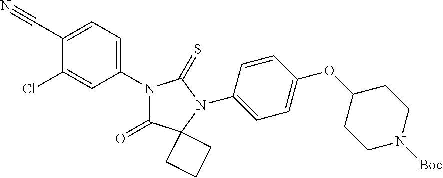 [SMILES: CC(C)(C)OC(=O)N1CCC(Oc2ccc(N3C(=S)N(c4ccc(C#N)c(Cl)c4)C(=O)C34CCC4)cc2)CC1]

2-Chloro-4-(5-(4-hydroxyphenyl)-8-oxo-6-thioxo-5,7-diazaspiro[3.4]octan-7-yl)benzonitrile (1.28 g, 3.33 mmol), tert-butyl 4-hydroxypiperidine-1-carboxylate (0.74 g, 3.67 mmol), and triphenylphosphine (1.92 g, 7.33 mmol) were dissolved in dry THF (20 mL) under nitrogen atmosphere and heated at 60° C. A solution of diisopropyl azodicarboxylate (1.31 mL, 6.67 mmol) in THF (10 mL) was added dropwise. Once the addition was complete, stirring was continued for 4 h at the same temperature. The mixture was then allowed to cool and was diluted with EtOAc (50 mL). The solution was washed with saturated aqueous NaHCO₃ solution (15 mL) and brine (15 mL). The organic phase was dried over MgSO4, filtered, and concentrated to dryness. The crude residue was filtered through a column of silica gel (EtOAc-heptane gradient from 5% to 30%). The fractions with product were concentrated to an amorphous solid (1.64 g, 86%). MS m/z 567 (M+H)⁺.

### Step C: 2-Chloro-4-(8-oxo-5-(4-(piperidin-4-yloxy)phenyl)-6-thioxo-5,7-diazaspiro[3.4]octan-7-yl)benzonitrile, compound 21

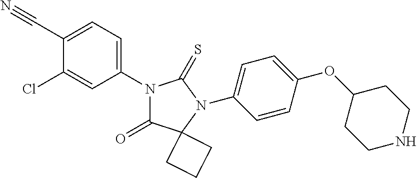 [SMILES: N#Cc1ccc(N2C(=O)C3(CCC3)N(c3ccc(OC4CCNCC4)cc3)C2=S)cc1Cl]

TFA (2 mL) was added with stirring to a solution of tert-butyl 4-(4-(7-(3-chloro-4-cyanophenyl)-8-oxo-6-thioxo-5,7-diazaspiro[3.4]octan-5-yl)phenoxy)piperidine-1-carboxylate (1.64 g, 2.88 mmol) in dichloromethane (10 mL). The mixture was stirred for 2 h at room temperature and concentrated under reduced pressure. The residue was dissolved in toluene (15 mL) and again concentrated (3×). The crude residue was then purified by column chromatography over silica gel (MeOH-dichloromethane gradient from 0% to 10%) to afford a solid (0.543 g, 39%). m.p. 211.0° C. ¹H NMR (300 MHz, Chloroform-d) δ 1.57-1.79 (m, 1H), 2.18-2.37 (m, 5H), 2.43-2.75 (m, 4H), 3.22-3.54 (m, 4H), 4.65-4.81 (m, 1H), 7.08 (d, J=8.7 Hz, 2H), 7.25 (d, J=8.7 Hz, 2H), 7.52 (dd, J=8.3, 2.0 Hz, 1H), 7.69 (d, J=1.9 Hz, 1H), 7.80 (d, J=8.4 Hz, 1H), 9.15 (br s, 2H). MS m/z 467.0 (M+H)⁺.

### Example 13

### 2-Methyl-4-(5-(4-((1-methylpiperidin-4-yl)oxy)phenyl)-8-oxo-6-thioxo-5,7-diazaspiro[3.4]octan-7-yl)benzonitrile (Compound 17)

### Step A: 4-(5-(4-Hydroxyphenyl)-8-oxo-6-thioxo-5,7-diazaspiro[3.4]octan-7-yl)-2-methylbenzonitrile, 13a

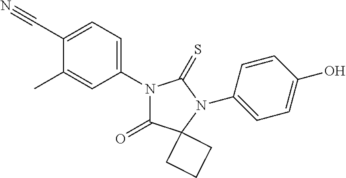 [SMILES: Cc1cc(N2C(=O)C3(CCC3)N(c3ccc(O)cc3)C2=S)ccc1C#N]

Compound 13a (0.122 g) was prepared using the procedure of Example 11, STEP B, substituting 4-isothiocyanato-2-methylbenzonitrile for 4-isothiocyanato-2-trifluoromethyl-benzonitrile. MS m/z 364 (M+H)⁺.

### Step B: 2-Methyl-4-(5-(4-((1-methylpiperidin-4-yl)oxy)phenyl)-8-oxo-6-thioxo-5,7-diazaspiro[3.4]octan-7-yl)benzonitrile, compound 17

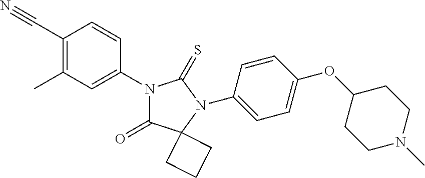 [SMILES: Cc1cc(N2C(=O)C3(CCC3)N(c3ccc(OC4CCN(C)CC4)cc3)C2=S)ccc1C#N]

Compound 17 (0.123 g, 87%) was prepared using the procedure of Example 11, STEP C, substituting 4-(5-(4-hydroxyphenyl)-8-oxo-6-thioxo-5,7-diazaspiro[3.4]octan-7-yl)-2-methylbenzonitrile for 4-(5-(4-hydroxyphenyl)-8-oxo-6-thioxo-5,7-diazaspiro[3.4]octan-7-yl)-2-(trifluoromethyl)benzonitrile and substituting 1-methyl-4-hydroxypiperidine for tert-butyl 4-hydroxypiperidine-1-carboxylate. ¹H NMR (400 MHz, Chloroform-d) δ ppm 1.55-1.75 (m, 2H), 1.93 (br s, 2H), 2.09 (br s, 2H), 2.21 (dq, J=19.3, 9.4 Hz, 2H), 2.36 (br s, 3H), 2.47-2.59 (m, 2H), 2.63 (br s, 2H), 2.61 (s, 3H), 2.75 (br s, 2H), 4.40 (br s, 1H), 7.06 (d, J=8.3 Hz, 2H), 7.22 (d, J=8.6 Hz, 2H), 7.38 (d, J=8.3 Hz, 1H), 7.42 (s, 1H), 7.74 (d, J=8.1 Hz, 1H). MS m/z 461 (M+H)⁺.

### Example 14

### 2-Methoxy-4-(5-(4-((1-methylpiperidin-4-yl)oxy)phenyl)-8-oxo-6-thioxo-5,7-diazaspiro[3.4]octan-7-yl)benzonitrile (Compound 19)

### Step A: 4-(5-(4-Hydroxyphenyl)-8-oxo-6-thioxo-5,7-diazaspiro[3.4]octan-7-yl)-2-methoxybenzonitrile, 14a

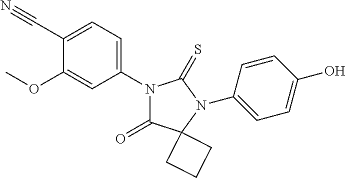 [SMILES: COc1cc(N2C(=O)C3(CCC3)N(c3ccc(O)cc3)C2=S)ccc1C#N]

Compound 14a (0.236 g) was prepared according to the procedure of Example 11, STEP B, substituting 4-isothiocyanato-2-methoxybenzonitrile for 4-isothiocyanato-2-trifluoromethyl-benzonitrile. MS m/z 380 (M+H)⁺.

### Step B: 2-Methoxy-4-(5-(4-((1-methylpiperidin-4-yl)oxy)phenyl)-8-oxo-6-thioxo-5,7-diazaspiro[3.4]octan-7-yl)benzonitrile, compound 19

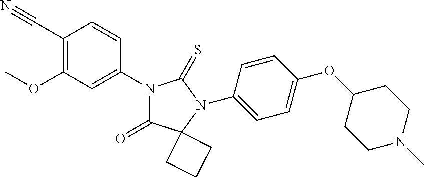 [SMILES: COc1cc(N2C(=O)C3(CCC3)N(c3ccc(OC4CCN(C)CC4)cc3)C2=S)ccc1C#N]

Compound 19 (0.078 g) was prepared using the procedure of Example 11, STEP C, substituting 4-(5-(4-hydroxyphenyl)-8-oxo-6-thioxo-5,7-diazaspiro[3.4]octan-7-yl)-2-methoxybenzonitrile for 4-(5-(4-hydroxyphenyl)-8-oxo-6-thioxo-5,7-diazaspiro[3.4]octan-7-yl)-2-(trifluoromethyl)benzonitrile and substituting 1-methyl-4-hydroxypiperidine for tert-butyl 4-hydroxypiperidine-1-carboxylate. ¹H NMR (400 MHz, Chloroform-d) δ ppm 1.53-1.73 (m, 2H), 1.95 (br s, 2H), 2.12 (br s, 2H), 2.17-2.29 (m, 1H), 2.38 (br s, 3H), 2.41-2.49 (m, 1H), 2.49-2.59 (m, 2H), 2.60-2.69 (m, 2H), 2.78 (br s, 2H), 3.97 (s, 3H), 4.43 (br s, 1H), 7.03-7.15 (m, 4H), 7.23 (d, J=8.3 Hz, 2H), 7.69 (d, J=8.1 Hz, 1H). MS m/z 477 (M+H)⁺.

### Example 15

### 4-(5-(4-((1-Methylpiperidin-4-yl)oxy)phenyl)-8-oxo-6-thioxo-5,7-diazaspiro[3.4]octan-7-yl)-2-(trifluoromethyl)benzonitrile, Compound 12

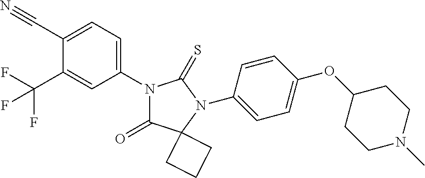 [SMILES: CN1CCC(Oc2ccc(N3C(=S)N(c4ccc(C#N)c(C(F)(F)F)c4)C(=O)C34CCC4)cc2)CC1]

Formaldehyde (37% wt in water, 0.32 mL, 4.23 mmol) was added to a solution of 4-(8-oxo-5-(4-(piperidin-4-yloxy)phenyl)-6-thioxo-5,7-diazaspiro[3.4]octan-7-yl)-2-(trifluoromethyl)benzonitrile (0.53 g, 1.06 mmol) in DCE (5 mL). The mixture was stirred at room temperature for 30 min, then sodium triacetoxyborohydride (0.71 g, 3.18 mmol) was added. The mixture was stirred overnight and diluted with EtOAc (20 mL). The solution was washed with 1M aqueous Na₂CO₃ (20 mL). The aqueous layer was extracted once with EtOAc (20 mL). The organic layer was dried over MgSO₄, filtered, and concentrated. The crude residue was purified by chromatography over silica gel (MeOH-dichloromethane gradient from 0% to 10%) to yield a foam that was triturated with Et₂O to give the product as a white solid (0.087 g, 16%)%). m.p. 99.3° C. ¹H NMR (300 MHz, DMSO-d₆) δ 1.44-1.61 (m, 1H), 1.61-1.78 (m, 2H), 1.86-2.06 (m, 3H), 2.17-2.34 (m, 2H), 2.24 (s, 3H), 2.34-2.47 (m, 2H), 2.54-2.65 (m, 2H), 2.65-2.78 (m, 2H), 4.35-4.58 (m, 1H), 7.14 (d, J=8.8 Hz, 2H), 7.30 (d, J=8.8 Hz, 2H), 8.05 (dd, J=8.3, 1.9 Hz, 1H), 8.25 (d, J=2.0 Hz, 1H), 8.37 (d, J=8.3 Hz, 1H). MS m/z 515.0 (M+H)⁺.

### Example 16

### 2-Chloro-4-(5-(4-((1-methylpiperidin-4-yl)oxy)phenyl)-8-oxo-6-thioxo-5,7-diazaspiro[3.4]octan-7-yl)benzonitrile, Compound 22

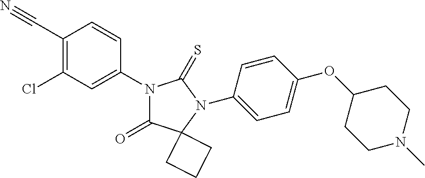 [SMILES: CN1CCC(Oc2ccc(N3C(=S)N(c4ccc(C#N)c(Cl)c4)C(=O)C34CCC4)cc2)CC1]

Formaldehyde (37% wt in water, 0.26 mL, 3.48 mmol) was added to a solution of 2-chloro-4-(8-oxo-5-(4-(piperidin-4-yloxy)phenyl)-6-thioxo-5,7-diazaspiro[3.4]octan-7-yl)benzonitrile (Example 12, STEP C) (0.406 g, 0.87 mmol) in DCE (5 mL). The mixture was stirred at room temperature for 30 min, then sodium triacetoxyborohydride (0.58 g, 2.61 mmol) was added. The reaction mixture was stirred overnight and diluted with EtOAc (20 mL). The solution was washed with 1M aqueous Na₂CO₃ solution (20 mL). The aqueous layer was extracted once with EtOAc (20 mL). The organic layer was dried over MgSO₄, filtered, and concentrated. The crude residue was purified by chromatography over silica gel (MeOH-DCM from gradient 0% to 10%) to yield the product as a white solid (197 mg, 46%). m.p. 152.2° C. ¹H NMR (300 MHz, Chloroform-d) δ 1.57-1.74 (m, 1H), 1.82-2.01 (m, 2H), 2.01-2.17 (m, 2H), 2.18-2.31 (m, 1H), 2.36 (s, 3H), 2.39-2.50 (m, 2H), 2.50-2.69 (m, 4H), 2.70-2.86 (m, 2H), 4.25-4.53 (m, 1H), 7.06 (d, J=8.9 Hz, 2H), 7.21 (d, J=8.9 Hz, 2H), 7.53 (dd, J=8.3, 1.9 Hz, 1H), 7.69 (d, J=1.9 Hz, 1H), 7.79 (d, J=8.4 Hz, 1H). MS m/z 481.0 (M+H)⁺.

### Example 17

### 4-(8-Oxo-5-(6-(piperidin-4-yloxy)pyridin-3-yl)-6-thioxo-5,7-diazaspiro[3.4]octan-7-yl)-2-(trifluoromethyl)benzonitrile hydrochloride (Compound 13)

### Step A: 5-Aminopyridin-2-ol, 17a

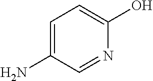

A solution of 5-nitropyridin-2-ol (150 g, 1.07 mol) in MeOH (2 L) was purged using nitrogen gas and vacuum. Palladium on charcoal (10% wet) was added and the mixture was hydrogenated (40 psi) for 16 h. The reaction mixture passed through diatomaceous earth and the filtrate concentrated under reduced pressure to give compound 17a as a dark oil, which was used directly into the next step.

### Step B: 1-((6-Hydroxypyridin-3-yl)amino)cyclobutane-1-carbonitrile, 17b

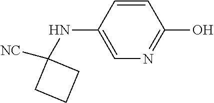 [SMILES: N#CC1(Nc2ccc(O)nc2)CCC1]

To a solution of 5-aminopyridin-2-ol (60 g, 490 mmol) and cyclobutanone (47.6 mL, 638 mmol) in MeOH (700 mL) was added zinc iodide (7.8 g, 24.43 mmol) at room temperature. Trimethylsilanecarbonitrile (73 g, 735.84 mmol) was added in several portions. The mixture was stirred at 50° C. for 16 h and then allowed to cool down to RT and concentrated under reduced pressure. The residue was purified by chromatography over silica gel (gradient of MeOH in DCM from 0 to 8%). The fractions with product were collected and concentrated under reduced pressure to yield 1-((6-hydroxypyridin-3-yl)amino)cyclobutane-1-carbonitrile as a dark solid (45 g, 48%). ¹H NMR (300 MHz, Chloroform-d) δ 1.93-2.10 (m, 2H) 2.18-2.32 (m, 2H) 2.55 (br. s., 2H) 5.77-5.92 (m, 1H) 6.26-6.39 (m, 1H) 6.48-6.67 (m, 1H) 6.99-7.19 (m, 1H) 10.81-11.19 (m, 1H). C₁₀H₁₁N₃O MS m/z 190.1 (M+H)⁺.

### Step C: 4-(5-(6-Hydroxypyridin-3-yl)-8-oxo-6-thioxo-5,7-diazaspiro[3.4]octan-7-yl)-2-(trifluoromethyl)benzonitrile, 17c

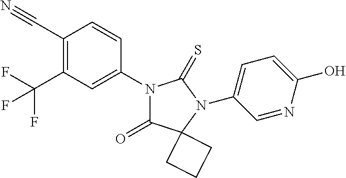 [SMILES: N#Cc1ccc(N2C(=O)C3(CCC3)N(c3ccc(O)nc3)C2=S)cc1C(F)(F)F]

A solution of 1-((6-hydroxypyridin-3-yl)amino)cyclobutane-1-carbonitrile (0.8 g, 4.23 mmol) and 4-isothiocyanato-2-trifluoromethyl-benzonitrile (1.16 g, 5.07 mmol) in DMA (6.7 mL) was heated to 60° C. and stirred at that temperature for 15 h. The mixture was allowed to cool to room temperature. MeOH (6.8 mL) and 1M aqueous HCl (6.8 mL) were added and the mixture was stirred at room temperature for 30 min. The crude reaction mixture was quenched with saturated aqueous NaHCO₃ solution and extracted with EtOAc. The organic layer was separated and washed with brine, then dried over MgSO₄, filtered, and concentrated. The residue was treated with hot acetonitrile, sonicated at 60° C. for 10 min, then cooled to room temperature. The precipitate was collected by filtration and washed with acetonitrile to afford compound 17c as a beige solid (0.900 g, 50.9%). MS m/z 418.9 (M+H)⁺.

### Step D: tert-Butyl 4-((5-(7-(4-cyano-3-(trifluoromethyl)phenyl)-8-oxo-6-thioxo-5,7-diazaspiro[3.4]octan-5-yl)pyridin-2-yl)oxy)piperidine-1-carboxylate, 17d

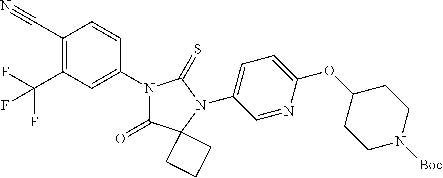 [SMILES: CC(C)(C)OC(=O)N1CCC(Oc2ccc(N3C(=S)N(c4ccc(C#N)c(C(F)(F)F)c4)C(=O)C34CCC4)cn2)CC1]

4-(5-(6-Hydroxypyridin-3-yl)-8-oxo-6-thioxo-5,7-diazaspiro[3.4]octan-7-yl)-2-(trifluoromethyl)benzonitrile (0.900 g, 2.15 mmol), tert-butyl 4-hydroxypiperidine-1-carboxylate (0.476 g, 0.237 mmol), and triphenylphosphine (1.24 g, 4.73 mmol) were dissolved in dry THF (8 mL) under a nitrogen atmosphere and heated at 50° C. A solution of diisopropyl azodicarboxylate (0.849 mL, 4.302 mmol) in THF (3 mL) was added dropwise over 15-20 min. Once the addition was complete, stirring was continued for 15 h at the same temperature. The mixture was allowed to cool to room temperature, then was poured onto aqueous NaHCO₃ solution and extracted with EtOAc. The organic layer was dried over MgSO₄, filtered, and concentrated to dryness. The residue was purified by flash chromatography over silica gel (EtOAc-heptane gradient from 5% to 30%). Pure fractions were combined, concentrated, and dried under high vacuum to give compound 17d (1.29 g, 99%). MS m/z 546.0 (M−56+H)⁺.

### Step E: 4-(8-Oxo-5-(6-(piperidin-4-yloxy)pyridin-3-yl)-6-thioxo-5,7-diazaspiro[3.4]-octan-7-yl)-2-(trifluoromethyl)benzonitrile hydrochloride, compound 13

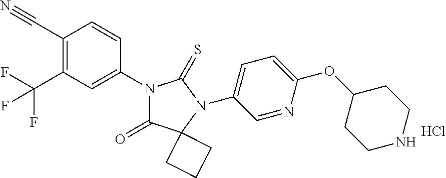 [SMILES: N#Cc1ccc(N2C(=O)C3(CCC3)N(c3ccc(OC4CCNCC4)nc3)C2=S)cc1C(F)(F)F ; Cl]

A 4N HCl solution in dioxane (5.4 mL) was added to a solution of tert-butyl 4-((5-(7-(4-cyano-3-(trifluoromethyl)phenyl)-8-oxo-6-thioxo-5,7-diazaspiro[3.4]octan-5-yl)pyridin-2-yl)oxy)piperidine-1-carboxylate (1.29 g, 2.15 mmol) in dichloromethane (21 mL) at 0° C. under a nitrogen atmosphere. The mixture was stirred at room temperature for 2 h, then was evaporated to dryness. The solid residue was crushed and triturated with a mixture of EtOAc and Et₂O. The resulting white solid was collected by filtration, washed with Et₂O and heptane, and dried under high vacuum to constant weight to give the product (1.12 g, 97%). ¹H NMR (300 MHz, DMSO-d₆) δ 1.45-1.68 (m, 1H), 1.78-2.07 (m, 3H), 2.13-2.31 (m, 2H), 2.34-2.47 (m, 2H), 2.58-2.74 (m, 2H), 3.01-3.21 (m, 2H), 3.21-3.37 (m, 2H), 5.18-5.42 (m, 1H), 7.07 (d, J=8.8 Hz, 1H), 7.81 (dd, J=8.8, 2.6 Hz, 1H), 8.05 (dd, J=8.3, 1.9 Hz, 1H), 8.17-8.29 (m, 2H), 8.38 (d, J=8.3 Hz, 1H), 8.95 (s, 2H). MS m/z 502.1 (M+H)⁺.

### Example 18

### 2-Chloro-4-(8-oxo-5-(6-(piperidin-4-yloxy)pyridin-3-yl)-6-thioxo-5,7-diazaspiro[3.4]octan-7-yl)benzonitrile hydrochloride (Compound 23)

### Step A: 2-Chloro-4-(5-(6-hydroxypyridin-3-yl)-8-oxo-6-thioxo-5,7-diazaspiro[3.4]octan-7-yl)benzonitrile, 18a

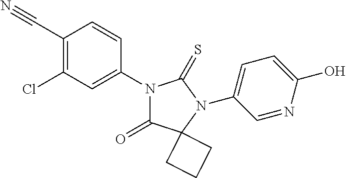 [SMILES: N#Cc1ccc(N2C(=O)C3(CCC3)N(c3ccc(O)nc3)C2=S)cc1Cl]

Compound 18a (filtered solid, 0.273 g, 17%, plus additional product recovered from mother liquors, 1.42 g) was prepared using the procedure of Example 17, STEP C, substituting 2-chloro-4-isothiocyanato-benzonitrile for 4-isothiocyanato-2-trifluoromethyl-benzonitrile. MS m/z 384.8 (M+H)⁺.

### Step B: tert-Butyl 4-((5-(7-(3-chloro-4-cyanophenyl)-8-oxo-6-thioxo-5,7-diazaspiro[3.4]octan-5-yl)pyridin-2-yl)oxy)piperidine-1-carboxylate, 18b

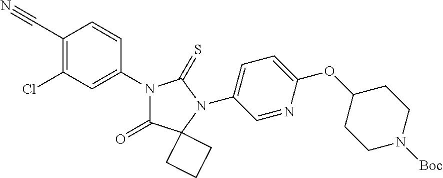 [SMILES: CC(C)(C)OC(=O)N1CCC(Oc2ccc(N3C(=S)N(c4ccc(C#N)c(Cl)c4)C(=O)C34CCC4)cn2)CC1]

The product (0.99 g, 41% purity) was prepared using the procedure of Example 17, STEP D, substituting 2-chloro-4-(5-(6-hydroxypyridin-3-yl)-8-oxo-6-thioxo-5,7-diazaspiro[3.4]octan-7-yl)benzonitrile for 4-(5-(6-Hydroxypyridin-3-yl)-8-oxo-6-thioxo-5,7-diazaspiro[3.4]octan-7-yl)-2-(trifluoromethyl)benzonitrile. MS m/z 512.0 (M−56+H)⁺.

### Step C: 2-Chloro-4-(8-oxo-5-(6-(piperidin-4-yloxy)pyridin-3-yl)-6-thioxo-5,7-diazaspiro[3.4]octan-7-yl)benzonitrile hydrochloride, Compound 23

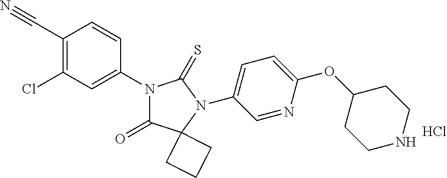 [SMILES: N#Cc1ccc(N2C(=O)C3(CCC3)N(c3ccc(OC4CCNCC4)nc3)C2=S)cc1Cl ; Cl]

The product (0.133 g, 37%) was prepared according to the procedure of Example 17, STEP E, substituting tert-butyl 4-((5-(7-(3-chloro-4-cyanophenyl)-8-oxo-6-thioxo-5,7-diazaspiro[3.4]octan-5-yl)pyridin-2-yl)oxy)piperidine-1-carboxylate for tert-butyl 4-((5-(7-(4-cyano-3-(trifluoromethyl)phenyl)-8-oxo-6-thioxo-5,7-diazaspiro[3.4]octan-5-yl)pyridin-2-yl)oxy)piperidine-1-carboxylate. m.p. 260.1° C. ¹H NMR (300 MHz, DMSO-d₆) δ 1.45-1.69 (m, 1H), 1.96 (d, J=9.3 Hz, 3H), 2.18 (s, 2H), 2.40 (q, J=10.5 Hz, 2H), 2.59 (d, J=10.3 Hz, 2H), 3.14 (d, J=9.3 Hz, 2H), 3.24-3.37 (m, 2H), 5.31 (s, 1H), 7.07 (d, J=8.8 Hz, 1H), 7.70 (dd, J=8.4, 1.9 Hz, 1H), 7.80 (dd, J=8.8, 2.6 Hz, 1H), 7.96 (d, J=1.9 Hz, 1H), 8.17 (d, J=8.4 Hz, 1H), 8.22 (d, J=2.5 Hz, 1H), 8.90 (s, 2H). MS m/z 468.1 (M+H)⁺.

### Example 19

### 4-(5-(6-((1-Methylpiperidin-4-yl)oxy)pyridin-3-yl)-8-oxo-6-thioxo-5,7-diazaspiro[3.4]-octan-7-yl)-2-(trifluoromethyl)benzonitrile hydrochloride (Compound 14)

### Step A: 4-(8-Oxo-5-(6-(piperidin-4-yloxy)pyridin-3-yl)-6-thioxo-5,7-diazaspiro[3.4]-octan-7-yl)-2-(trifluoromethyl)benzonitrile, 19a

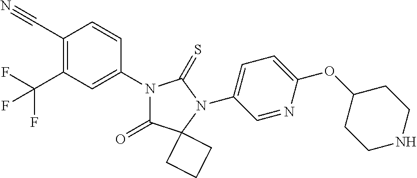 [SMILES: N#Cc1ccc(N2C(=O)C3(CCC3)N(c3ccc(OC4CCNCC4)nc3)C2=S)cc1C(F)(F)F]

4-(8-Oxo-5-(6-(piperidin-4-yloxy)pyridin-3-yl)-6-thioxo-5,7-diazaspiro[3.4]octan-7-yl)-2-(trifluoromethyl)benzonitrile hydrochloride (0.80 g) was dissolved in dichloromethane and washed with saturated aqueous NaHCO₃ solution. The organic layer was dried over MgSO₄, filtered, and concentrated to give compound 19a (0.701 g). MS m/z 502.1 (M+H)⁺.

### Step B: 4-(5-(6-((1-Methylpiperidin-4-yl)oxy)pyridin-3-yl)-8-oxo-6-thioxo-5,7-diazaspiro[3.4]octan-7-yl)-2-(trifluoromethyl)benzonitrile, 19b

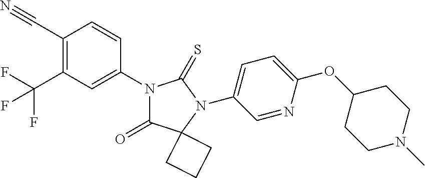 [SMILES: CN1CCC(Oc2ccc(N3C(=S)N(c4ccc(C#N)c(C(F)(F)F)c4)C(=O)C34CCC4)cn2)CC1]

Formaldehyde (37% wt in water, 0.156 mL, 2.09 mmol) was added to a solution of 4-(8-oxo-5-(6-(piperidin-4-yloxy)pyridin-3-yl)-6-thioxo-5,7-diazaspiro[3.4]octan-7-yl)-2-(trifluoromethyl)benzonitrile (0.350 g, 0.70 mmol) in DCE (29 mL). The mixture was stirred at room temperature for 5 min, then sodium triacetoxyborohydride (0.444 g, 2.09 mmol) was added. The reaction was stirred for 15 h and diluted with dichloromethane. The solution was washed with saturated aqueous NaHCO₃ solution. The organic layer was dried over MgSO₄, filtered, and concentrated to give crude compound 19b. The compound was purified by flash chromatography over silica gel (MeOH-dichloromethane gradient from 0% to 10%). Pure product fractions were combined and concentrated to dryness to afford the product as a beige solid (0.299 g, 83%). ¹H NMR (300 MHz, Chloroform-d) δ 1.61-1.82 (m, 1H), 1.84-2.01 (m, 2H), 2.06-2.31 (m, 3H), 2.34-2.37 (m, 4H), 2.51 (dt, J=12.8, 9.8 Hz, 3H), 2.60-2.72 (m, 2H), 2.76 (td, J=11.9, 11.3, 5.9 Hz, 2H), 5.15 (dt, J=8.4, 4.2 Hz, 1H), 6.90 (d, J=8.7 Hz, 1H), 7.51 (dd, J=8.8, 2.7 Hz, 1H), 7.84 (dd, J=8.4, 1.9 Hz, 1H), 7.93-8.01 (m, 2H), 8.09 (d, J=2.7 Hz, 1H). MS m/z 516.0 (M+H)⁺.

### Step C: 4-(5-(6-((1-Methylpiperidin-4-yl)oxy)pyridin-3-yl)-8-oxo-6-thioxo-5,7-diazaspiro[3.4]octan-7-yl)-2-(trifluoromethyl)benzonitrile hydrochloride, compound 14

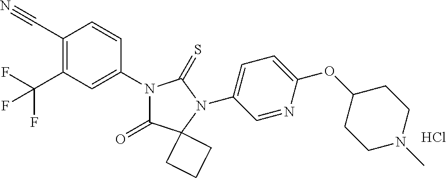 [SMILES: CN1CCC(Oc2ccc(N3C(=S)N(c4ccc(C#N)c(C(F)(F)F)c4)C(=O)C34CCC4)cn2)CC1 ; Cl]

The hydrochloride salt was prepared by addition of 4N HCl solution in dioxane to a solution of 4-(5-(6-((1-methylpiperidin-4-yl)oxy)pyridin-3-yl)-8-oxo-6-thioxo-5,7-diazaspiro[3.4]-octan-7-yl)-2-(trifluoromethyl)benzonitrile in EtOAc followed by the concentration of solvents. The white solid was crushed and triturated with heptane, collected by filtration, and vacuum-dried to constant weight to give the product (0.086 g, 95%). ¹H NMR (300 MHz, DMSO-d₆) δ 1.44-1.68 (m, 1H), 1.88-2.10 (m, 2H), 2.11-2.25 (m, 2H), 2.26-2.48 (m, 3H), 2.57-2.71 (m, 2H), 2.71-2.85 (m, 3H), 3.03-3.29 (m, 2H), 3.31-3.61 (m, 1H), 3.61-3.79 (m, 1H), 5.17-5.44 (m, 1H), 6.96-7.17 (m, 1H), 7.74-7.94 (m, 1H), 8.06 (d, J=8.3 Hz, 1H), 8.16-8.30 (m, 2H), 8.38 (d, J=8.3 Hz, 1H), 10.60-10.95 (m, 1H). MS m/z 516.0 (M+H)⁺.

### Example 20

### 2-Chloro-4-(5-(6-((1-methylpiperidin-4-yl)oxy)pyridin-3-yl)-8-oxo-6-thioxo-5,7-diazaspiro[3.4]octan-7-yl)benzonitrile hydrochloride (Compound 24)

### Step A: 2-Chloro-4-(8-oxo-5-(6-(piperidin-4-yloxy)pyridin-3-yl)-6-thioxo-5,7-diazaspiro[3.4]octan-7-yl)benzonitrile, 20a

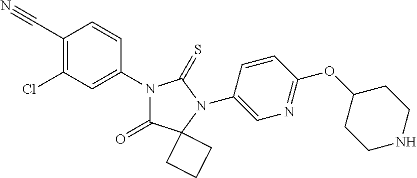 [SMILES: N#Cc1ccc(N2C(=O)C3(CCC3)N(c3ccc(OC4CCNCC4)nc3)C2=S)cc1Cl]

Using the procedure of Example 17, STEP E, substituting tert-butyl 4-((5-(7-(3-chloro-4-cyanophenyl)-8-oxo-6-thioxo-5,7-diazaspiro[3.4]octan-5-yl)pyridin-2-yl)oxy)piperidine-1-carboxylate for tert-butyl 4-((5-(7-(4-cyano-3-(trifluoromethyl)phenyl)-8-oxo-6-thioxo-5,7-diazaspiro[3.4]octan-5-yl)pyridin-2-yl)oxy)piperidine-1-carboxylate, the hydrochloride salt was isolated. This material was dissolved in dichloromethane and washed with saturated aqueous NaHCO₃ solution. The organic layer was dried and concentrated and the residue was purified by flash chromatography over silica gel (MeOH-dichloromethane gradient from 0% to 6%) to give compound 20a (0.795 g, 48%). MS m/z 467.9 (M+H)⁺.

### Step B: 2-Chloro-4-(5-(6-((1-methylpiperidin-4-yl)oxy)pyridin-3-yl)-8-oxo-6-thioxo-5,7-diazaspiro[3.4]octan-7-yl)benzonitrile, 20b

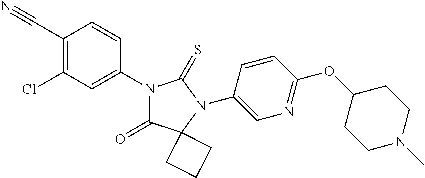 [SMILES: CN1CCC(Oc2ccc(N3C(=S)N(c4ccc(C#N)c(Cl)c4)C(=O)C34CCC4)cn2)CC1]

The product (0.308 g) was prepared using the procedure of Example 19, STEP B, substituting 2-chloro-4-(8-oxo-5-(6-(piperidin-4-yloxy)pyridin-3-yl)-6-thioxo-5,7-diazaspiro[3.4]octan-7-yl)benzonitrile for 4-(8-oxo-5-(6-(piperidin-4-yloxy)pyridin-3-yl)-6-thioxo-5,7-diazaspiro[3.4]octan-7-yl)-2-(trifluoromethyl)benzonitrile. m.p. 260.1° C. ¹H NMR (300 MHz, Chloroform-d) δ 1.61-1.75 (m, 1H), 1.82-1.97 (m, 2H), 2.05-2.27 (m, 3H), 2.35 (s, 3H), 2.37-2.55 (m, 4H), 2.58-2.71 (m, 2H), 2.71-2.86 (m, 2H), 5.03-5.24 (m, 1H), 6.88 (d, J=8.8 Hz, 1H), 7.44-7.55 (m, 2H), 7.68 (d, J=1.9 Hz, 1H), 7.78 (d, J=8.4 Hz, 1H), 8.07 (d, J=2.6 Hz, 1H). MS m/z 482.0 (M+H)⁺.

### Step C: 2-Chloro-4-(5-(6-((1-methylpiperidin-4-yl)oxy)pyridin-3-yl)-8-oxo-6-thioxo-5,7-diazaspiro[3.4]octan-7-yl)benzonitrile hydrochloride, compound 24

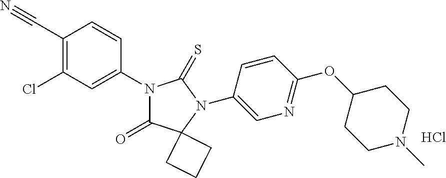 [SMILES: CN1CCC(Oc2ccc(N3C(=S)N(c4ccc(C#N)c(Cl)c4)C(=O)C34CCC4)cn2)CC1 ; Cl]

The product (0.190 g) was prepared using the procedure of Example 19, STEP C, substituting 2-chloro-4-(5-(6-((1-methylpiperidin-4-yl)oxy)pyridin-3-yl)-8-oxo-6-thioxo-5,7-diazaspiro[3.4]octan-7-yl)benzonitrile for 4-(5-(6-((1-methylpiperidin-4-yl)oxy)pyridin-3-yl)-8-oxo-6-thioxo-5,7-diazaspiro[3.4]octan-7-yl)-2-(trifluoromethyl)benzonitrile. m.p. 239.0° C. ¹H NMR (300 MHz, DMSO-d₆) δ 1.47-1.68 (m, 1H), 1.86-2.12 (m, 2H), 2.14-2.49 (m, 5H), 2.55-2.67 (m, 2H), 2.70-2.84 (m, 3H), 3.04-3.27 (m, 2H), 3.41-3.53 (m, 2H), 5.17-5.42 (m, 1H), 7.01-7.12 (m, 1H), 7.70 (dd, J=8.4, 1.9 Hz, 1H), 7.76-7.87 (m, 1H), 7.97 (d, J=1.9 Hz, 1H), 8.17 (d, J=8.4 Hz, 1H), 8.20-8.28 (m, 1H), 10.66-11.19 (m, 1H). MS m/z 481.9 (M+H)⁺.

### Example 21

### 2-Methyl-4-(5-(6-((1-methylpiperidin-4-yl)oxy)pyridin-3-yl)-8-oxo-6-thioxo-5,7-diazaspiro[3.4]octan-7-yl)benzonitrile (Compound 18)

### Step A: 1-((6-Methoxypyridin-3-yl)amino)cyclobutane-1-carbonitrile, 21a

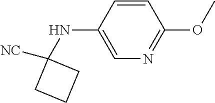

To a solution of 6-methoxypyridin-3-amine (5 g, 40.2 mmol) and cyclobutanone (4.2 g, 60.3 mmol) in MeOH (700 mL) in 1:1 AcOH/EtOH (80 mL), in a system furnished with an aqueous NaOH trap, was added NaCN (2.96 g, 60.3 mmol). The mixture was stirred at room temperature for 4 h, then poured onto ice water. The mixture was extracted with EtOAc and the combined organic layers were washed with water and brine, dried over Na₂SO₄, and concentrated under reduced pressure. The residue was purified by column chromatography over silica gel (EtOAc-hexanes gradient from 0% to 50%) to give compound 21a (7.2 g). ¹H NMR (400 MHz, DMSO-d₆) δ 1.98-2.11 (m, 2H), 2.28-2.36 (m, 2H), 2.64-2.72 (m, 2H), 3.75 (s, 3H), 6.38 (s, 1H), 6.71 (d, J=8.8 Hz, 1H), 7.08 (dd, J=8.8, 3.0 Hz, 1H), 7.48 (d, J=2.5 Hz, 1H). MS m/z 204 (M+H)⁺.

### Step B: 4-(5-(6-Methoxypyridin-3-yl)-8-oxo-6-thioxo-5,7-diazaspiro[3.4]octan-7-yl)-2-methylbenzonitrile, 21b

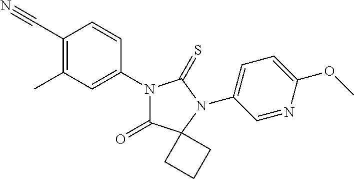 [SMILES: COc1ccc(N2C(=S)N(c3ccc(C#N)c(C)c3)C(=O)C23CCC3)cn1]

A solution of 1-((6-methoxypyridin-3-yl)amino)cyclobutane-1-carbonitrile (0.175 mg, 0.86 mmol) and 4-isothiocyanato-2-methylbenzonitrile (0.15 g, 0.86 mmol) in DMA (3.5 mL) was heated at 60° C. overnight. MeOH (5 mL) and and 2M aqueous HCl (5 mL) were added and the mixture was stirred at 90° C. for 2 h. The mixture was cooled to room temperature and partitioned between EtOAc and brine. The organic layer was concentrated and purified by flash chromatography over silica gel (EtOAc-hexanes gradient from 0% to 50%) to give compound 21b as a yellow foam (0.265 g).

### Step C: 4-(5-(6-Hydroxypyridin-3-yl)-8-oxo-6-thioxo-5,7-diazaspiro[3.4]octan-7-yl)-2-methylbenzonitrile, 21c

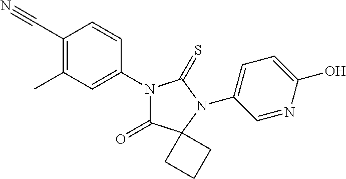 [SMILES: Cc1cc(N2C(=O)C3(CCC3)N(c3ccc(O)nc3)C2=S)ccc1C#N]

A 4 M HCl in dioxane solution (1 mL, 4 mmol) was added to a solution of 4-(5-(6-methoxypyridin-3-yl)-8-oxo-6-thioxo-5,7-diazaspiro[3.4]octan-7-yl)-2-methylbenzonitrile (0.265 g, 0.65 mmol) in dioxane (1 mL) and the mixture was heated at 65° C. overnight. After cooling, the mixture was concentrated, dissolved in MeOH, and purified by flash chromatography over silica gel (MeOH-dichloromethane gradient from 0% to 20%) to give compound 21c (0.182 g). MS m/z 365 (M+H)⁺.

### Step D: 2-Methyl-4-(5-(6-((1-methylpiperidin-4-yl)oxy)pyridin-3-yl)-8-oxo-6-thioxo-5,7-diazaspiro[3.4]octan-7-yl)benzonitrile, compound 18

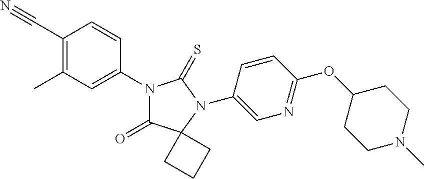 [SMILES: Cc1cc(N2C(=O)C3(CCC3)N(c3ccc(OC4CCN(C)CC4)nc3)C2=S)ccc1C#N]

4-(5-(6-Hydroxypyridin-3-yl)-8-oxo-6-thioxo-5,7-diazaspiro[3.4]octan-7-yl)-2-methylbenzonitrile (0.106 g, 0.29 mmol), 1-methyl-4-hydroxypiperidine (0.66 g, 0.58 mmol), triphenylphosphine (0.152 g, 0.58 mmol), and diisopropyl azodicarboxylate (0.11 mL, 0.58 mmol) were dissolved in dry THF (1 mL) under a nitrogen atmosphere and stirred at room temperature. The reaction mixture was concentrated under reduced pressure, adsorbed onto silica gel under reduced pressure, and purified by flash chromatography over silica gel (EtOAc-heptane gradient from 10% to 100%, followed by MeOH-dichloromethane gradient from 0% to 20%). Fractions containing product were combined and further purified by reversed phase HPLC. Pure fractions were combined and partitioned between organic solvent and saturated aqueous NaHCO₃. The organic layer was concentrated to give compound 18 (0.44 g, 99%). ¹H NMR (400 MHz, Chloroform-d) δ ppm 1.56-1.82 (m, 2H), 1.82-2.00 (m, 2H), 2.04-2.15 (m, 2H), 2.15-2.33 (m, 2H), 2.36 (br s, 3H), 2.40-2.55 (m, 2H), 2.62 (s, 3H), 2.61-2.71 (m, 2H), 2.78 (br s, 2H), 5.15 (br s, 1H), 6.90 (d, J=8.8 Hz, 1H), 7.38 (d, J=8.5 Hz, 1H), 7.42 (s, 1H), 7.52 (dd, J=8.7, 2.4 Hz, 1H), 7.75 (d, J=8.3 Hz, 1H), 8.10 (d, J=2.3 Hz, 1H). MS m/z 462 (M+H)⁺.

### Example 22

### 2-Methoxy-4-(5-(6-((1-methylpiperidin-4-yl)oxy)pyridin-3-yl)-8-oxo-6-thioxo-5,7-diazaspiro[3.4]octan-7-yl)benzonitrile (Compound 20)

### Step A: 2-Methoxy-4-(5-(6-methoxypyridin-3-yl)-8-oxo-6-thioxo-5,7-diazaspiro[3.4]-octan-7-yl)benzonitrile, 22a

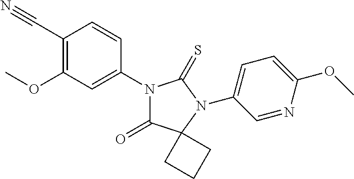 [SMILES: COc1ccc(N2C(=S)N(c3ccc(C#N)c(OC)c3)C(=O)C23CCC3)cn1]

The product (0.294 g) was prepared using the procedure of Example 21, STEP B, substituting 4-isothiocyanato-2-methoxybenzonitrile for 4-isothiocyanato-2-methylbenzonitrile.

### Step B: 4-(5-(6-Hydroxypyridin-3-yl)-8-oxo-6-thioxo-5,7-diazaspiro[3.4]octan-7-yl)-2-methoxybenzonitrile, 22b

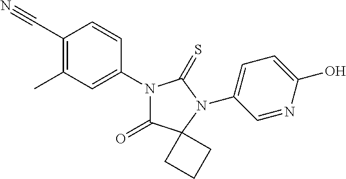 [SMILES: Cc1cc(N2C(=O)C3(CCC3)N(c3ccc(O)nc3)C2=S)ccc1C#N]

The product (0.244 g) was prepared using the procedure of Example 21, STEP C, substituting 2-methoxy-4-(5-(6-methoxypyridin-3-yl)-8-oxo-6-thioxo-5,7-diazaspiro[3.4]-octan-7-yl)benzonitrile for 4-(5-(6-methoxypyridin-3-yl)-8-oxo-6-thioxo-5,7-diazaspiro-[3.4]octan-7-yl)-2-methylbenzonitrile. MS m/z 381 (M+H)⁺.

### Step C: 2-Methoxy-4-(5-(6-((1-methylpiperidin-4-yl)oxy)pyridin-3-yl)-8-oxo-6-thioxo-5,7-diazaspiro[3.4]octan-7-yl)benzonitrile, compound 20

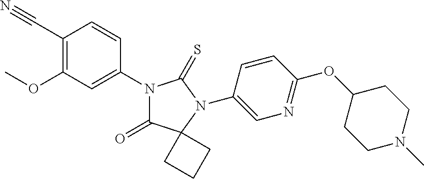 [SMILES: COc1cc(N2C(=O)C3(CCC3)N(c3ccc(OC4CCN(C)CC4)nc3)C2=S)ccc1C#N]

The product (0.063 g) was prepared using the procedure of Example 21, STEP D, substituting 4-(5-(6-hydroxypyridin-3-yl)-8-oxo-6-thioxo-5,7-diazaspiro[3.4]octan-7-yl)-2-methoxybenzonitrile for 4-(5-(6-hydroxypyridin-3-yl)-8-oxo-6-thioxo-5,7-diazaspiro[3.4]-octan-7-yl)-2-methylbenzonitrile. ¹H NMR (400 MHz, Chloroform-d) δ ppm 1.71 (q, J=10.4 Hz, 2H), 1.83-2.01 (m, 2H), 2.13 (br s, 2H), 2.16-2.33 (m, 2H), 2.36 (s, 3H), 2.41-2.56 (m, 2H), 2.62-2.74 (m, 2H), 2.78 (br s, 2H), 3.97 (s, 3H), 5.15 (br s, 1H), 6.91 (d, J=8.6 Hz, 1H), 7.09 (s, 1H), 7.11 (d, J=8.1 Hz, 1H), 7.53 (dd, J=8.7, 2.1 Hz, 1H), 7.69 (d, J=8.1 Hz, 1H), 8.09-8.12 (m, 1H). MS m z 478 (M+H)⁺.

### Example 23

### 2-4-(8-Oxo-5-(2-(piperidin-4-yloxy)pyrimidin-5-yl)-6-thioxo-5,7-diazaspiro[3.4]octan-7-yl)-2-(trifluoromethyl)benzonitrile (Compound 15)

### Step A: tert-Butyl 4-((5-((1-cyanocyclobutyl)amino)pyrimidin-2-yl)oxy)piperidine-1-carboxylate, 23a

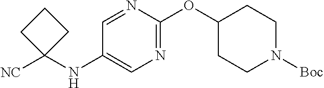 [SMILES: CC(C)(C)OC(=O)N1CCC(Oc2ncc(NC3(C#N)CCC3)cn2)CC1]

Cyclobutanone (6.2 mL, 82.8 mmol) and sodium cyanide (4.06 g, 82.8 mmol) were added successively to a solution of tert-butyl 4-((5-aminopyrimidin-2-yl)oxy)piperidine-1-carboxylate (Example 9, STEP B) (12.2 g, 41.4 mmol) in acetic acid (200 mL). The reaction mixture was stirred overnight at room temperature. The solution was then concentrated under reduced pressure in a fume hood. The residue was diluted with EtOAc (200 mL) and washed with 1M aqueous Na₂CO₃ solution (100 mL) and brine (100 mL). The organic layer was dried over MgSO₄, filtered, and concentrated to a crude oily residue. Chromatography over silica gel (EtOAc-heptane gradient from 0% to 60%) gave pure compound 23a (12.6 g, 81%). ¹H NMR (300 MHz, Chloroform-d) δ 1.47 (s, 9H), 1.72-1.86 (m, 2H), 1.91-2.07 (m, 2H), 2.12-2.31 (m, 2H), 2.31-2.45 (m, 2H), 2.68-2.88 (m, 2H), 3.17-3.37 (m, 2H), 3.70-3.91 (m, 3H), 5.00-5.16 (m, 1H), 8.05 (s, 2H). C₁₉H₂₇N₅O₃ MS m/z 374 (M+H)⁺.

### Step B: tert-Butyl 4-((5-(7-(4-cyano-3-(trifluoromethyl)phenyl)-8-oxo-6-thioxo-5,7-diazaspiro[3.4]octan-5-yl)pyrimidin-2-yl)oxy)piperidine-1-carboxylate, 23b

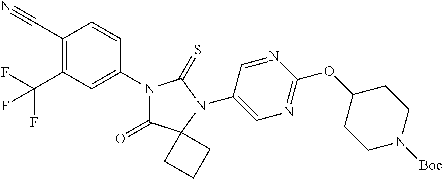 [SMILES: CC(C)(C)OC(=O)N1CCC(Oc2ncc(N3C(=S)N(c4ccc(C#N)c(C(F)(F)F)c4)C(=O)C34CCC4)cn2)CC1]

tert-Butyl 4-((5-((1-cyanocyclobutyl)amino)pyrimidin-2-yl)oxy)piperidine-1-carboxylate (0.250 g, 6.61 mmol), and freshly prepared 4-isothiocyanato-2-trifluoromethyl-benzonitrile (0.342 g, 1.50 mmol) were heated at 60° C. in DMA (10 mL) for 2 h. The reaction mixture was recharged with 4-isothiocyanato-2-trifluoromethyl-benzonitrile (0.342 g, 1.50 mmol) and heating was continued at 60° C. overnight. The mixture was then allowed to cool to room temperature and MeOH (2 mL) and 1M aqueous HCl (2 mL) were added. The stirring was maintained at room temperature for 1 h. EtOAc (50 mL) was added and the resulting solution was washed with 1M aqueous Na₂CO₃ solution (150 mL). The organic layer was dried over MgSO₄, filtered, and concentrated to dryness. The crude material was purified by chromatography over silica gel (EtOAc-heptane gradient from 0 to 50%). The fractions with product were collected and concentrated under reduced pressure to yield an amorphous solid (0.331 g, 74%). ¹H NMR (300 MHz, Chloroform-d) δ 1.48 (s, 9H), 1.68-1.81 (m, 1H), 1.82-1.98 (m, 2H), 1.98-2.14 (m, 2H), 2.20-2.37 (m, 1H), 2.38-2.55 (m, 2H), 2.65-2.86 (m, 2H), 3.23-3.42 (m, 2H), 3.76-3.91 (m, 2H), 5.16-5.37 (m, 1H), 7.83 (dd, J=8.3, 2.1 Hz, 1H), 7.90-8.06 (m, 2H), 8.50 (s, 2H). MS m/z 546.9 (M+H−tBu)⁺.

### Step C: 2-4-(8-Oxo-5-(2-(piperidin-4-yloxy)pyrimidin-5-yl)-6-thioxo-5,7-diazaspiro[3.4]octan-7-yl)-2-(trifluoromethyl)benzonitrile, compound 15

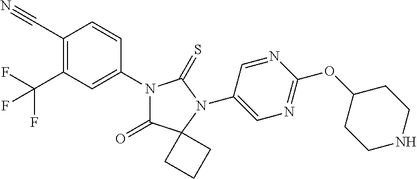 [SMILES: N#Cc1ccc(N2C(=O)C3(CCC3)N(c3cnc(OC4CCNCC4)nc3)C2=S)cc1C(F)(F)F]

TFA (1 mL) was added to a stirring solution of tert-butyl 4-((5-(7-(4-cyano-3-(trifluoromethyl)phenyl)-8-oxo-6-thioxo-5,7-diazaspiro[3.4]octan-5-yl)pyrimidin-2-yl)oxy)piperidine-1-carboxylate (0.331 g, 0.549 mmol) in DCM (5 mL) with stirring. The mixture was stirred for 2 h at room temperature and then concentrated under reduced pressure. The residue was co-evaporated with toluene (3×15 mL) under reduced pressure. The crude oily residue was purified by column chromatography over silica gel (MeOH-dichloromethane from 0% to 10%). The pure fractions were collected and concentrated. Trituration with Et₂O gave the product as a white solid (0.270 g, 95%). m.p. 137.4° C. ¹H NMR (300 MHz, DMSO-d₆) δ 1.51-1.73 (m, 1H), 1.86-2.08 (m, 2H), 2.12-2.31 (m, 2H), 2.37-2.49 (m, 2H), 2.56-2.71 (m, 2H), 3.09-3.23 (m, 2H), 3.24-3.33 (m, 2H), 5.19-5.41 (m, 1H), 8.05 (dd, J=8.4, 2.0 Hz, 1H), 8.23 (d, J=1.9 Hz, 1H), 8.40 (d, J=8.3 Hz, 1H), 8.63 (br s, 2H), 8.74 (s, 2H). MS m/z 503.0 (M+H)⁺.

### Example 24

### 4-(5-(2-((1-Methylpiperidin-4-yl)oxy)pyrimidin-5-yl)-8-oxo-6-thioxo-5,7-diazaspiro[3.4]octan-7-yl)-2-(trifluoromethyl)benzonitrile (Compound 16)

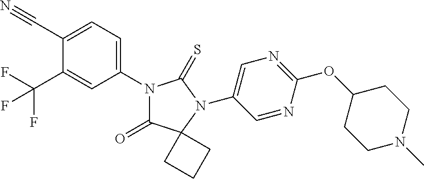 [SMILES: CN1CCC(Oc2ncc(N3C(=S)N(c4ccc(C#N)c(C(F)(F)F)c4)C(=O)C34CCC4)cn2)CC1]

Formaldehyde (37% wt in water, 0.097 mL, 1.29 mmol) was added to a solution of 2-4-(8-oxo-5-(2-(piperidin-4-yloxy)pyrimidin-5-yl)-6-thioxo-5,7-diazaspiro[3.4]octan-7-yl)-2-(trifluoromethyl)benzonitrile (Example 23, STEP C) (0.162 g, 0.322 mmol) in DCE (5 mL). The mixture was stirred at room temperature for 30 min, before sodium triacetoxyborohydride (0.216 g, 0.967 mmol) was added. The reaction was continued overnight and then diluted with EA (25 mL). The solution was washed with 1M aqueous Na₂CO₃ solution (10 mL). The aqueous phase was extracted with EA (20 mL). The combined organic layers were dried over MgSO₄, filtered, and concentrated to give the crude product. Chromatography over silica gel (MeOH-DCM from gradient from 0% to 10%) gave an amorphous solid. Trituration with Et₂O afforded a white solid (0.083 g, 47%). m.p. 161.9° C. ¹H NMR (300 MHz, Chloroform-d) δ 1.71-1.85 (m, 1H), 1.92-2.13 (m, 2H), 2.14-2.36 (m, 4H), 2.39 (s, 3H), 2.42-2.60 (m, 3H), 2.68-2.80 (m, 2H), 2.80-2.95 (m, 2H), 5.05-5.29 (m, 1H), 7.83 (dd, J=8.4, 2.0 Hz, 1H), 7.90-8.04 (m, 2H), 8.50 (s, 2H). MS m/z 517.0 (M+H)⁺.

### BIOLOGICAL EXAMPLES

The term “biological sample”, as used herein, includes, without limitation, cell cultures or extracts thereof, biopsied material obtained from a mammal or extracts thereof; and blood, saliva, urine, feces, semen, tears, or other body fluids or extracts thereof.

Antagonism of receptors in a biological sample is useful for a variety of purposes that are known to one of skill in the art. Examples of such purposes include, but are not limited to, biological assays, gene expression studies, and biological target identification.

Certain embodiments of the present invention are directed to a method of treatment by antagonizing AR in a patient or a subject in need of such treatment comprising the step of administering to said patient a compound of Formula (I) of the present invention, or a composition comprising said compound.

The activity of a compound of Formula (I) as an antagonist of AR or for the treatment of an AR-mediated disease, disorder or condition, may be assayed in vitro or in vivo. An in vivo assessment of the efficacy of the compounds of the invention may be made using an animal model of an AR-mediated disease, disorder or condition, e.g., a rodent or primate model. The in vivo assessment may be further defined as an androgen dependent organ development (Hershberger) assay or as a tumor xenograft model. Cell-based assays may be performed using, e.g., a cell line isolated from a tissue that expresses either wild type or mutant AR. Additionally, biochemical or mechanism based assays, e.g., transcription assays using a purified protein, Northern blot, RT-PCR, etc., may be performed.

In vitro assays include assays that determine cell morphology, protein expression, and/or the cytotoxicity, enzyme inhibitory activity, and/or the subsequent functional consequences of treatment of cells with compounds of the invention. Alternate or additional in vitro assays may be used to quantitate the ability of the inhibitor to bind to protein or nucleic acid molecules within the cell.

Inhibitor binding may be measured by radiolabelling the inhibitor prior to binding, isolating the inhibitor/target molecule complex and determining the amount of radiolabel bound. Alternatively or additionally, inhibitor binding may be determined by running a competition experiment where new inhibitors are incubated with purified proteins or nucleic acids bound to known radioligands. Detailed conditions of exemplary systems for assaying a compound of Formula (I) of the present invention as an antagonist of AR are set forth in the Biological Examples below.

Such assays are exemplary and not intended to limit the scope of the invention. The skilled practitioner can appreciate that modifications can be made to conventional assays to develop equivalent or other assays that can be employed to comparably assess activity or otherwise characterize compounds and/or compositions as described herein.

### In Vitro Assays

### Biological Example 1

### Antagonism of AR (WT or F876L) Reporter Assay

LNCaP AR (cs) and LNCaP F876L luciferase cell lines were generated by transduction of each cell line (description of cell line generation Joseph J D, Lu N, Qian J, Sensintaffar J, Shao G, Brigham D, Moon M, Maneval E C, Chen I, Darimont B, Hager J H. A clinically relevant androgen receptor mutation confers resistance to second-generation antiandrogens enzalutamide and ARN-509. Cancer Discov 2013; 3:1020-1029) with an Androgen Response Element Firefly Luciferase lentiviral construct at an MOI (multiplicity of infection) of 50 following the manufacturer's instructions (Qiagen). A stable pooled-population cell line was generated using puromycin (Life Technologies) selection at 1:10,000 v/v. The protocol below was used for both cell lines and for testing of the compounds of Formula (I) of the present invention.

LNCaP cells were grown to about 80% confluence, media removed and cells rinsed in Hank's balanced salt solution prior to separation from the plate with 0.05% Trypsin EDTA. Cells were lifted and trypsin negated in complete CSS (charcoal stripped serum) culture media. CSS was maintained on cells for 24 h prior to assay, at which time 5,000 cells/20 μL were seeded in Greiner 384 well White/White Tissue Culture Treated Plates and incubated for a further 1-2 hours at 37° C., 5% CO₂, prior to addition of 10 μL of 4× Test Compounds (compounds described herein) or Assay Controls (all diluted in complete media containing 10% css). A further 10 μL of 4×R-1881 Agonist Challenge (antagonist assay) or Buffer (agonist assay) was then added (all diluted in complete media containing 10% css). Agonist challenge was at 400 pM for WT assay and 600 pM for F876L assay. Plates containing cells and compounds herein were incubated for a further 20-24 hours at 37° C., 5% CO₂ before addition of 40 μL/well of Steady-Glo Luciferase Assay System Reagent (Promega# E2520). After 1 h, plates were read for luminescence on a BMG Pherastar.

Agonist Challenge:

R-1881 (Metribolone)—Agonist

Antagonist Control (Low Control):

5-(5-(4-((1-Methylpiperidin-4-yl)oxy)phenyl)-8-oxo-6-thioxo-5,7-diazaspiro[3.4]octan-7-yl)-3-(trifluoromethyl)picolinonitrile (WO 2011/103202, EXAMPLE 19, Compound 129, CAS #1332390-06-3).

### Calculations and Formulae:

RLU results were collected from the Pherastar and used directly for data calculation.

### Percent Max & Inhibition Calculated for Assays:

(1−(Sample RLU−Ave Low Control RLU[10 μM Antagonist Control])/(Ave High Control RLU [400 pM R-1881]−Ave Low Control RLU[10 μM Antagonist Control]))*100. % Inhibition:

((Sample RLU−Ave Low Control RLU[DMSO/Buffer])/(Ave High Control RLU [1 μM R-1881]−Ave Low Control RLU[DMSO/Buffer]))*100. % of 1 M R-1881 Agonist Max:

EC/IC50 calculations were achieved utilizing calculated RLU data and data fitting macros. Data were fit using least-squares methods to the following formula:

$${Y}_{[ fit ]} = {Y}_{[ low cmpd ]} + \frac{( {Y}_{[ high cmpd ]} - {Y}_{[ low cmpd ]} ) * {Y}_{[ cmpd ]}^{Hill}}{{Y}_{[ cmpd ]}^{Hill} + IC {50}^{Hill}}$$

wherein

Y_{[low cmpd]}=Y value with inactive compound

Y_{[high cmpd]}=Y value with fully active compound effector

Hill=Hill coefficient

EC/IC₅₀=concentration of compound with 50% effect

Resultant data are shown in Table 2.

TABLE 2

| | LNCaP-AR- | | LNCaP-AR- | | LNCaP-AR- | | LNCaP-AR- | |
|---|---|---|---|---|---|---|---|---|
| | wt ANT | | wt AG | | F876L ANT | | F876L AG | |
| | | MAX | | MAX | | MAX | | MAX |
| Cpd | pIC₅₀ | % Inh | pEC₅₀ | % Stim | pIC₅₀ | % Inh | pEC₅₀ | % Stim |
| 1 | 7.12 | 105.7 | <4.82 | 0.6 | 7.38 | 102.2 | <4.82 | −0.1 |
| 2 | 7.01 | 103.8 | <4.82 | −0.2 | 7.16 | 100.6 | <4.82 | 0.1 |
| 3 | 7.33 | 103.1 | <4.82 | 0.5 | 7.39 | 101.6 | <4.82 | 0.3 |
| 4 | 7.05 | 102.4 | <4.82 | 0.1 | 7.08 | 101.8 | <4.82 | 0.1 |
| 5 | 6.77 | 101.2 | <4.82 | 0.1 | 6.64 | 101.1 | <4.82 | 0.0 |
| 6 | 7.04 | 102.3 | <4.82 | 0.8 | 6.87 | 100.8 | <4.82 | −0.1 |
| 7 | 7.21 | 100.9 | <4.82 | 0.0 | 7.42 | 101.4 | <4.82 | −0.1 |
| 8 | 7.09 | 102.8 | <4.82 | 0.1 | 7.23 | 100.7 | <4.82 | 0.1 |
| 9 | 7.24 | 101.8 | <4.82 | −0.2 | 7.41 | 100.3 | <4.82 | 0.0 |
| 10 | 7.20 | 101.7 | <4.82 | 0.1 | 7.07 | 100.8 | <4.82 | 0.1 |
| 11 | 6.48 | 105.1 | <4.82 | 0.1 | 6.97 | 102.0 | <4.82 | 0.1 |
| 12 | 6.49 | 103.7 | <4.82 | 0.4 | 6.78 | 101.2 | <4.82 | 0.2 |
| 13 | 7.20 | 103.8 | <4.82 | 0.5 | 7.24 | 101.8 | <4.82 | 0.0 |
| 14 | 7.05 | 103.7 | <4.82 | 0.2 | 7.24 | 101.2 | <4.82 | 0.1 |
| 15 | 6.80 | 101.9 | <4.82 | −0.2 | 6.97 | 100.8 | <4.82 | −0.1 |
| 16 | 6.68 | 102.5 | <4.82 | −0.1 | 6.78 | 100.7 | <4.82 | 0.1 |
| 17 | 6.77 | 104.9 | <4.82 | −0.1 | 6.89 | 100.7 | <4.82 | 0.2 |
| 18 | 7.44 | 99.4 | <4.82 | 0.6 | 6.55 | 99.8 | <4.82 | 2.6 |
| 19 | 6.85 | 105.1 | <4.82 | −0.1 | 7.08 | 101.2 | <4.82 | 0.3 |
| 20 | 6.67 | 100.5 | <4.82 | −0.1 | 6.84 | 100.0 | <4.82 | 0.3 |
| 21 | 6.48 | 105.5 | <4.82 | 0.1 | 6.70 | 103.6 | <4.82 | 0.2 |
| 22 | 6.61 | 98.4 | <4.82 | −0.2 | 6.95 | 99.5 | <4.82 | 0.0 |
| 23 | 7.17 | 105.3 | <4.82 | 0.9 | 7.30 | 103.2 | <4.82 | 0.3 |
| 24 | 7.02 | 101.9 | <4.82 | 0.3 | 7.11 | 99.8 | <4.82 | 0.1 |

As used herein:
pIC₅₀ is defined as −Log₁₀(IC₅₀ expressed in [Molar]).
pEC₅₀ is defined as −Log₁₀(EC₅₀ expressed in [Molar]).
MAX % Inh is defined as the maximum % inhibition of R1881 control response observed for a compound over the tested concentration range.
MAX % Stim is defined as the maximum % stimulation (agonist response) observed for a compound over the tested concentration range.
LNCaP-AR-wt ANT refers to the reporter assay using LNCaP cells stably transfected with the Androgen Response Element Firefly Luciferase lentiviral construct and wild-type Androgen Receptor (AR-wt) in Antagonist mode.
LNCaP-AR-wt AG refers to the reporter assay using LNCaP cells stably transfected with the Androgen Response Element Firefly Luciferase lentiviral construct and wild-type Androgen Receptor (AR-wt) in Agonist mode.
LNCaP-AR-F876L ANT refers to the reporter assay using LNCaP cells stably transfected with the Androgen Response Element Firefly Luciferase lentiviral construct and F876L mutant Androgen Receptor (AR-F876L) in Antagonist mode.
LNCaP-AR-F876L AG refers to the reporter assay using LNCaP cells stably transfected with the Androgen Response Element Firefly Luciferase lentiviral construct and F876L mutant Androgen Receptor (AR-F876L) in Agonist mode.

### Biological Example 2

### AR in Cell Western Assay

LNCaP cells (8,000/well) are plated in RPMI media containing 10% Charcoal Dextran Stripped Serum into plates coated with poly-d-lysine. After 24 h cells are treated with compound from 30 μM to 0.0003 μM. At 20 h post compound addition the cells were fixed (30% formaldehyde in PBS) for 20′. Cells are permeabilized in PBS 0.1% Triton (50 μL/well, three times for 5′ each) and blocked with LiCor blocking buffer (50 μL/well, 90′). The wells are then incubated overnight at 4° C. with the rabbit IgG androgen receptor antibody (AR-N20, Santa Cruz antibody) diluted 1:1000 in LiCor blocking buffer/0.1% Tween-20. Wells are washed with 0.1% Tween-20/PBS (50 L/well, 5′ each) and then incubated in goat anti-rabbit IRDye™ 800CW (1:1000) and DRAQ5 DNA dye (1:10,0000 for 5 mM stock) diluted in 0.2% Tween-20/0.01% SDS/LiCor blocking buffer in the dark (90′). Cells are washed (50 μL/well, 5′ each) in 0.1% Tween-20/PBS. Wash buffer is removed and plates were read using the LiCor Odyssey.

### Biological Example 3

### LNCaP AR Localization Assay

LNCaP cells are seeded on day 1 in plates and incubated overnight at 37° C. prior to addition of 20 μl pre-diluted compound or DMSO (basal, vehicle control). Plates are incubated at 37° C. for 1-2 hr before addition of 20 μl of ligand solution (antagonist mode, high control) or CSS medium (agonist mode, unstimulated control) and incubation of the cells for +/−24 hours.

Cells are fixed in 140 μL of 10% Formaldehyde (5% final) and plates incubated for 15-20 min at RT. 100 μL 100% ice cold Methanol (stored at −20° C.) is added to permeabilise the cells, antibody staining protocol initiated and plates prepared for imaging. Staining is performed using an indirect immunofluorescence assay: For AR, primary antibody is a specific mouse anti-AR antibody (ab49450, Abcam), followed by a secondary goat anti-mouse antibody, carrying an alexa 488 fluorophore; For PSA, primary antibody is a specific rabbit anti-PSA antibody (5365S, Cell Signaling Technology), followed by a secondary goat anti rabbit antibody, carrying an alexa 568 fluorophore. Cells are counterstained with Hoechst for the nucleus and Cellmask™ for the cytoplasm stain. Plates are washed and maintained in PBS at 4° C. until further processed.

Plates are imaged using the 20×W lens on the Opera (Perkin Elmer) and the following calculations are then applied to derive the reported data from this assay.

LC=median of the low control values=minimum translocation
=cells in CSS medium (0.5% DMSO) and showing minimum translocation
   HC=median of the high control values=maximum translocation
=cells in CSS medium containing 1 nM of R1881 ligand (0.5% DMSO)

% EFFECT=(sample−LC)/(HC−LC)*100

% CTL=% of high-controls=(sample/HC)*100

Several features are calculated but include:
Ratio_Nuc2Cell_AR_TotalIntBC.median: % of total AR in the nucleus calculated as “total nuclear AR intensity”/“total cellular AR intensity” on the single-cell level and then the median over all cells reported as well feature [% effect]
Cell_AR_MeanIntBC.median: AR levels in the whole cell [% effect]
Cyto_AR_meanIntBC.median: AR levels in cytoplasm [% effect]
Nuc_AR_MeanIntBC.median: AR levels in nucleus [% effect]
Cell_Rpt_MeanIntBC.median: PSA levels in whole cell [% effect]
CellCount_AllDetected: number of the cells

### Biological Example 4

### Prostate Cancer Cell Viability Assay-VCaP

VCaP cells were counted and seeded into black 384-well plates with clear bottoms at a concentration of 125,000 cells per mL in phenol red-free DMEM containing 10% Charcoal Stripped Serum. 16 μL of the suspension was added per well and incubated for 48 h to allow the cells to adhere. After 48 hours, a 12 point serial semilog dilution of each compound was added to the cells in 16 μL at a final concentration of 100 M to 0.0003 μM. The compounds of Formula (I) were also run in antagonist mode using 30 pM R1881 in which 8 μL of the compound was added to the cells followed by 8 μL of R1881. After 5 days of incubation at 37° C., 16 μL Of CellTiter-Glo (Promega) was added to the cells and the relative luminescence units (RLUs) of each well determined using the Envision. The percent stimulation and % inhibition were determined for each sample and plotted using GraphPad Prism. Resultant data are shown in Table 3.

TABLE 3

| Compound | IC₅₀ (μM) |
|---|---|
| 1 | 2.31 |
| 2 | NC |
| 3 | 4.86* |
| 4 | 1.66* |
| 5 | 13.17 |
| 6 | 18.37 |
| 7 | 1.44 |
| 8 | 3.48 |
| 9 | 1.68* |
| 10 | 5.54* |
| 11 | 1.06 |
| 12 | 21.01 |
| 13 | 0.54 |
| 14 | 13.11 |
| 15 | NC |
| 16 | 5.29 |
| 17 | 3.42 |
| 18 | 12.1 |
| 19 | 9.87 |
| 20 | 1.4 |
| 21 | 1.25* |
| 22 | 0.58* |
| 23 | 0.54* |
| 24 | NC |

*n = 1
NC = not calculated (poor curve fit)

### Biological Example 5

### LNCaP Proliferation Assays

LNCaP cells were expanded in RPMI 10% FBS in T150 flasks. The cells were dislodged with 0.25% Trypsin, washed in complete media, centrifuged (300 g, 3 min), and the supernatant aspirated. The cells were resuspended in RPMI phenol-red free media with 1% charcoal-stripped serum (CSS) and counted using a ViCELL (Beckman-Coulter). 7500 cells were added to each well of a white optical bottom 384-well plate and incubated for 2 days at 37° C. 5% CO₂. Compound dilutions were prepared in RPMI CSS using 50 mM stock solutions and added to the cells either alone (agonist mode) or in combination with 0.1 nM R1881 (antagonist mode). The plates were incubated for 4 days, followed by addition of CellTiter-Glo Luminescent Cell Viability kit reagent (Promega). The plates were placed on a shaker at 3000 rpm for 10 minutes and then read on an EnVision plate reader (Perkin Elmer) using Luminescence assay default settings. The data was analyzed, normalized to 0.1 nM R1881 stimulation, and plotted in GraphPad Prism. Resultant data are shown in Table 4.

TABLE 4

IC₅₀ (μM)

| Compound | LNCaP WT | LNCaP F876L |
|---|---|---|
| 1 | ND | ND |
| 2 | 1.37 | 1.31 |
| 3 | NC | NC |
| 4 | 0.46 | 0.85 |
| 5 | 1.04 | 0.95 |
| 6 | ND | ND |
| 7 | 10.0 | 31.2 |
| 8 | NC | NC |
| 9 | NC | NC |
| 10 | 0.46 | 0.83 |
| 11 | ND | ND |
| 12 | ND | ND |
| 13 | 109 | NC |
| 14 | 2.44 | 1.25 |
| 15 | 3.43 | 1.53 |
| 16 | ND | ND |
| 17 | NC | NC |
| 18 | 1.53 | 0.84 |
| 19 | 27.4 | 21.4 |
| 20 | 3.65 | 1.69 |
| 21 | 2.23 | 3.78 |
| 22 | 2.83 | 3.78 |
| 23 | 3.19 | NC |
| 24 | ND | ND |

NC = not calculated (poor fit);
ND = not determined

### In-Vivo Assays

### Biological Example V1

### Hershberger Assay

The effect of AR antagonists on androgen dependent signaling in vivo is assessed using the Hershberger assay. In this assay, peripubertal castrated male Sprague-Dawley rats are administered AR antagonists described herein in the presence of testosterone (0.4 mg/kg testosterone propionate) and the weights of androgen dependent organs measured. Dosing is continued for 10 days and measurements taken 24 h after the last dose. The extent of antagonism of AR and consequent inhibition of organ growth is evaluated by comparison to the castration control. Compounds of Formula (I) are dosed orally QD and an endpoint assessment made by change in weight of 5 androgen sensitive organs (ASO): Paired Cowper's Glands (CG), Seminal Vesicles with Fluids and Coagulating Glands (SVCG), Glans Penis (GP), Ventral Prostate (VP) and Levator Ani-Bulbocavernosus Complex (LABC)). According to assay guidelines, statistically significant suppression of ASO is required in 2 of 5 organs for a compound to be classified as an anti-androgen (analysis was performed by t-test/Mann-Whitney).

Unless otherwise stated, compounds defined herein are administered at 30 mg/kg and flutamide (FT), positive control, at 3 mg/kg. All compounds are co-administered with testosterone propionate (TP, 0.4 mg/kg) which is also administered alone, untreated control, (castrated only rats serve as the control for complete androgen blockade). A statistically significant change in ASO achieved in at least 2 of 5 organs is indicative of an active compound. Data for the inhibition of growth of the Seminal Vesicle and Coagulating Glands (SVCG) and Ventral Prostate (VP) is reported for all studies (mean organ weight (% of TP control)±SD (n=6)).

### Biological Example V2

### Castrate Resistant Prostate Cancer Xenograft Studies

Castrated six to seven week old male SCID Hairless Outbred mice (SHO, Charles Rivers Laboratories) are used as the host strain for xenograft studies. LNCaP SRαF876L cells are cultured as 3-D spheroids and expanded prior to subcutaneous injection on the flank of the animals (supplied post castration). Briefly, 5 mls of cells in media+5 mL of cultrex are premixed prior to plating of 500 μL=2×10⁵ cells per well of a 24-well plate. Plates are incubated @ 37° C. for 30 min before addition of complete media on top and incubation for growth of 3-D colonies. After 7 days, media is removed, plates chilled and contents of each well, 500 μL cultrex and cells, injected into flank of a recipient mouse. Tumor volume (length×width 2/2) is monitored weekly. When tumors reach an average volume of −200 mm³, animals are randomized into treatment groups. During the treatment period tumor volume is monitored bi-weekly. At study end, tumor growth inhibition (TGI) is calculated: 100−(Treated/Control*100). At the termination of study tumors are collected and stored for further analyses.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

We claim:

1. A compound of Formula (I) 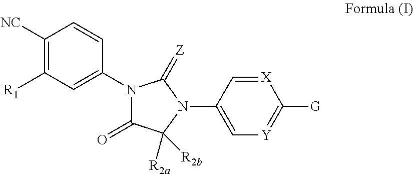
wherein
Z is S or O;
R₁ is selected from the group consisting of chloro, methyl, methoxy, difluoromethyl, and trifluoromethyl;
R₂ₐ and R_{2b} are independently C₁₋₆alkyl; or, R₂ₐ and R_{2b} are taken together with the carbon atom to which they are attached to form an unsubstituted or substituted C₃-C₁₀cycloalkyl or an unsubstituted or substituted C₃-C₁₀heterocyclyl selected from the group consisting of pyrrolidinyl and piperidinyl; wherein said substituted C₁₀cycloalkyl or substituted C₃-C₁₀heterocyclyl are optionally independently substituted with a C₁₋₃alkyl or cyclopropyl substituent;
X is C or N;
Y is C or N;
G is selected from the group consisting of g1 and g2 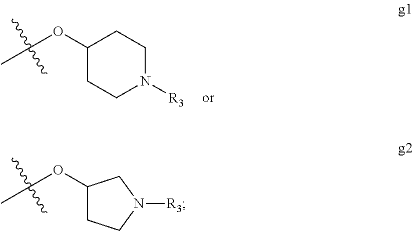
wherein R₃ is selected from the group consisting of hydrogen; C₁₋₆alkyl optionally independently substituted with a substituent selected from hydroxy, methoxy, cyano, or fluoro; C₃₋₆cycloalkyl optionally independently substituted with a substituent selected from hydroxy or fluoro; and —C(O)OR₄, wherein R₄ is C₁₋₆alkyl or —CH₂(C₆₋₁₀aryl) wherein C₆₋₁₀aryl is optionally substituted with a methoxy substituent;
such that a substituent on C₁₋₆alkyl or C₃₋₆cycloalkyl is attached at a carbon atom other than the carbon atom directly attached to the G-nitrogen atom;
wherein any nitrogen-containing heterocyclic substituent of G is optionally substituted with an oxido substituent to form an N-oxide; or an enantiomer, diastereomer, or pharmaceutically acceptable salt form thereof.

2. The compound of claim 1 wherein Z is S.

3. The compound of claim 1 wherein R₁ is selected from the group consisting of chloro, methyl, methoxy, and trifluoromethyl.

4. The compound of claim 3 wherein R₁ is chloro, methyl, or trifluoromethyl.

5. The compound of claim 4 wherein R₁ is chloro or trifluoromethyl.

6. The compound of claim 1 wherein R₂ₐ and R_{2b} are independently methyl; or, R₂ₐ and R_{2b} are taken together with the carbon atom to which they are attached to form an unsubstituted cyclobutyl ring.

7. The compound of claim 1 wherein X is C.

8. The compound of claim 7 wherein Y is N.

9. The compound of claim 1 wherein G is selected from the group consisting of g1 and g2 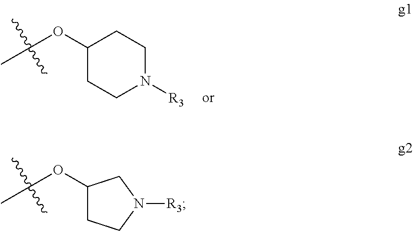
wherein R₃ is selected from the group consisting of hydrogen; C₁₋₃alkyl optionally independently substituted with a substituent selected from hydroxy, methoxy, or fluoro; C₃₋₆cycloalkyl optionally independently substituted with a substituent selected from hydroxy or fluoro; and —C(O)OR₄, wherein R₄ is C₁₋₆alkyl or —CH₂(phenyl), and wherein the phenyl is optionally substituted with a methoxy substituent;
such that a substituent on C₁₋₆alkyl or C₃₋₆cycloalkyl is attached at a carbon atom other than the carbon atom directly attached to the G-nitrogen atom.

10. The compound of claim 9 wherein G is selected from the group consisting of g1 and g2 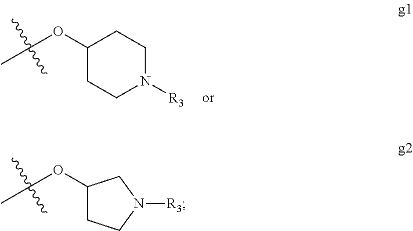
wherein R₃ is selected from the group consisting of hydrogen; C₁₋₃alkyl optionally independently substituted with a substituent selected from methoxy or fluoro; and —C(O)OR₄, wherein R₄ is C₁₋₆alkyl or —CH₂(phenyl) and wherein the phenyl is optionally substituted with a methoxy substituent;
such that a substituent on C₁₋₃alkyl is attached at a carbon atom other than the carbon atom directly attached to the G-nitrogen atom.

11. The compound of claim 10 wherein G is selected from the group consisting of g1 and g2 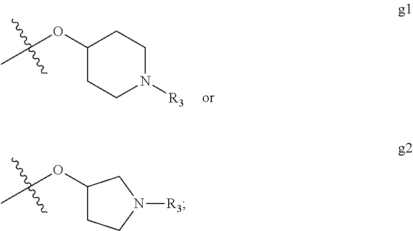
wherein R₃ is selected from the group consisting of hydrogen; methyl, and —C(O)OR₄, wherein R₄ is C₁₋₄alkyl or —CH₂(phenyl).

12. The compound of claim 11 wherein G is g1 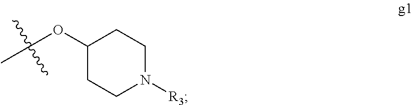
wherein R₃ is selected from the group consisting of hydrogen;
methyl, and —C(O)OR₄, and wherein R₄ is C₁₋₄alkyl or —CH₂(phenyl).

13. The compound of claim 12 wherein G is g1 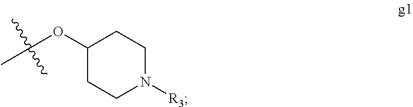
wherein R₃ is selected from the group consisting of hydrogen and methyl.

14. A compound of Formula (I) 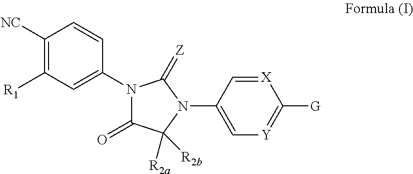
wherein
Z is S;
R₁ is selected from the group consisting of chloro, methyl, methoxy, and trifluoromethyl;
R₂ₐ and R_{2b} are independently methyl; or, R₂ₐ and R_{2b} are taken together with the carbon atom to which they are attached to form an unsubstituted cyclobutyl ring;
X is C or N;
Y is C or N;
G is selected from the group consisting of g1 and g2 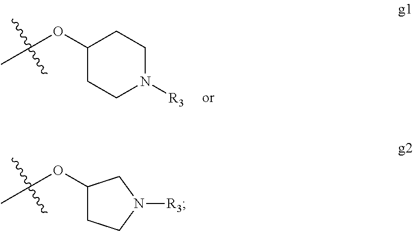
wherein R₃ is selected from the group consisting of hydrogen; C₁₋₄alkyl optionally independently substituted with a substituent selected from hydroxy, methoxy, or fluoro; C₃₋₆cycloalkyl optionally independently substituted with a substituent selected from hydroxy or fluoro; and —C(O)OR₄, wherein R₄ is C₁₋₆alkyl or —CH₂(phenyl), and wherein the phenyl is optionally substituted with a methoxy substituent;
such that a substituent on C₁₋₄alkyl or C₃₋₆cycloalkyl is attached at a carbon atom other than the carbon atom directly attached to the G-nitrogen atom;
wherein any nitrogen-containing heterocyclic substituent of G is optionally substituted with an oxido substituent to form an N-oxide; or an enantiomer, diastereomer, or pharmaceutically acceptable salt form thereof.

15. A compound of Formula (I) 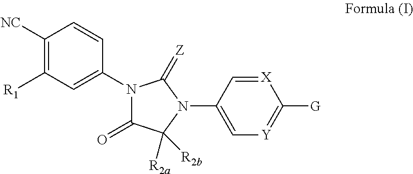
wherein
Z is S;
R₁ is chloro, methyl, or trifluoromethyl;
R₂ₐ and R_{2b} are independently methyl; or, R₂ₐ and R_{2b} are taken together with the carbon atom to which they are attached to form an unsubstituted cyclobutyl ring;
X is C or N;
Y is C or N;
G is selected from the group consisting of g1 and g2 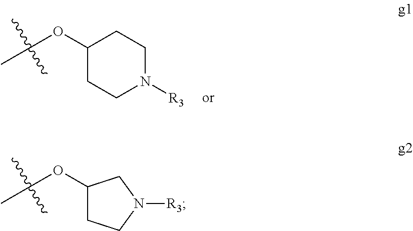
wherein R₃ is selected from the group consisting of hydrogen; C₁₋₃alkyl optionally independently substituted with a substituent selected from methoxy or fluoro; and —C(O)OR₄, wherein R₄ is C₁₋₆alkyl or —CH₂(phenyl) and wherein the phenyl is optionally substituted with a methoxy substituent;
such that a substituent on C₁₋₃alkyl is attached at a carbon atom other than the carbon atom directly attached to the G-nitrogen atom;
wherein any nitrogen-containing heterocyclic substituent of G is optionally substituted with an oxido substituent to form an N-oxide; or an enantiomer, diastereomer, or pharmaceutically acceptable salt form thereof.

16. A compound of Formula (I) 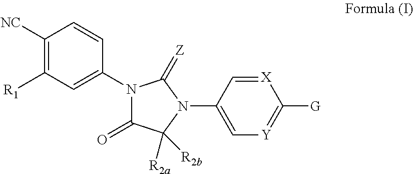
wherein
Z is S;
R₁ is chloro or trifluoromethyl;
R₂ₐ and R_{2b} are independently methyl; or, R₂ₐ and R_{2b} are taken together with the carbon atom to which they are attached to form an unsubstituted cyclobutyl ring;
X is C or N;
Y is C or N;
G is selected from the group consisting of g1 and g2 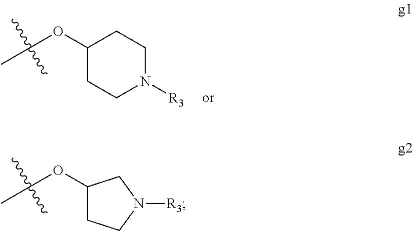
wherein R₃ is selected from the group consisting of hydrogen; methyl, and —C(O)OR₄, wherein R₄ is C₁₋₄alkyl or —CH₂(phenyl);
wherein any nitrogen-containing heterocyclic substituent of G is optionally substituted with an oxido substituent to form an N-oxide; or an enantiomer, diastereomer, or pharmaceutically acceptable salt form thereof.

17. A compound of Formula (I) 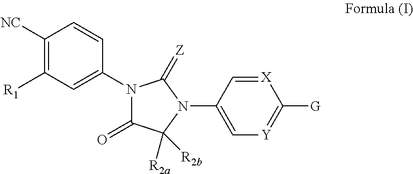
wherein
Z is S;
R₁ is chloro or trifluoromethyl;
R₂ₐ and R_{2b} are independently methyl; or, R₂ₐ and R_{2b} are taken together with the carbon atom to which they are attached to form an unsubstituted cyclobutyl ring;
X is C;
Y is N;
G is selected from the group consisting of g1 and g2 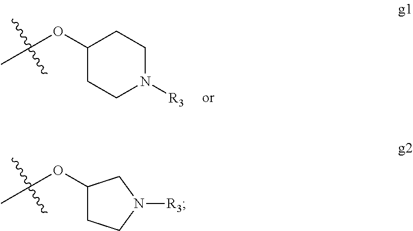
wherein R₃ is selected from the group consisting of hydrogen; methyl, and —C(O)OR₄, wherein R₄ is C₁₋₄alkyl or —CH₂(phenyl);
wherein any nitrogen-containing heterocyclic substituent of G is optionally substituted with an oxido substituent to form an N-oxide; or an enantiomer, diastereomer, or pharmaceutically acceptable salt form thereof.

18. A compound of Formula (I) 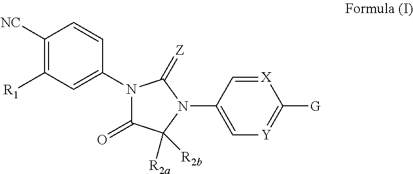
wherein
Z is S;
R₁ is chloro or trifluoromethyl;
R₂ₐ and R_{2b} are independently methyl; or, R₂ₐ and R_{2b} are taken together with the carbon atom to which they are attached to form an unsubstituted cyclobutyl ring;
X is C;
Y is N;
G is g1 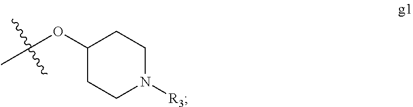
wherein R₃ is selected from the group consisting of hydrogen; methyl, and —C(O)OR₄, wherein R₄ is C₁₋₄alkyl or —CH₂(phenyl);
wherein any nitrogen-containing heterocyclic substituent of G is optionally substituted with an oxido substituent to form an N-oxide; or an enantiomer, diastereomer, or pharmaceutically acceptable salt form thereof.

19. A compound of Formula (I) 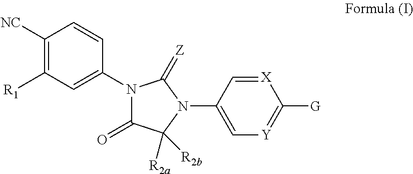
wherein
Z is S;
R₁ is chloro or trifluoromethyl;
R₂ₐ and R_{2b} are independently methyl; or, R₂ₐ and R_{2b} are taken together with the carbon atom to which they are attached to form an unsubstituted cyclobutyl ring;
X is C;
Y is N;
G is g1 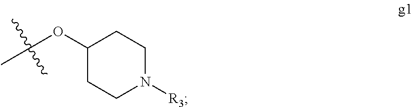
wherein R₃ is selected from the group consisting of hydrogen and methyl;
wherein any nitrogen-containing heterocyclic substituent of G is optionally substituted with an oxido substituent to form an N-oxide; or an enantiomer, diastereomer, or pharmaceutically acceptable salt form thereof.

20. A compound of Formula (I) 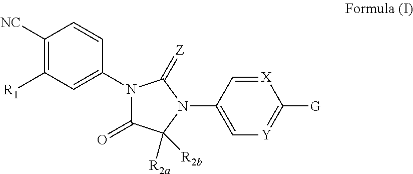
selected from the group consisting of
5-[8-[6-[(1-methyl-4-piperidyl)oxy]-3-pyridyl]-5-oxo-7-thioxo-6,8-diazaspiro[3.4]octan-6-yl]-3-(trifluoromethyl)pyridine-2-carbonitrile;
3-methyl-5-[8-[6-[(1-methyl-4-piperidyl)oxy]-3-pyridyl]-5-oxo-7-thioxo-6,8-diazaspiro[3.4]octan-6-yl]pyridine-2-carbonitrile;
4-(4,4-Dimethyl-5-oxo-3-(6-(piperidin-4-yloxy)pyridin-3-yl)-2-thioxoimidazolidin-1-yl)-2-(trifluoromethyl)benzonitrile;
4-(4,4-Dimethyl-3-(6-((1-methylpiperidin-4-yl)oxy)pyridin-3-yl)-5-oxo-2-thioxoimidazolidin-1-yl)-2-(trifluoromethyl)benzonitrile;
4-[4,4-Dimethyl-5-oxo-3-[2-(4-piperidinyloxy)pyrimidin-5-yl]-2-thioxo-imidazolidin-1-yl]-2-(trifluoromethyl)benzonitrile;
4-(4,4-Dimethyl-3-(2-((1-methylpiperidin-4-yl)oxy)pyrimidin-5-yl)-5-oxo-2-thioxoimidazolidin-1-yl)-2-(trifluoromethyl)benzonitrile;
2-Chloro-4-(4,4-dimethyl-5-oxo-3-(4-(piperidin-4-yloxy)phenyl)-2-thioxoimidazolidin-1-yl)benzonitrile;
2-Chloro-4-(4,4-dimethyl-3-(4-((1-methylpiperidin-4-yl)oxy)phenyl)-5-oxo-2-thioxoimidazolidin-1-yl)benzonitrile;
2-Chloro-4-(4,4-dimethyl-5-oxo-3-(6-(piperidin-4-yloxy)pyridin-3-yl)-2-thioxoimidazolidin-1-yl)benzonitrile hydrochloride;
2-Chloro-4-(4,4-dimethyl-3-(6-((1-methylpiperidin-4-yl)oxy)pyridin-3-yl)-5-oxo-2-thioxoimidazolidin-1-yl)benzonitrile;
4-(8-Oxo-5-(4-(piperidin-4-yloxy)phenyl)-6-thioxo-5,7-diazaspiro[3.4]octan-7-yl)-2-(trifluoromethyl)benzonitrile;
4-(5-(4-((1-Methylpiperidin-4-yl)oxy)phenyl)-8-oxo-6-thioxo-5,7-diazaspiro[3.4]octan-7-yl)-2-(trifluoromethyl)benzonitrile;
4-(8-Oxo-5-(6-(piperidin-4-yloxy)pyridin-3-yl)-6-thioxo-5,7-diazaspiro[3.4]octan-7-yl)-2-(trifluoromethyl)benzonitrile;
4-(5-(6-((1-Methylpiperidin-4-yl)oxy)pyridin-3-yl)-8-oxo-6-thioxo-5,7-diazaspiro[3.4]octan-7-yl)-2-(trifluoromethyl)benzonitrile;
4-(8-Oxo-5-(2-(piperidin-4-yloxy)pyrimidin-5-yl)-6-thioxo-5,7-diazaspiro[3.4]octan-7-yl)-2-(trifluoromethyl)benzonitrile;
4-(5-(2-((1-Methylpiperidin-4-yl)oxy)pyrimidin-5-yl)-8-oxo-6-thioxo-5,7-diazaspiro[3.4]octan-7-yl)-2-(trifluoromethyl)benzonitrile;
2-Methyl-4-(5-(4-((1-methylpiperidin-4-yl)oxy)phenyl)-8-oxo-6-thioxo-5,7-diazaspiro[3.4]octan-7-yl)benzonitrile;
2-Methyl-4-(5-(6-((1-methylpiperidin-4-yl)oxy)pyridin-3-yl)-8-oxo-6-thioxo-5,7-diazaspiro[3.4]octan-7-yl)benzonitrile;
2-Methoxy-4-(5-(4-((1-methylpiperidin-4-yl)oxy)phenyl)-8-oxo-6-thioxo-5,7-diazaspiro[3.4]octan-7-yl)benzonitrile;
2-Methoxy-4-(5-(6-((1-methylpiperidin-4-yl)oxy)pyridin-3-yl)-8-oxo-6-thioxo-5,7-diazaspiro[3.4]octan-7-yl)benzonitrile;
2-Chloro-4-(8-oxo-5-(4-(piperidin-4-yloxy)phenyl)-6-thioxo-5,7-diazaspiro[3.4]octan-7-yl)benzonitrile;
2-Chloro-4-(5-(4-((1-methylpiperidin-4-yl)oxy)phenyl)-8-oxo-6-thioxo-5,7-diazaspiro[3.4]octan-7-yl)benzonitrile;
2-Chloro-4-(8-oxo-5-(6-(piperidin-4-yloxy)pyridin-3-yl)-6-thioxo-5,7-diazaspiro[3.4]octan-7-yl)benzonitrile;
and
2-Chloro-4-(5-(6-((1-methylpiperidin-4-yl)oxy)pyridin-3-yl)-8-oxo-6-thioxo-5,7-diazaspiro[3.4]octan-7-yl)benzonitrile;
or a pharmaceutically acceptable salt form thereof.

21. A compound of claim 18 selected from the group consisting of 4-(4,4-dimethyl-5-oxo-3-(6-(piperidin-4-yloxy)pyridin-3-yl)-2-thioxoimidazolidin-1-yl)-2-(trifluoromethyl)benzonitrile, 4-(4,4-dimethyl-3-(6-((1-methylpiperidin-4-yl)oxy)pyridin-3-yl)-5-oxo-2-thioxoimidazolidin-1-yl)-2-(trifluoromethyl)benzonitrile, 4-(8-oxo-5-(6-(piperidin-4-yloxy)pyridin-3-yl)-6-thioxo-5,7-diazaspiro[3.4]octan-7-yl)-2-(trifluoromethyl)benzonitrile, and 4-(5-(6-((1-methylpiperidin-4-yl)oxy)pyridin-3-yl)-8-oxo-6-thioxo-5,7-diazaspiro[3.4]octan-7-yl)-2-(trifluoromethyl)benzonitrile.

22. A pharmaceutical composition comprising a compound of claim 1 or 20 and at least one of a pharmaceutically acceptable carrier, a pharmaceutically acceptable excipient, and a pharmaceutically acceptable diluent.

23. The pharmaceutical composition of claim 20, wherein the composition is a solid oral dosage form.

24. The pharmaceutical composition of claim 22, wherein the composition is a syrup, an elixir or a suspension.

25. A pharmaceutical composition comprising a compound of claim 21 and at least one of a pharmaceutically acceptable carrier, a pharmaceutically acceptable excipient, and a pharmaceutically acceptable diluent.

26. A pharmaceutical composition comprising a compound of Formula (I) and abiraterone acetate.

27. The pharmaceutical composition of claim 26 further comprising prednisone.

28. A method of treating a disease, syndrome, condition, or disorder in a subject in need thereof, in which the disease, the syndrome, the condition or the disorder is affected by the antagonism of one or more androgen receptor types, by administering to the subject in need thereof a therapeutically effective amount of a compound, or pharmaceutically acceptable salt form thereof, of Formula (I).

29. The method of claim 28 wherein the disease, syndrome, condition, or disorder is selected from the group consisting of prostate cancer, castration-resistant prostate cancer, and metastatic castration-resistant prostate cancer.

30. The method of claim 29 wherein the disease, syndrome, condition, or disorder is prostate cancer.

31. The method of claim 29 wherein the disease, syndrome, condition, or disorder is castration-resistant prostate cancer.

32. The method of claim 29 wherein the disease, syndrome, condition, or disorder is metastatic castration-resistant prostate cancer.

33. A method of treating a disorder selected from the group consisting of prostate cancer, castration-resistant prostate cancer, and metastatic castration-resistant prostate cancer, comprising administering to a subject in need thereof a therapeutically effective amount of the composition of claim 1.

34. The use of a compound as in claim 1 for the preparation of a medicament for treating a disease, syndrome, condition, or disorder is selected from the group consisting of prostate cancer, castration-resistant prostate cancer, and metastatic castration-resistant prostate cancer.

35. The use of a compound as in claim 1, as a method for treating a disease, syndrome, condition, or disorder selected from the group consisting of prostate cancer, castration-resistant prostate cancer, and metastatic castration-resistant prostate cancer, in a subject in need thereof.
